SEQUENCE LISTING (6 sequences)

SEQ ID NO: 1 - DNA, 2571 bp, murine
atgagagaggcttatctcagatgttggatcttctcttggaaaaacgtgtgggtacgaccttgccaaaggctgcattttaaaaccgtgcttcttcaaggcagtctactttacacggctttggactcttactcaactgtacaagctgcccccaagtctagctccggctccgtgaagtttcaaggactggcagagactgggatcatgaaaatggacatggaggacgctgatatgactttgtggacagaggccgagtttgaagagaagtgtacatacatagtgaacgaccacccctgggattccggcgctgacgggggtacttctgttcaagccgaggcatccttaccaaggaacctgcttttcaagtatgctgccaacaacagcaaagaggttattggcgtggtaagtaaggagtacataccgaagggaacacgctttggacccctcatcggtgaagtctacactaatgacacagttcccaagaatgccaacaggaagtatttttggcggatctattccagagaggagttccaccacttcattgatggctttaatgaggagaaaagcaactggatgcgctacgtgaatccagctcactctgcccgggagcaaaacctggctgcctgtcagaacgggatgaacatctacttctacactattaagcctatccctgccaaccaggaacttcttgtgtggtattgtcgggactttgcggagaggctccactacccttatcctggagagctcacagtgataaatctcacacaaacggaaagcaacccaaagcaatacagtagtgagaaaaatgaactctacccaaagagtgtccccaagagagagtacagcgtgaaagaaattctaaaactggactccaatccctccaaaaggaaggacatctaccgttccaacatttcacccttcactttagaaaaggacatggatggctttcggaaaaatgggagccccgacatgcccttctaccctcgggtggtttatcctatccgggcacctctgccagaagactttttgaaagcgtccctggcctatgggatggagagacccacctacataactcacagtccccttccgtcttccacaactccaagtccccctgcgagcagcagcccggagcagagccttaagagctccagcccccacagcagcccgggaaacacggtgtcacccctggcgccaggcctcccagaacaccgggactcctactcctacttgaatgtttcctatggttccgagggcctgggctcctaccctggctatgcacctgccccccacctcccaccagctttcattccttcttacaatgctcactaccccaagttcctgttgccaccgtacggcattagttccaatggcttgagcaccatgaacaacatcaatggtatcaacaacttcagcctcttccctaggttgtatcccgtctacagtaacctccttagtggcagcagcctgcctcatcccatgctcaatccagcttccctaccgagttccctgcctaccgatggagcccggaggctgcttccaccggagcaccccaaagaggtgcttatcccagcaccccacagtgccttctcccttaccggggctgcagccagcatgaaggacgagagtagtccccccagcggatctccaacggcgggaactgcagccacgtcagaacacgtggtacaacccaaagctacctcatcagtgatggcggcccccagcactgacggagccatgaatctcattaaaaacaaacgaaacatgactggttacaagactcttccttaccctctgaagaaacagaatggcaagatcaagtatgagtgcaatgtctgtgccaagacgttcggtcagctctccaacctgaaggtccacctgagagtgcacagtggagaacggcctttcaagtgccagacctgcaacaagggttttactcagctcgcccacctgcagaaacactacttggtacacacaggagagaagccacatgagtgccaggtctgccacaagagatttagcagcacaagcaatctcaagacccaccttcgattgcattctggagaaaaaccttaccaatgtaaggtgtgccctgccaagtttacgcaatttgtgcacctgaagctgcacaagcgactgcatacccgggagcggcctcacaagtgtgcccagtgtcacaagagctacatccatctctgcagcctcaaggtccacctgaagggcaactgccctgcgggcccagctgctgggctgcctttggaggatctgacccgaatcaatgaagaaattgagaggttcgacatcagcgacaatgcagaccgtcttgaggacatggaggacagtgtcgatgtgacctccatggtggagaaggagattctagctgtggtcagaaaagagaaagaagaaaccagtctgaaagtgtctttgcaaagaaacatggggaacggcctcctctcctcagggtgcagcctctatgagtcatcggacctgtccctcatgaagttgcctcacagcaacccactacctctggtgcctgtaaaggtcaaacaagaaacagttgaaccgatggatccttaa

SEQ ID NO: 2 - protein, 856 aa, murine
MREAYLRCWIFSWKNVWVRPCQRLHFKTVLLQGSLLYTALDSYSTVQAAPKSSSGSVKFQGLAETGIMKMDMEDADMTLWTEAEFEEKCTYIVNDHPWDSGADGGTSVQAEASLPRNLLFKYAANNSKEVIGVVSKEYIPKGTRFGPLIGEVYTNDTVPKNANRKYFWRIYSREEFHHFIDGFNEEKSNWMRYVNPAHSAREQNLAACQNGMNIYFYTIKPIPANQELLVWYCRDFAERLHYPYPGELTVINLTQTESNPKQYSSEKNELYPKSVPKREYSVKEILKLDSNPSKRKDIYRSNISPFTLEKDMDGFRKNGSPDMPFYPRVVYPIRAPLPEDFLKASLAYGMERPTYITHSPLPSSTTPSPPASSSPEQSLKSSSPHSSPGNTVSPLAPGLPEHRDSYSYLNVSYGSEGLGSYPGYAPAPHLPPAFIPSYNAHYPKFLLPPYGISSNGLSTMNNINGINNFSLFPRLYPVYSNLLSGSSLPHPMLNPASLPSSLPTDGARRLLPPEHPKEVLIPAPHSAFSLTGAAASMKDESSPPSGSPTAGTAATSEHVVQPKATSSVMAAPSTDGAMNLIKNKRNMTGYKTLPYPLKKQNGKIKYECNVCAKTFGQLSNLKVHLRVHSGERPFKCQTCNKGFTQLAHLQKHYLVHTGEKPHECQVCHKRFSSTSNLKTHLRLHSGEKPYQCKVCPAKFTQFVHLKLHKRLHTRERPHKCAQCHKSYIHLCSLKVHLKGNCPAGPAAGLPLEDLTRINEEIERFDISDNADRLEDMEDSVDVTSMVEKEILAVVRKEKEETSLKVSLQRNMGNGLLSSGCSLYESSDLSLMKLPHSNPLPLVPVKVKQETVEPMDP

SEQ ID NO: 3 - DNA, 2370 bp, human
atgaaaatggacatggaggatgcggatatgactctgtggacagaggctgagtttgaagagaagtgtacatacattgtgaacgaccacccctgggattctggtgctgatggcggtacttcggttcaggcggaggcatccttaccaaggaatctgcttttcaagtatgccaccaacagtgaagaggttattggagtgatgagtaaagaatacataccaaagggcacacgttttggacccctaataggtgaaatctacaccaatgacacagttcctaagaacgccaacaggaaatatttttggaggatctattccagaggggagcttcaccacttcattgacggctttaatgaagagaaaagcaactggatgcgctatgtgaatccagcacactctccccgggagcaaaacctggctgcgtgtcagaacgggatgaacatctacttctacaccattaagcccatccctgccaaccaggaacttcttgtgtggtattgtcgggactttgcagaaaggcttcactacccttatcccggagagctgacaatgatgaatctcacacaaacacagagcagtctaaagcaaccgagcactgagaaaaatgaactctgcccaaagaatgtcccaaagagagagtacagcgtgaaagaaatcctaaaattggactccaacccctccaaaggaaaggacctctaccgttctaacatttcacccctcacatcagaaaaggacctcgatgactttagaagacgtgggagccccgaaatgcccttctaccctcgggtcgtttaccccatccgggcccctctgccagaagactttttgaaagcttccctggcctacgggatcgagagacccacgtacatcactcgctcccccattccatcctccaccactccaagcccctctgcaagaagcagccccgaccaaagcctcaagagctccagccctcacagcagccctgggaatacggtgtcccctgtgggccccggctctcaagagcaccgggactcctacgcttacttgaacgcgtcctacggcacggaaggtttgggctcctaccctggctacgcacccctgccccacctcccgccagctttcatcccctcgtacaacgctcactaccccaagttcctcttgcccccctacggcatgaattgtaatggcctgagcgctgtgagcagcatgaatggcatcaacaactttggcctcttcccgaggctgtgccctgtctacagcaatctcctcggtgggggcagcctgccccaccccatgctcaaccccacttctctcccgagctcgctgccctcagatggagcccggaggttgctccagccggagcatcccagggaggtgcttgtcccggcgccccacagtgccttctcctttaccggggccgccgccagcatgaaggacaaggcctgtagccccacaagcgggtctcccacggcgggaacagccgccacggcagaacatgtggtgcagcccaaagctacctcagcagcgatggcagcccccagcagcgacgaagccatgaatctcattaaaaacaaaagaaacatgaccggctacaagacccttccctacccgctgaagaagcagaacggcaagatcaagtacgaatgcaacgtttgcgccaagactttcggccagctctccaatctgaaggtccacctgagagtgcacagtggagaacggcctttcaaatgtcagacttgcaacaagggctttactcagctcgcccacctgcagaaacactacctggtacacacgggagaaaagccacatgaatgccaggtctgccacaagagatttagcagcaccagcaatctcaagacccacctgcgactccattctggagagaaaccataccaatgcaaggtgtgccctgccaagttcacccagtttgtgcacctgaaactgcacaagcgtctgcacacccgggagcggccccacaagtgctcccagtgccacaagaactacatccatctctgtagcctcaaggttcacctgaaagggaactgcgctgcggccccggcgcctgggctgcccttggaagatctgacccgaatcaatgaagaaatcgagaagtttgacatcagtgacaatgctgaccggctcgaggacgtggaggatgacatcagtgtgatctctgtagtggagaaggaaattctggccgtggtcagaaaagagaaagaagaaactggcctgaaagtgtctttgcaaagaaacatggggaatggactcctctcctcagggtgcagcctttatgagtcatcagatctacccctcatgaagttgcctcccagcaacccactacctctggtacctgtaaaggtcaaacaagaaacagttgaaccaatggatccttaa

SEQ ID NO: 4 - protein, 789 aa, human
MKMDMEDADMTLWTEAEFEEKCTYIVNDHPWDSGADGGTSVQAEASLPRNLLFKYATNSEEVIGVMSKEYIPKGTRFGPLIGEIYTNDTVPKNANRKYFWRIYSRGELHHFIDGFNEEKSNWMRYVNPAHSPREQNLAACQNGMNIYFYTIKPIPANQELLVWYCRDFAERLHYPYPGELTMMNLTQTQSSLKQPSTEKNELCPKNVPKREYSVKEILKLDSNPSKGKDLYRSNISPLTSEKDLDDFRRRGSPEMPFYPRVVYPIRAPLPEDFLKASLAYGIERPTYITRSPIPSSTTPSPSARSSPDQSLKSSSPHSSPGNTVSPVGPGSQEHRDSYAYLNASYGTEGLGSYPGYAPLPHLPPAFIPSYNAHYPKFLLPPYGMNCNGLSAVSSMNGINNFGLFPRLCPVYSNLLGGGSLPHPMLNPTSLPSSLPSDGARRLLQPEHPREVLVPAPHSAFSFTGAAASMKDKACSPTSGSPTAGTAATAEHVVQPKATSAAMAAPSSDEAMNLIKNKRNMTGYKTLPYPLKKQNGKIKYECNVCAKTFGQLSNLKVHLRVHSGERPFKCQTCNKGFTQLAHLQKHYLVHTGEKPHECQVCHKRFSSTSNLKTHLRLHSGEKPYQCKVCPAKFTQFVHLKLHKRLHTRERPHKCSQCHKNYIHLCSLKVHLKGNCAAAPAPGLPLEDLTRINEEIEKFDISDNADRLEDVEDDISVISVVEKEILAVVRKEKEETGLKVSLQRNMGNGLLSSGCSLYESSDLPLMKLPPSNPLPLVPVKVKQETVEPMDP

SEQ ID NO: 5 - DNA, 20894 bp, murine
  misc_feature (2935)..(3024): n is any nucleotide
gggggaagagtagtcagtcgctcgctcactcgctcgctcgcacagacactgctgcagtgacactcggccctccagtgtcgcggagacgcaagagcagcgcgcagcacctgtccgcccggagcgagcccggcccgcggccgtagaaaaggagggaccgccgaggtgcgcgtcagtactgctcagcccggcagggacgcgggaggatgtggactgggtggacatgagagaggcttatctcagatgttggatcttctcttggaaaaacgtgtgggtacgaccttggtaaggaaccagattctgtctttaatacgatttgaaaccttttatccctttttctttcttccttttttttttaacttttcttttctcccccctcccccttttaaaaaaaaaaaaagaatgaagcctcagtagaaaccagcgcttctgttttagtacgcggagcactgtcaaacatttagaagacttttttcctccgtatgaatcattaaccctttcagttctagacataattgtcaattcactgaaatttcagtagtggttcttgtccgcttcgccactcgctgcctttacattactgtaactatcccgggttgacttaggttttcacttgtatttaacatcgtttgttccacatggaccttacatgttggaactaaataagaatgagatagtttaagttgtacccgggacaaggacaagtaagcatctttccccttctcggagcgtcctatctagggacgaattgtaaagaccagctccggagagggactcccgctgtactgtgtttacattttcacaagcgcgcgttctaacatggttatccttattcctaatttttatctgcggcgtctatgtgggaatacgttgcagaggctgttttatctttcttgcttttcctctttggaaaggactttttccgagggcagataagaggaggatccccaagtcttctgtataactttagttacagtaaactgtgccacttcagtgacttctgggaattcatgcactttcacatttaaatagaaagtgctatttgtggctgagggctcctaaaggaattctcttcagggaattctattgactttttttttaatatgttttgtttttaatttttctatctggctcgagatgcccacggattaaaaaaacaaacaaaactgctgggtgtttccctcttccccaatttttcttttcctgtggtccatgggagctcgggaaggctggtactcaaggatgctggcaggatgcaacccctctcaggcttgcctgctggaggatgaaagagactgaacgcgcgcgcgcgcgcggcagagggaggggacctagttgttttgaaagttgcttcgctagggagctggtgggaaagttcagttttccccatttggaaaaggcagactgggttccgctcctgcaccacacgtgttttccatttttagcttcattcagacgcaggcagcgcccctgcctcttcctccccttgtttgtgacacttctctgagacagcttttccacagctctgagggtctggcggccatgaccccgggcgtcccgggacacaggacgcagcagcgcccacaacacatttctgccttgagtgataaagccaaggattgttcaaaggtagctgttctttctctcccgatgaggttaacatatacatatacgctttttttttttttttcagccaaaggctgcattttaaaaccgtgcttcttcaaggcagtctactttacacggctttggactcttactcaactgtacaagtactccaagcttttaaagtcttcagagcaccgtgttagtcatagcctctaagagggaggcacaggagcgccggacaatggggattaaaagcctttcccttctcttccaggctgcccccaagtctagctccggctccgtgaagtttcaaggactggcagagactgggatcatgaaaatggacatggaggacgctgatatgactttgtggacagaggccgagtttgaagagaagtgtacatacatagtgaacgaccacccctgggattccggcgctgacgggggtacttctgttcaagccgaggcatccttaccaaggaacctgcttttcaagtatgctgccaacaacagcaaagaggtaagccggctgccttcttgaagtctgactggcaattgggccagctctcctactactatctctgagaaccgtgagaatttatatgcattggcaaataattgatcgctccagtggctgttttccttgctttctcttcaaaccaattcctattcatttcttcctcccttcagctgtcctatactaattagtaaacagttaaattttttggcaagttgacatgtcttgggaaagctaactggcagcactggtgggcagcatggtaaagggctcagtgcttcaccccctggccctcttggatgacagttttaaaggaaagaaacttccttagaaaaagaagtttttcctctgctcatgagatggctttattcttttaacgagccagctttattagctgggtttctaaaattattctcaaaaccttgacgtgtttatgaactgaagagatggcattaaccaggaagagggtcacgtaaaagtgtcctctgtcaggatgacttcactaaccaccctttacctgtggcagctccctggcctgggccaggccggcaggtccatgttttatggcttctgaagtgggtacactctttgtatcaaagacacagaacacctgaggagcacctgatttgtgtttatataacaattagagtcggctgtgaagtgatttgcaaaataactccttgctctgagaatctggctgctgcagttgctctcctgatggcttaagttgctgaggctagccctgaggagacttcccaccatcaccattgcccacagtgctgtggtttctgatccttgctgtctttggggagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaggagggattctgtgttgtaaggattccagatttgctcctaggactatgcattatgaaagtgccaggtcactttcctccttccctaagcgacaacatggngaaagaaactccagagttggggaaacactgtctctttcctgtttgtgtgtctcagttcagggtttggggggggaaggagggaggtggcaaagaccgagagctaaacttaaagagaggaattctaccttgtgtttcactaaagctccactctttgttctatttctgatgggagacttatttgttttatgaaaagcaaattttaaattgtagtttgtgtataaataccaccaaaaaaaattgtaaccccagaggtcacccttaattatacctctttctgaaaacaatgttcttctcaacagaaagcctctgagtatgtaacccgtggagtcaatgtggcgtttgtttgggtatttgaggtctggctgtggctttaaaaggtgtagtgtgttctccccccatcagatttgcctaaatattggtcccttcaaaaagggccattcatgtcctgttgtctctgtagcctgttgggatagaaataaagactcctggttttttgtcatcggctctgttctccttcagagtccttctcagctcagccctataaagtttttcaatttttgttgttctactgaaaactatggctgaacggagttatgctattagatcatttagttcaaggttcagatagtcaacttccccgaaggctagagttacagccaggcccagactgggagcaaagttctctgattgctccaggtgtttctagatcatgctactctggtccctcttggtgtcactgtggctttatttatcttagcataattcaggcacagcttttcactaagcctgtttgattctgtttcttttctgctgaattccttcccatctctgtatgttaagcaaccagaaagaggcagctcttgtgtttctgacagggtgcccttcacaacacacagaaagtcagggttctttgagcctactctatgttttaccgaaagaaaagtcaaatcgttgtggtagcacctcttcggtcctcaatcatgaaagccaccaggaaacaaaatttaagcatcatcctatcctaagcatacaccaaaatgtgggcctgtttttcccttctctccagcgccttctgttttgaactttgggacaggtcccttggggtggtgaaagagaaggacagaattgagccattgagggtgtgatagctgcaaaccagttccaggccagaagcatctaggatggaggaagtgtttacattaccattgttcagttagttcggtgagttccatgagctgtagaaacagggaaaggcctttctacgggtagcttttatctgactgatctggcttgcccatctttaaatgttttcattaaaatagcaaaagctgttactttatgtactgtgtgctgacatcctggggactttttttttaaaaaaaaaaaaaacaagatcatttttcattatgaatttgtgctatgtgtcagcaccttcccacccccacccccacccccttgatttaaaaacaaaacatgcaaactgtatttctgatcttaacatcaatttttagctctggctcagaatcattctgttcttttattggggccgtttgatcattcttacttcctatttccagatctaaaaccaagtacaagcacactcttcaaaacttttagaacgtttaaccgggtgggttcttcttccgtgggatttccccctcgtcctcctgaaataatctctgaatatttgactgcattaaaaaaaaatcagttttgacattgggagagcagtatccaggacaactttctgctgtcagctgaggtggcttgtcctcaggcactgtgtggctcctctgactctggtctgaagttgagtggtgacaaacatcaggcagatctagggacatggtgtccattctgattgaagtccattattaaattgagccatctgtatggaatctcagcacaacatgcaattccacctccggacttttttatttggtagtatgtgccagagtgccaccacatctcccttcacataaagataacacgaagcaggagacctttcaaaaacacctgctaattcctttgaaaactggactgaagtacagacttgatatctcggtctatacccttatagtaaaaatacaggaaaggaaatctaattgttgggcgagatttggggaggggaaaaacagcaagtaattaaagtaaccttaatttgaagtgaggaaactcaggccacttttggaactgaatagtgtctcctttcaaatccccctgctttctactgggctcctatatcgaacactctctaaatggctgtttgttttgaaagcaaacaaacagacctatgctgcccttatcatccgactttcattctgtactcccactcccactcactccatcctgcactgtaggaatctttcttttctaaagttaaaaagaagctctgctttttgtctctgagctccagtttgtgtcctatagccaaagccactgacagagacagatgtgcccaggtggcagaggggtcggctccgcaagtcaagcaagccatgaagagcttccgtggggctgaagcatgggcagcctaaggaggcctctttctcttcctgatgggtgtagggaaggcacacagcagccagtccccaggagtaattctttaggaccctggtcagttccagtgaagtttctttcctcagttaggtgagcacactcttggtctttttagcagtgtgcaactcaaagcacaggtcaagtttctctgtgccctctgcttgttcctagggtagggcactgtaaacaatgggttaggaagatgggttgggctaagacactcatctggatcagtttcctgctttccattaagaacaaccctggctcttacccccatcctaagacaccccacaatcaccataaccttagagccgcagtttctatgtcttctttgattaaggaatcagacatggattagacgtgaaggtgtttattgtgtgttgttagcatgctgaacttgagatggctataggtggtttatttgatgttcttttttctgagatagaatcttgagatgtctgtagtcggaatgcaggccacaagctgctgatcttcctgcatgctatgattagaggcatgcaccacaccacacctagtgagacaggtgatttcgaagctacagggttcctctaacctcagaatgctgaattgtgtgtctctgtttacctatcagtgtgaatttcctcgtgttcttcagctctgtgtatagtaggaattttaaatatcaaggtcctattgtactcataaaaacaatctaatattgcattttggtctggttatatttcccagactgaccttggactcaataatctcttgcctctgcctcagagtggctgggacttgaagccaccccagaatttctggttactcctttttctctcattcacatgacttgtctgttgagttgcatttgtaaggttaaagatggcaggcgctccaaacactgacttaaacagctacagtgccaactccatagttgtgcctaaaaggtcctagatagcttgtaccttctttctttttctccccccccccccccccaagattctaaataacttccttttggccacaggacaatgtgttttgttagcgttaaaatcggtaggtgaaaaccaagcgtctcttctgtagagagaatggcaatctggaagggaagctgtgacctcattgtactgctccttgttggtaatcaagctctgactcccaagaaactgtgcgcaggaggctataatttaaaacaaagttgatgaaccagtcgagtgcctttcttaaaattactgctttaaagtggatatgttgaaattatcagctgctaattattggctccctgacaaatggcattatttgtttttcctgcttggcattttaatattatggaataagcattcaaatgtaaatgtctaataatttgtgtattatagaagacaattccatggattttacagagtgggttcaataattcacccgaacaagcctgggaccggaaagtgtagtcaagcattctgtgtaaaaatttatctccagagtctctgctctgagatactcttgttcccccaaagctaggctaccagcagacaccaccaatgaggaggtgtcttggaagcatagacaggtgtgacgagggcagaggaggagacgaacatctgctttcttcagacctttgccacaaattcaaacaggaccaaaaataacacctttaactcaacgaatctgtttcgaattgtgctggatatagagaatttctcctctcccctttcctgtcttaagaatgccctgttaacatgtattgaatattaaattatttaacaaaatggcatctgacgataaaaggacacctgtcatgagagtggcctgtccccactcttcatgtgtgtttattccacactagacccgagttttaactatggatgaggctggataccagtgtgtatcacagtccaagtctcagatttccactcaaactacctgaagagacattgatttgcttctctgcacctaccattcaggaatataatcataggagaaccgtcaacaaaaaaagccaagtaaagataaatgctgtgcattgccttgccctggctcacctgccactgtgaaaggggtacagagtcactctgggctcatttgaattacttcctggtttgtgtctgagttgtgttgttattttttttttttttttgataccgtaaaaagaaagttatgctcaaggaagaatgccatagttaatgtgtttcttcagcaaaccccacagagtcagagtgtgtgaggccttcagtttgaaaaggtcatttcctcagatcttagaagccacatcttttagaacccacagtctcattacatattccatttcaaagagggacttctgaccaccattgagtagatgttggagaagaacaaagttacttgaaagatctttctagtaaagagcccttatgagctataagccaaaggggatggagatgtcaactggaattttaaaaaaacaacaaaaaccttaatgtgtttggtttctggttccctcggcctctatggaacagctaaagagcattattttggtttctgaggttaaaactcttgcattttcctcaagcatggtgtttatggtttgagggagaagactggaactagggaactgagggcagtaatcttgaccccctctctctctctctctctctctctctctctctctctctctctctgccttctgattctcttagaacggaacatcttctggtcttccaacatgagaggccatagagccgagtcagtcactagaaagcagtccgtccccgggactatttcagtttcagcacttggatgactgtaaacgatgactgtcacgaagcttacggctaactgtggaggcatggatggtgccgtgctgggttatcaattgacagttcaattcactcagctagctagcttgccatctcagagtctgactaatgacctcatattttttttttcccctttaggttattggcgtggtaagtaaggagtacataccgaagggaacacgctttggacccctcatcggtgaagtctacactaatgacacagttcccaagaatgccaacaggaagtatttttggcgggtaagtaagaaaaccttttttttaagacttttcactatagggggtaaatggagcttaaagaacaggctcagttccctttcaacacagcagggctcacccagggaaacactggaattctgagcaagttccctagaactggttaaacgctctgcctagaatattagctggaggtggttagatgtggactacctgtgaatctcaatttttgcaactcattgcatacaggtctgggagggaaggaaacagtttgccgcttgcttcaagggcaggctcatttgcatttctcttcgaggaagtagtaatgagtcacggagacttacatttcaccctttcttgatttcttgctgagttaacttcatttgaatggaagagttatcctgagtgaacttgatgtcgaagacaaatgtcactaagagaggtagatggtgggttagtggaactgggaaggatcatggagctagttttatttaaatccatctgcaccataaaacggttacatttgacagtatcatagttgttaagcatgaggaaaatcctcgtgtgcctatgagattgtagactcaggtagaataactattctaaaggtctggcctatgctatctcctttggagtgtcagggttagcgaggattctgaggtgacctgggagatgggattcatgggtaaaaattgttctctgagatgtctctggcatgttcagttttcctcagtgtagaaatgaagagctatttatacaaatttagtgagctgttttcctcacacagacatgaaatatacaccacccagagaaatggtaatatccacaactggattacatgagaaaaagactttggttaaaaaattacttattcctgagagagtctgtcttcagctaggaagtctttgtttccagaaacgtactacttctacaactgcatctgtagtcttgttaagtatttgttctcaatttttatttatttaatacttagttggtgttaaattatattgctccaagatcgcagtctgagattatgagcggtttcgcccttattgctgttctcagatgggagccaccagtggtagattaatcttggcctcagctggtatgaatgaagacaatccgacactgtcgttttgaaaacggtcaagaagcaccaaaggctctcccattactgtccccactgtcccatttaaagatttacaaaaagaattagactaaataactagaaggcttcccttgggaggaattatttaagtcagttgtccacatgcaaggaagacaggaatgaatcttttcacaggttggaagatcctgatttgtcaagcaggaataggaacatctctgtgttgtgaggaatgaaaggttgtcatgcaaattacacagtcagagatgctcaggttgagaaagcagtgacatttcttgtaactgtagtatgaatcagcttgtgtttagtcttcttgatactggatggaaaggctggtataagtgtgccttttacaaaagcatgatgatagtttcttggggtgcgtgtgactttcacgacatccaaggtccttttttttaaaacaaggatacagtaaaccgtagccatgaaaggcctactgggatcggcacaccctctgctagctgtttccaccctggtgtaagggcgatggaaccccttgttcctggaagtttgcgcgtcagagtaaacaaacttgaaaacccctcttgatagcagaatccagtcggtcttgttacattttctcttaacaagatacaccgcggaagctctcgcaggctgctttgatgaagccacacgcaccccccacacacacacacacacacacatacaattcacaggaagtctctcttaaaagaaactgattctgctgtttactgcctgtgttaaagggacagagttccttttttatttctgataacgttagagggaaatacagaaacgttcacacagcctgtgtgtgactaagaatacagcaaatagccctgtagagcaaactccctgaggtgagcatggaagcgccgtacctcttggagactgtcttgtgtgtaaagagggctctctgctctgaaggaaggcggtggttcttcaaggagaggagcttttctggagaggatgaggagtgagtctttgaccccttggtttcagtaggagtgtatttctcccctgctcttaactatgcctttaaccaagcactctgagtacagctgtgagtcagaggtagcattgctgaagaagaaccatatattttctttctcttctttttccaaaaggccaaactcgtggctcttgtgtgtgtagaccgtgtatgccagcctcctcacagatatgggcaaatctctctactccttttcaagagagaggcacagggtggccgcctgtgtttaccaagaggaaaagttacttctcgataggctgtcaaactttggcctccgtgccagtgcctcactctgttatggcaggtgaagttcacctttgccccacccagtgtttccacaaaaaggcagggttccaagtattcatctgaacaagtgttactgtgggactcagggttgggggtggaggatgtttgcagagcctaagccccgggcgggcggaggtgtaggaaacacaagtacagaggccatagaaaaaaggtgagactcagtttgacgcagtcctctcggctgctgtgcccagtgactcaaagcactagaagtcagcagagttggaactctgggctgagcagagtcgcctgatcgatattcgctactgtagcaagagtacctctttatggtagtttcacccactctcggctgttgttaattggaatattattattattattattattattttgctatccactgccctccccaacatgagaagaccataaaattgaaatggaaaggtaactagcacaatgtgccctgtttcctcccccatttctgctgattcagcgtgagtcccaccggatcagcaatgaggcctggagtcatgggtacagcgttggttgctcgcctgtgttccttctgagccattcagggaagcttcccggtcgctttgggctggccggctgtctttcacactgcatctatcctctccttttgaacagatctattccagagaggagttccaccacttcattgatggctttaatgaggagaaaagcaactggatgcgctacgtgaatccagctcactctgcccgggagcaaaacctggctgcctgtcagaacgggatgaacatctacttctacactattaagcctatccctgccaaccaggaacttcttgtgtggtattgtcgggactttgcggagaggctccactacccttatcctggagagctcacagtgataaatctcagtaagtggattccagaccaaaaaaaaaaaaaaaaaattaaaaatgctagtaatgtcagttctgcccctgtgagctaataacatgttgtctaattatacggcttcgtcatgtgttggactaagtaggtggctttagctaagacgaggaagaggaaaaacattctttaatgtccctacttcttattataaaacataatcatcaaagatatacatattacatatattgtataaaataaccagtacagaatgttgttttcggaaagttgcaggaagaagtatatttccgattctaatttatgcaagcggctgtaggcacaatcccaatgggtatggacctgtggaacaggccagctgcagtcccttcctgctgtgctgggtcagagctttgagttttcactgaaatcttgtgaagatacgtgtgcctgtaaagccatgatctaatgtggaaagctgttttctagaaaaaaaaaaaaaaactgtcataattgttcaagtagtctaagtgaataaccctaagagatgtcatatctgagcttccttccttatggtaaaggggactgatctcatctttcaatcaggcttacggtaaccgcctatctctttatcttgacaaattcttgcttccttgggtttataagcttttactttttcttttcttctttttaaattttggctaaagttactgtcatgccctctagcaagctttccctcttcagtcagtctcaggtagctttaggaacaatttaagaataaaaaaaaaaaaattctaacttctattttaactcagggtgtgtgttctgggttatactgccgaatcttacagcactttttcaaatgaccatcttcccatgaaagctaaatgttgaaggtttaaaagttttcattttaatagtctctgaaatttgagtaaacatttccagaaatctatagagagttccaagctagactttacccagtttctactcttcagtctcatttgctttccctggagactaaatgtagttcatattttaccactgaagcactagaaatattaattttagtattttaacttttaagaccaaggacagtgtctcgctagccatgttcattctacaatgcttgtgctctcaggaattttcagttttctgaaaatcttagcttcagtacctttcctgtaggctcacaatatagtgtttgtgcccgggccctcagctcagcaacgtacacccttgagctaagcatggtgggtaggtacctgcccaatagcagcaagccctctcctcgtgtttgggtttgctttgggtgtttgtttgtttgtttgtttgtttatggacaaggtctcgtgctgcttaggttcagttcagacttgatgtagtcagggaaagccttgagccactgtcacctccaaaatgctaggatcacaggcctgtgccctccccccacccctgcctcccaccatccccccaatgcctgttccgagtgtttactctttgtgtccagaagtaagtttcattatgctatgaaatgacagctttgctcttcagacacccccccccttttgactgatgcaggagtcttctgagggtcacaggaacacctcctttgtctgacattcctaggacagaaagagagttaaccattcagctgccgtgcaaggctcttgctcctgattgtgaaacctgttggcccaggtgtggccactgatgactgacactctgatcaggaaaatttccagcatttcatcaggcctaataggcagatcgagtgtccaagatgggctgtgctagatttccaggcttaaagcacaatagaggtctgtccagaatctccgtaaggacttccatcatggggtgcaggggatggaaacctaatgaaagaatgtaagtccccagaaatcacaaactgacaggaaagagaagggagagaggaatgtggaaaagaactgttaaatttagctcctggccctcccaacctttttggtaatttttttttctatctatctaactaacccatctagaaatcagttgaccaaattatagacttctgaatgttaatctgctttctcggtttcagttgaaaacagactttgttttgcctactgcagaacttctaggttctttcttgtagtcttaggggtgcttattatagatcgaaaatgtgagtcggcataattaagccattcagaaccttccaaagcagctcactcttgaaatgactctgtccgcctacagccatttaagatttaagaacaaaaacagatcttgattttctttttcatgttagctcaagctgctaagtgggagagttagaaatgatatcagctcctgtgattagtcagctgctgaaggatgagtttttaaaaatgtaccttcatatacagtctataatttccagctgtaaagtattttagagactgacattttgctgcggatattccttcaggataagttctcagcctggttgtttgtttgtttgtttgtttgttttctgaagacagagccaccaaacgctaaattatgcatgtcacggagaaaatgaaaagctctgacttcattgtttcttggttcagtcattagcttcacagtagttcagtaactaaagtgcttagcaagaagagagccgattaaacctgtgctctacactggaagaaagcccaattctttatacttaacagctttcatttgttaagtttccactgtgggactactacaaaaacattatgagtctttgatgtgatttgccacattaaaaaaatggcacagacaggggtgtggtggcacacaccctcaatcccagtgatcgagaggcagacgctggtggatctctttattcttggccagtccaatctatataacaagttccagacctactgagactgcacaataagtcttcccccagaacccccatcaaaaaaagagcagagttaggaaggccgtacacaagcaggcttgcacactctcacgcgctcgctctcgcgcgcccacacacacacatacataggcatacgcacatgcgcacacaacttcttttctcttaacctgagggtgcttctaaaatcattatctttttgtcttacctccagtaaatcccttcctgtgactgtgggatgcctcgcctgtcagccttccagcttaacctgtctctctcttcctttaccattttagctttaaaaaacaaaagtgacaatttgaacttcctgcctgctgggcctcacccgaaggactgatattgggctgataaggggatatttattttggttgagaggcttgagaaattgctctcccccagaaagctttctgtcactgaccccatcaacatctcccctgatagtgttgtccacggtgtttattctggggctctggcttacccaggagtaactgataacagccagcaggagataacgtcctgtaaagcgctttccgactggcatcaaatccctccagcctgtcagcctggagaatggatctgaaagctttagttctgggcttccacagagttcatcttcagacctatcaggtagcaagcttggagttccttctcagttaagcccaaaagggctgttttataagagcacaaaggatacttctttacattgtcttaagtgtcattccaaacctgccagatcttggaggtcaagaatcttgtttctactccgagcatgtgcaccccccaactaatgatgctctcagcatcctggggagaagtgcctgtttgaatgagcatcccagaaacacaactcagcctgtgcatcggatgtgttttatctttggcccaggaaagctgagctgaggcttttcctgcgaaatagggctacataactatggacagtttaggacagtattctccttgtctgagcttgaccagggcatatatgctgtctctaggagtaaatgtttgtctcttagctgcctctgtcttctttggtgctgtaagtaattgaactagcttgggaagtacctgtcgtggtttggcagaggtgactgtcacacctcacgattccaggagacagcccagatggtagtctggttagaccagaaccttggtgaaatgctcgcactgccgagcaatggctagaaggggcagccgccatgcccttctagttgatacaggcaattcgaacagggctcatgaagttcctatgtaaagagaatcgagttggaaattgatgacagttcattacttaaaactagtcttaatctttcatctaagtttgcacagcactctgatttcctctaggtaaactgcgaatgacttattaacccgtgacaaccccccaccctgtattttttccaccccatcttagtgaacgctctgcccgttccagtttgaacagcacttttctatcctagttctcactaatggaaaggagatcatccaaggggcactgggctctatggaggctggcattgtcccagggtttgatgttattcccgatcccccccccccgccccccggaggaagtggagcagtgtttctgagtgggtggcccagagccctccctccggagtgagaggcgttaggggccaggtgtctagcctttgtatttgctgctgctcagggtttctcaagaagagagaatggctttctgatttcacttcagttctccacagccctgtgagtaaccgccctttcttcttcattttagcacaaacggaaagcaacccaaagcaatacagtagtgagaaaaatgaactctacccaaagagtgtccccaagagagagtacagcgtgaaagaaattctaaaactggactccaatccctccaaaaggaaggacatctaccgttccaacatttcacccttcactttagaaaaggacatggatggctttcggaaaaatgggagccccgacatgcccttctaccctcgggtggtttatcctatccgggcacctctgccagaagactttttgaaagcgtccctggcctatgggatggagagacccacctacataactcacagtccccttccgtcttccacaactccaagtccccctgcgagcagcagcccggagcagagccttaagagctccagcccccacagcagcccgggaaacacggtgtcacccctggcgccaggcctcccagaacaccgggactcctactcctacttgaatgtttcctatggttccgagggcctgggctcctaccctggctatgcacctgccccccacctcccaccagctttcattccttcttacaatgctcactaccccaagttcctgttgccaccgtacggcattagttccaatggcttgagcaccatgaacaacatcaatggtatcaacaacttcagcctcttccctaggttgtatcccgtctacagtaacctccttagtggcagcagcctgcctcatcccatgctcaatccagcttccctaccgagttccctgcctaccgatggagcccggaggctgcttccaccggagcaccccaaagaggtgcttatcccagcaccccacagtgccttctcccttaccggggctgcagccagcatgaaggacgagagtagtccccccagcggatctccaacggcgggaactgcagccacgtcagaacacgtggtacaacccaaagctacctcatcagtgatggcggcccccagcactgacggagccatgaatctcattaaaaacaaacgaaacatgactggttacaagactcttccttaccctctgaagaaacagaatggcaagatcaagtatgagtgcaatgtctgtgccaagacgttcggtcagctctccaacctgaaggtaggtctccagacccccgcgggtttctgcccacagacccgtgctggtttgcccttggctgccaggcagtgcattgttgagtgcacttgagccataggagacccagcttgagcctgaactggggtctgctgaatactgaaaatacagggtttatctcagtgtctttcctaagaggcttgcatctgcattgtacatacctggctctgggaaacctagcaggcaggcaggcctcattgcaaccccagagttcacccctgtggtttcttcccaggtccacctgagagtgcacagtggagaacgacctttcaagtgccagacctgcaacaagggttttactcagctcgcccacctgcagaaacactacttggtacacacaggagagaagccacatgagtgccaggtgggcagtattctctgggtagaactcttgacctctgtggaaaagtagctgtagaattgtcttcctgtgttgtttcaacaatacaaaaaatatggtcttgtactaggctgctggccctgcacagctcctgggtactctgtgactactcacaggctatactgaggatggctgggtggatgtcagtcaagtttcagtgggtggggacatgtcctcanataaacagtacctcagagtactgtgtgcccagcttctccccccccccccccccccccccaccgcgcatgagcattgttaagaggcttctggtctcccgaggtttctggctattggcctgcctttccccntccagctgcaaacaattaatcttggtcttcccntgtgccctttctctgtcttcccttgccctcacactttaggtctgccacaagagatttagcagcacaagcaatctcaagacccaccttcgattgcattctggagaaaaaccttaccaatgtaaggtgtgccctgccaagtttacgcaatttgtgcacctgaagctgcacaagcgactgcatacccgggagcggcctcacaagtgtgcccagtgtcacaagagctacatccatctctgcagcctcaaggtccacctgaagggcaactgccctgcgggcccagctgctgggctgcctttggaggatctgacccgaatcaatgaagaaattgagaggttcgacatcagcgacaatgcagaccgtcttgaggacatggaggacagtgtcgatgtgacctccatggtggagaaggagattctagctgtggtcagaaaagagaaagaagaaaccagtctgaaagtgtctttgcaaagaaacatggggaacggcctcctctcctcagggtgcagcctctatgagtcatcggacctgtccctcatgaagttgcctcacagcaacccactacctctggtgcctgtaaaggtcaaacaagaaacagttgaaccgatggatccttaagattttcagaaaataagtgtttcgtgttgcttcttagggtatggcttggtgaatcagggtgcctttagcaaattgcttgtacatgactccagatctgcaaagctccgctggcaccgggtgcttccctgcacctctctggaattaaagaaggactccaatgttaccaaaatctcagggcataaatgaggcaaagactcactatatatacatatatacatatatacatattataaatatatatatacttatttacagccatgtctatatatttgaacctgtgtattttgaatatttgtgtggatatgtttgcatagcgccttcctattactaaaactattgcctagccataattattttttcaatgataattcttcataatttattatacagtttatctttcaaaaagcaataattaaagaagtttacaatgactggaaagattctttgtaatttgagtataaatgttgtatctttgtcctgtggccattctttgtagataatttctgcacatctgtttaaatgcctgagacttagaagatagctctgtgatttcaggcaacctttctctatgataatgctttaaaatgaggttttgatattgccaaagtcatgtggttggtgtgttaactcagaagatcacacaatctgagtgacattctctaagttggggatacatgtgcagaattgctcagcaataatttgaggggaaggaagaagaaaaatattttatgtttcagaatgatggtttggttttcctcctcctagtcacaattttaccaaacagtgacaggaaggctttgccaacctgtctcccaatgtcacatgaccattctgagtggccatatgactttggcatccctgggtgttatctgaaaatgtgaagaagataaaaaagccgtgttcagaagatctgtcgtaaagcacagatgttgtgtgtgtgtgtgtgtgggttggggggtttgagtctggctgtcattttgctgttggcttgtttttgtttttttaatatcaaaattgcacaaagctggtgccctaccaagaaggatttgatatagaaaggctcaggccacacttaaaatacaagcaagcaaagagaacagaaaaaaataaaagtaaaaacgggtattcttatcatcttaggttaagcgggtaatgaacactcctgtccccaacgcatcaactgtattgtatctgtaaaactcagcttttctcagtattttgtttttgcattgtataattaacttaattaaagatgaaagggcattgcaaaagtgttcaacaattacctcattgagtgtatccagtaggagtgcaggaattaatgtcgtatctcatgagttgctacccagctgagcgtgtgtgcttccaaatggtaggctgggtggttcggtcctgtattctcctaagcccaaaggttacctgttggtgttcaaggtgtaataaagaatgctgtatatttatgaacctatttataccagtataccatgtgtatatatgatatatttataaccacttaaattgtgagccaagccatgtaaaagaacctatttttcctaagagcaaaaagaatctctctgaagttttgcttaaaactccatgacctcgctatgactttggtgcttgggcaccaccctgcctactaccagagagcagagcacctcagtgcagaggtgagggtgtgtagcatcttgggatggatagaaacaccacaccatccagtcgcattt

SEQ ID NO: 6 - DNA, 23615 bp, human
gggaagccagacggttaacacagacaaagtgctgccgtgacactcggccctccagtgttgcggagaggcaagagcagcgaccgcggcacctgtccgcccggagctgggacgcgggcgcccgggcggccggacgaagcgaggagggaccgccgaggtgcgcgtctgtgcggctcagcctggcgggggacgcggggagaatgtggactgggtagagatgaacgagacttttctcagatgttggatatttgcttggaaaaacgtgtgggtacgaccttggtaaggaacttgaattttttttttttaattctgaaattgatctgaaaactttattttcttttcctttattgttattattattaattttttttggctaatgtcgcagtagaaacatgcttctgctttagtgcacttagtgctgtcaaacatttgtgagactttccttatgaatcattaaccctttcagttctagacataattgccaattcattgaaatttcagtagtggttccagctcacactcgtcaaactattccgggttggctgaagttttctattttattttatttttaacatgtgtttggcgtcatgactctacatgttggaactaaataaaaataagcaggtttgcttaaatcataactgagggaaaaacaactttgcatccaacttttttttttttaagagcatcctatttagagaagtggaagaatgtaaaaacctccttgaaggacttccacagaatgttatgtttacatttgaacaaacacacattcttacatggaaatgatacccatattcctcatttttatcaaacatgtctatatgagaaaacccttacagaagttgtttacctttttttgcctttggaaaacagtttttttctgagtgtgagggaggattttgggggaatatcctcatcaatgtacaagtggaagcagagcttgtcctccaagtcttctaaatttgttataactttagttacagtaaactgtagtacatcagtgacttctgggaattcatacactttcagatttaaatggaaagtgctatttgtagctgaggactcctaaaggaattctctccagggaattttattaaacggttttatgttttgtttttgccttttcaatttggtatgagatgcttgcaagtcagaagacactgcaggctgttttccccttcacccatttttcctcctcttttcctgtggtccaagtgatttctaagaggccgtagctcagtgatgctggcaggatgcaacccttttcagtcttccatgtgagaggatgaaagagagctcgcagcagagggaggaggcagttgttttgaaagttgttttgcgttgggagctggtgggaaagttcggtcttccccatttggaaaaggcaggctgggttccgctcctgcaccacacgcgctttccatttttagcttcattcagaggcagacagagctccttcctcttcctctccttgtttgtgacacttttctgaggcagcttttccacagtgccgagggtctggcggccatgaccccaggcattctgggacactggactgtgtgcccagaacatttttctgccatgagaggtaaagccagggattgttcagaggtgattctttttttttttttccttttccccacagtgaggttgccacattcttttttttttttttttttttaactaagagtagcatttaaaaaccttgcttcttttcaaggcagtttactttatacggcttcttggctctttctcaactgtaccaagcactctgcatctgcttttaaagtcttcagactactgtattagtcatagcctctcagaaggagccacaggaacggcgggacaatggggattaaaggcctttcctttctcttccaggctgcccccaagtgtaactccagcactgtgaggtttcagggattggcagaggggaccaaggggaccatgaaaatggacatggaggatgcggatatgactctgtggacagaggctgagtttgaagagaagtgtacatacattgtgaacgaccacccctgggattctggtgctgatggcggtacttcggttcaggcggaggcatccttaccaaggaatctgcttttcaagtatgccaccaacagtgaagaggtaagcctctggtttattgacaagaagattggggacctggtgccaaatctccctacttgcccttgaggccttgtatatctctgaaaacctctgagaatctgtaagtatcagtaaataattgattgctctattcaattcttgcattgctttctctttccctaaaccatttccttctcatttcttccagccttcaactgttcctcactaattagtaaacagttaaatattttggcaaattggcatgtcttagaaaagcaacttgcagcatagggtgggtgaaattgtcagtgaacttcaagaaagctctgggcccactggccctagtgtccctgttgtacaatatctcttaagggagaaaactttttcttggaaaaaaaagttttaatatttcttttgtctgtctttggtaattagttggctttactctttttaacaagccagctttattagctgggtttctaaaactatcctcaaaactttgacgtgtttatgaagtgaagtgatggtataagccgagaaagggggtcatgtaaaagtgtcctctgtctggatgacttcagagctaaccactgtttatctgcagcagctcctttgctggggctgggctggcaggccaattccttactggcctctgaagtgggtacactctttgttgtttcaaaggggatgaaaacccaaacttggaatgagcaactgatttgtgtttacctttaatataaagattatagctaggcgtgatgtttagctgtgaaataacttgcagaaccacctcttgttttcagaatttggcttactgtagttgcacttactagtctactactgggttgagttgaatgaaaggacccctcagcagacttcccataccaccattagcacagagcactgtcaattctattacttccttcagaaagtagggaaaggggatggttggccgtagaaatccagaatttgctactgggactattccttacagccgtgtcaaatcactttcccccttccctaaggaacagtatttgaaggaatccgggagactaggaaatgctttgtctctttcctgtttgtgtgtctgagtccagtgtttttggtgagggggagacacagctgtctaaaattaaatagaacaacgatgctcagtctttcttttttcccccactgctgcgtgaatggaggcagattttataaaaaataaaatttaaattctagttgtagttgaatgttaccaaaaaatttaaccccagaggtcagccttaattatacctcttcctgaaagtaatgctcttcttatagaaagtctctcaatatgtggcccttggaatgaatgtaggatttgtttgggtatatgaggtttgactatggctttaaaaagtgtagtgtgttttttccccatatcaagtttgaccaaatattcgtctgttcccaagcttcgtttgtgctgtgttgccctgtccagtcctttgggactgaagtaaaaatctccattctgtgggtcatctttgccttttaattctataatgttcttatcgtcagttcagctcttagcttaagtttttcaatttccgtcattctcctttaaaatcatagctaagtcaagtaatgctcttagatcatttagttcaaggttcagaaagacagtcacttacccaaaggcatataggtaaagccagaccggggacttgaagcaaagcttttctgacttctccaatgagtgcttctcgattatgctacactggcccctcttggtgtcactgtggttttagttatcatcttaatatatttcagaccaatccttaagctattttgtgtttttttccctctgaatgtataacttcctatttcaatatattaagcaaccaaaaaacagacaatgcctgtgttcctgaagaaggtccttcgaaactaacctaagaggtgagggttctcttatcctatttccctatcttgcatttaactgaagcgcttgaatagaggatagtatgtctttaaaccctaatcataaagtcaagcaaggagtaaacttgaatgtaatactgtcttaagcttactctggaatttgtacctgtttttctgtttctctcctttgtttttgaactttgggaaaggacagcttcctttagaaggtgaaagaggatggtggagttgagccattaagggtaggatagctgcaaactagttccgggacagcaacattaaggattgatgaaatgtttacattatctttattcagttagttctttaagttccatgagccattgaaataggaagagtctttctacatgtactttttatctgacttattaatcttgcccttctttaaatgttttcgtttaaatagcaaaagatgttacaatatgtactattgtgctgacataccggggaattttttttttacaagaccatttttcattatgaatttgtaccatgtgtcagcaccttttttttttttttggttaaaacaaaacatacaaatcatatttctgatcttgaccctcatttttagattgggtttagaatcattctgcacctttaatgaaaaactgttcttacttcctatttccagaactgaaatcaaatatagacctactattcaaaactttgaaaatgttttaaactgatgggttattcttttatgggggttttccccttgtcctcctgaaataatctcttatgactacttgactacagaaaaatcagttcaagcatttatattttagaagcgttaccctgtagagtttctgccgtcggttgagactgcttgcactcaagaggcactgtggctctgtgagtctcacctccctgtatcgtggctgataaacatcagacagattgtgaggactcctttggtgtccattctgatcgaaatccattattaactcaagccatctgtaaaaagtatctcaccataacatgcaattctctcttcatgttgccttttatttgagaaatgatgtaccaaggcatcaccatgtctcctttaatagaaagataatgaagaaggaaaccttctccaaacaactttgttcacttcactccttttaaaaatggggtgattgccgggcgcggtggctcacgcctgtaatcccagcactttgggaggctgaggacagcagatcacctgaggtcaggagttcaagaccagcctagccaacatggtgaaaccccatctctactaaaaatacaaaaattagccgggcgtggtggtgggcgcctgtaatcccagctacctgagaggctgaggtaggagaatcgcttgaacctgggaggtggagtgagccgagatcgtgccattgcactccagcccaggcaacgagagtgaaactctgtttcaaaataaataagtaaataaatgccattgcactccagcccaggcaacaagagcaaaaccctgtttctaaataaataaaataaaataaaaatgaggtgattgcaggctgaaggtcttggtcactactgagttctagttctctcaagacaaatattcatatggtcaaaacagaattataatctaacgagtcattcttatagagtgggattcggggagggaaaacagatagctagtaatttaaatgatcttaatgtaatttggggagaggatgatcagatgacatttagacttggatatagtttctgcttcaaacccacgctgtgtgtgtctgtttgcatactctctaaagaaggaatttgaaaaaaaaaaaaaaaaacaaacctatattgtccttgttatcagccttccattaagcgctccaactcaacccatcctaagctgtcaaatctccttcattgtgtaaaattgaaaagaagccctgtgttttgtccggaagctgtggaaactgtggacggcggggtgtgttctgtaactgaggccacagacagaaacaaatctgctcaagtggtggaggggtctgcaccgcacataaagagcttctgtgggagcaggtgaagcaaggaggtattgaagccgggtgtcctcttgctgttgagtttatagaaggcacagagcggcctgtccccagcagtaattctttaggaccctggtcagttccagtgaagtttccttcttgagttaggtgaacacattctttgtctttttagcactgtgaagtttaagctacacaccaagttcctccttagtatttgcttggttcaaagataaactgttacaaactatgggtcagggagatgggtagtctaagatgcacattctggatctttctaggaccagagggagacttagtacttggttttttcacttccctttaaaaacaatcctggttcctgttccccctccaaagttacctggacaagcaccatagtcttagagtttcagcttttatggcgtctgtgtctcagacgtgcatttgagatgaagatgtttactgtatattagaacgctagacttagaatggatataggtggtttgtaagtgccttccagttttgaaattttatgattttgtatctttatctataggtgtgagtttatatgtgccctatagcactgtatgtaattgaaatttaaaatattagggcgctctgtccctttcctaatgataagctaatattgaagctctttaatttgtctggggtttgaggattatcatagacctctcttctaccacagagccttctctttttaattgttctcctgtcttgcctgttgaattgaatttgtaaggttagagatggcaagagctacttccggtgctgctttatagcttactagtgatgagtctatagttgtgctcaaataggtattgggtagcttatgctttctagttgacttctttttgggcacaagaaaatgtgtgtaagatggataactgaaaacctagaggcccattctgtaaaagaatggcaatttggaagaagaaccacgacctcagtgcagtgctccgttttggtaatcaagctctgactcccaagacaccatctgtaagagactataatttaaaacaaagttgatgaaccagttgagtgcctttcttaaaattactgttgtaaagtagatatgttgaaaatatcaactgctaattattggctcaccaacaaatggcatgatttgtttttcctgtttgtcattctaatattatggaataatcactgaaatatgaatgttaaatgatttgcatgtcatagaattcaattccattaattttccaaattaattagattcaataataatatgtccaaaacaatgagcctctgaataagtctactaaagctttaaatatatatatttatatctcaagactgctctgagatactcttgtccccagtagttttactgccaacaaataccaataatgacaaacggtgattaaaagggcagatagatgtaaatgaaggcagaggaggaggatgaaatgaacatctgcttttttcagctctctgacagaaattcttaaaggaccaaaaataacactcttgtttcaacttatgttaggtgtaggaaaattttctttttttctctttccagtctgagtatgcttcattgatttgtatattgaatactaaattgttttagaaaaaaaaatcattggctgataagacacttaaatgagaaaagctagtctgtcacaggcatgtaggtgtgccatatctatttaaatagaggtttaaattattgatgagacttaatgaggaactggtgtatattgtaaccaaaattgtagattccactgtgaacaatctgtgcatggatcgattcacatatttgagtctaccatttcaggtacatgaatgtacaggagctactgggaatataaaaactggaaaataaacattatcatctattaccactccctcctccttggtccattttagtaaagaattcagtaaagtgataggtaggctatcttgaatgagaagaaaataaaactaggaaagtgagagaataaaaaccaaaacaaaactgtaactgcaaactgttgtaaaggactactcacagaggagttctattgaatagtgacgaattgcttcctgattcacggttggtgatttttttaccccttcaaagagaaatttattattaaggaggaactggtatagttaacatgtttctttagcaaggccccacaaagttaaaatgtgtgatgcatccagtctgaggtcatttcctcagatcttagaacctacagctttcctccgtataaacttaatttcaaaggagggcttttggccaggcatggtggctcagcctgtaatctcagcactttgggaggccgaggtgggaggatcgcttgagtccaggagtttgagacaaggctgggcaacatggtgagaccccttctcaacaataacaacaaaaattagctgggcgtgatggtgcatgcctgtagtcccagctactcaggaggctgaggtgggaggatctcttgatcccaggaggtcaaggctgcaatgagctaagatcaagccactgcattccagcctgagtgatagtgggagaccttgtctttaaaacacacacacacacacacacacacacgagggcctttgaccactcttgagtagaagactcgagaagaacaaagtagaaggccagagaagaacaaagttacttgaaagatctcttattaaagagaatgtacaagctatgaaaaaaaaaaaacacacacacacacacaaacctcatctggaatgaaaaaaacataatgcatttggtttctggttccttaggctgttatggaacaaccaaagaacattattttggtttctgaggtcagaactattttattcccctcaagcacactatgcttatggtttgagggagaatgagaaataggaaactaggaacaggctgaaatggtctaatcttgaccatctaattctgcagtgtcttattctcattctaaaagagaatggttatattcgctgttctagcataaaaagtaatgataaaaataaaagatcccgtattaccagacaataatcccctagactgttttaatgcttggttgagtatttgcttatgatctcagactttaaaagatggtctccccctatggtgaagcttgttaattatgtaggcatcattaatgtctgtttacttatcaaaattttatcattgttagttgtattactacttgacagtccaatttatttaattgaaaagattggttaacattttatagtcaaagtaattgtttcctgtgttttttcctgtttaggttattggagtgatgagtaaagaatacataccaaagggcacacgttttggacccctaataggtgaaatctacaccaatgacacagttcctaagaacgccaacaggaaatatttttggagggtaagtaagggaaatttcttcagacccattaaatgttaggaaaaaatggagctaaaagagctgggtggctcacctttctcatcctgtgctgagaaatgctggggctcacccataagtatccagcatccccatggacacagggaattctgaacaaatgtgatgaaaccgatgaaatgtctggcctgtaggtggttagtgatggagatacgggctatatgtgaatcttgatttttgcaattcattagagctttgtaatgaaaggaaacagtttgttgcttgctttaaggataggttcatttgcatttctccgcaaggaagtagtaatgagttaccaagccttagatttcacccctttttgatttcttgctgacttaactttaattgaatggaagagttatcacaaatgaattatctttttggtttttttttttttgagatggagtctcactctgtcaccaggctggagtgcaatggcatgatctcggctcactgcaacctccgcctcccaggttcaagcaattgtcctgcctcagcctcccgagtagctgggactaaggtgcgcgccaccatgcccagttaatttttgtatttttagtagagacggggttccactatgttggccatgatggtctcgatctctggacctcgtgatccgcccaccttggcctcccaaagtgctggaattacaggcaagagccaccgcgcccagccaggaatgacaaatgaattaccttataagtaaatgccattaaggaaggatagctggaagatgggttgaggggaatggaggaccacagaactagtcctatttaaatacatgtgcatggtaaaatgattccatttgacaataggttaattatctcatagcataaggaaaatgcttaacagtcatatgcaagatgataagctttcctatagcatccaaccaaaagatctagccagtacaatttcctttgctatattagggttagaaaggcccccagaggtgaaccaattagatggaatccttgaataaaacactggattagcagtgaacagaaaaaagtcagattgctttccttcttcccatagatgtctcagggatatttagtttcctcagaagataaagaatttagtaagcgtttttttgtgcatacttacatgaaatgtacattatttgaattctttaaaaagaaacagctgcatgataacaaaaattgtgttatgcttgctttagctggtatttttgcctagaacgattatatcgttcggacaagaagctattcctaagaaacaatatttttaatccaggaagtttttcatttttagaaatttatcttactatttcccaagcaaaagagggtagttacagattcactaagaatcatgtgctcacaatttttatttaataattattcctccttaaaatatattaatcacctgacttacaatggtggaaccatgagtgcatttttgcctttattgtcaataacgtcttctcagaagtgagccacaaaggtgcatagttcttggagttaaaggtctgaattaagacaatccagcataagtctcattaatgtgtgattattttgagaaaaggcaagaagtacctaagaatctccccctcactgtccagttccctgtttcatttaaagattcactgtaagtaactgaaaggctttccttgggaggatttatttgaatcagtctttcacatgcaaaggatattgtagaacatctcgtttttgctggcaggaatatgaacatctgttgtgaggaaagaaaaagtttcatgcaaattacactgccaaagaagggatgttcaagttgagaaaccagtgacatttcttgtaactgtactatgaatcagcgcattttaatcttctagataatatatggaagtgcaggaaggtggtaggaaacggtgttcattttacatatgcgttattttattctgtgtgagtgacttcatggcaccgacattgctgtttttaaatgaggatacagtaaattgcagtccgaggaaggctaactggaatcaacatacccgtagctttagaaagcagtttccgcaccagcgaagagtacaagagcgatggaaccccatgttcctggaagtttgcacatcagagtaaacaaacttgaaaacccctcttgatagcagaattcacccagccttgttccattttctcttaacaaaacacaccgcaaaagctctcacaagctgctttgatgaagccacatgtatttcccccttcacaatttacaggaagttactcttaaaagaaagtgattctggtgtttaccgcctgtgttaaagggacagagttcctttttatttctgataacgtttgagcgaaatacagaaactatctgtagactagcatagtcggtacgtgagtaaggaaaagcaataacctgctgtccggtgagcacaaaattcctgctacgaacagtgccttactgctgcttggagactgcaagtcgcagatcacactaggtattgactgattgtataaggaaatttcttaaagtctaaagtaaaggtggtacctcctaaaaagaggggaagagagaaaactttgtgtggaaggataaggagtgtgtttatagtttcagtaagagtgtacgttttaatttttcttcttcctctgcctctttgccaagtagcctgagtgcatctgttatccagaagtagtattactctaggacaaacttcaaattcttcattctgcgttgcctttaaggaacaacatactttcttcctgttctttttccaaaaacacacgcctatggctctgtgtgtggtgttttagccagcctcctcccagataaggggttcccttccctcctttgcattgaaaggaaagtgcaagtctggacatgtttatcaagaggaaaagtgacttctcagtaatagactgtcaaattcgggctgctgcccgagtgttcgctttgttatggcaggtgaagttcacctttgccccacccagtgtttccacaaaaaggcaaggttccaagtattcatatgaacaagtgttactttaggacttggagggttgggggtggaggatgtttgcatagttgaagccttgggcgggggtgtaggaaacggcgagtacagaggccatagaaaaagctaagactcagtttgacgtcgtcagccggcttggtcttctacccagtgactcaaagcactaaaagtcagcataatcggaactgaagtcagtagcatcgcccatttgccattcactgcagtagcaaaagtagtactctgtggtgggttaatcggtttgaggcagctccttaaatgaacatttgtgtttcatttttctgttattttcccgaacatgaaaagacgataaaactgaaatggaaaaggtaactgacaaaagtgtgccttacctgtttccgccctgatttctgctgattcaagactattctggctaaactgattggattctttttctaactaggcagtaggggatcagaaatcacacacggtaccggctgtgtttattctgagaggtgctggggagctttgggtctgacttccttttacatgcctgtcttctcttttggacagatctattccagaggggagcttcaccacttcattgacggctttaatgaagagaaaagcaactggatgcgctatgtgaatccagcacactctccccgggagcaaaacctggctgcgtgtcagaacgggatgaacatctacttctacaccattaagcccatccctgccaaccaggaacttcttgtgtggtattgtcgggactttgcagaaaggcttcactacccttatcccggagagctgacaatgatgaatctcagtaagtggattacagaacaaaaaaataaaaaatgccagtaatgtcggttctgcccctttgaactaataacatgttgtttaattatacggctttgtcatgtgttggatgaagtaggtggcttaagctagggactaggaagaggaaaaacattttttgagtccctattaactattaggaaacttgatcatttaaaagtatatatatatatgaggagctaccttgagttttgaattcaggatgttacaggaagaaatatatgtccaattctaatttatccaaaagcagttgggagaattacagggattggtccagacatgctgcgtatgcaaggtatagccctcatctgtggtactttggcagggcttagactgcatcaaaatatttatagatgtacatttgagtgtacagttaggatctgatgtggaacattgtaagatcattgctagaaaaactttgtcataatttttcaatattattctaagtgaataaccgtaaagattttacatcttagcttccttccttacagtaaaaaaactatctgatctcttgatcagtattatagtagccacctatcactttatcttaacaaattctcaattccttaggtttatgtgcttttacttcttttatttgattaaaattgctgtcatgacctctctctgcagagggctgcatcattttggtcattctcaagtgatctctttgagcaatttaagaattgccataagattctaacctctgctgtaactatggttgtgtgttcttggttagaccactaaatcttattagcagttttaaaaattattccttttggtttagaagttaagactaaatgctgaagtttttgtaacttttggttttgatatcatttcaaacttaagaaaacatttgaagaaaaggacaaagaatttccacttaccctttacccaggtttaccagttattgataagtatatccatttgctttaccagaaggctaacttgttttagttctcattttcacctttgagacatttggaataaatatcaatgttaacataaattggaattttgactttgattttaggaccaatgaacaagccaagtacttaccctagtcatatataatccaactgtatggttatttggtattcattccacacttcattttacttgatctcccttaagattgcaagattgtgtttgcagtttttctgaaaatctggggctataaaagcatcaggacctcccccgtaggggaggtcgtgtgtttggggtccttacacaacaggttacccttgagcttcaggaaaagaactggctctcagttccccagttccagcttaatgggtctaattaggtcctgaccaaaaaggtggcagttcttttccctcatgtctcttcagcgctccccgagactctggagactctgtcatatccctagggctgagcctcccaggaaccattcggctgttgtggcatctgtgtatgccatgcccagtgctgaggacctagtaacaaacgacaaatgcacaggcacagtggcatttttgtggaactcgtattccagctgtgcgtctcagaagaagcgcacagctccctcctggctttcttaacatagtgagccacttccacttaagggtctccttacattccttgagtttaatcattcatggattcagaggaaagtcttttgatttttgcttttctttaaacagttcatttgaggtgacctaccccagtgactttgcaccaaccaccaagaaacttttttgcatgcttcccgcaccctgtgccaatcaagggaagggtttaaaggcctggcgtttttattcctcaaagaaaggttttgcacagtattttaaggttcaagtgcttctactttgtgttcagaagcaactgtcatatatactgtgaaatgacaccttttatttatccctttttatttatgcagtatgtccccttttattttggcagaattttttctaaatggtggtttaacattttcaagcacatttcattgtccaatattcatagtaaagaatgagagttaacaataaccagtcacattaaaacaagattcctgctgccagttgtgaaaccggttgtcttaggcgtggcagctgatgattgagactgtgatcaggaaaatttccactatttcatcaggcctaataggtagattgtgtctccaaatgaactgtgttgggtttccatgcttaaagcacaatagaggtggtgcaagaatctccatgagggcttaaatggcagtgatggttcaggcggtagagtttggagaagaagggatttgaaacaaaccaaaggaaagaaaagtaagtagccagaaatcacaaaatggcatttttctaaaaacaaaggaaaaggaataaaagaactaataagtttgaaacccctacccctcccaaatttggcagggggggaggtattttttttctatctatctaactaacccatctagaaaacagttgaccaaattatagacttctaaatgttaatctgctttctcagtttcagttgaaaagagactttgttttgcctactgcagaacttctaggttctttcttatagtcttggggttcttattatagatcgaaaatgtgagtcggcataattaagccattcggagtcttcagaagcagttcactcttgaaatgactccgtccgcctacagccatttaagatttcagaacaaaaacagatcttgattttctttttcatgttaactcaagctgttgctgagtgggagagtcagaaatgacaccagctccactgattactcagctgctgaaggatgattttttaaaatgcacctttactgtatatggacttcctaatttccacctgtagagcatcttagggaggctaacatgtcactctggatgttcttttagaataagatgcaaatctatttttctgaaggcattagagatagcaaacatttattgtgagtttactatatactaggcactgtgctaagtgttttgcatagaaagtttaaaattctggcttttttgttggcccaatcataagtttcatatcagttcaacattcaaattatattaaggtacttaagaagaatccctggctaaatgtgaggggcagtgccacagatggactgaaactttatgcttattgcacatttatgctattattatttgttgaattatagaaccaagggagtgtggaagccactggaaaaaatatgagacttagatacataatttgagtaaaaatggctcaaagtcatgagggtaaagttttttgtatttccattttattcgagcggcatcgtttttaaaaatcattatgaatttgaccctatatagatgtttccaaataattctttttcaccttcataaaattccttcctgtggctgtgagatgccttgcctatcagttttcaagcttagttgtctttctcatcctttaccattttagctttaaaaaacaaaagtgacaattagaacttcctgcctgctgggcctcactgaaagaccgatattggcctgataaggagatatttattttgttttagtggcttcagaaatccctctccctcagcaagctttccatcacggcccccccgtcagcatcttccctgatagcgttcttctctgtgtttattctggggcttcaggctcgcccaggaggaactgataaccgctggcaggagataacattctctaaggggctctcaaattggaatcgaatccctcaagccagtcagcctagagaatacatttaaagggttcagttctggagtttcacagagttcatttctagacctatcagatagcaagtgtggagttctttctcaactaaattcaagcagagacattttttagacgatgaaggatatttgcacaaaggcttcagcatgatcccccaaacctgctgcctctgaaggcatctccacacattgacagccaatgccttcagtgcgttcctagggcaggtgtcctggcttgagtgactgtcctccaataatcagagctcaaactaaacatcgtatgttttacttttggtttccaggcaaggctgagcagggaattttcagttttccctgcccagatgggtgttttttcctgaaggcatcatttattgtgtagcgaggagacagggctggctgtggcagggatagtctagaactgtcctcattgctgctgttcctaaatagtatctttaccaagtaataacgtgccgtctttgggaataagtgctttcctcttagcctgttctgttttcttgggtgcgctaagtaattgaactggctcaggaagtacctattgtggtttggcagaggtgactgtcacgccttgtgactccaggggccagcactgctgggatcctggctagaccagacagagccttggtgaagtgcttaggctgtctgcacatcgcgaggaaggtggtattcacttcgctaagctccttggcataggcagtttgaacagggctttatcaaattcgtattcaacaagagtagaagcgaaaattgatgactgtgtattacttgaaatgagtcttaatctttcacatttagttctcagggtatgctgatttcctttaggtaaaccatgaacatcagaaagacttttattaacctatgacagggtccccaccccagtatttttccactccattaaaatggaagttttttttttttttttcttttttgagacagagttttgctcttgttgcccagtctggagtgcaatggcacaatctcggctcaccacaacctccacctcccagattcaagcgattcttctgcctcagcctcccaagtagctgggattacaggtgtgcgccaccacgcccagctaattttgtatttttagtagagatggggtttctccatgttggtcaggctggtctcgaacttccgacctcaggtgatccgcccacctcggcctcccaaagtgctgggattacaggcaagagccactgcatccagcttaggctatcttactccagcctaaacagcaattttctatcataaggtctgtactaatgaaaacagaatcacccaaggctgctgtttgttctgtctgtgctgccattgtccgcattttgctgaggaggaaacggaactgcacttttgagtgagtggcccagagccttctagaatgagagtgcgttggaagccagatatgtggcgattgtgtcgccagctgttactcaggttttctcaagaaggaggagcaactttggcagttttgcttcagttctctctagccctctgtgtaatcgcccctttttctttatttcagcacaaacacagagcagtctaaagcaaccgagcactgagaaaaatgaactctgcccaaagaatgtcccaaagagagagtacagcgtgaaagaaatcctaaaattggactccaacccctccaaaggaaaggacctctaccgttctaacatttcacccctcacatcagaaaaggacctcgatgactttagaagacgtgggagccccgaaatgcccttctaccctcgggtcgtttaccccatccgggcccctctgccagaagactttttgaaagcttccctggcctacgggatcgagagacccacgtacatcactcgctcccccattccatcctccaccactccaagcccctctgcaagaagcagccccgaccaaagcctcaagagctccagccctcacagcagccctgggaatacggtgtcccctgtgggccccggctctcaagagcaccgggactcctacgcttacttgaacgcgtcctacggcacggaaggtttgggctcctaccctggctacgcacccctgccccacctcccgccagctttcatcccctcgtacaacgctcactaccccaagttcctcttgcccccctacggcatgaattgtaatggcctgagcgctgtgagcagcatgaatggcatcaacaactttggcctcttcccgaggctgtgccctgtctacagcaatctcctcggtgggggcagcctgccccaccccatgctcaaccccacttctctcccgagctcgctgccctcagatggagcccggaggttgctccagccggagcatcccagggaggtgcttgtcccggcgccccacagtgccttctcctttaccggggccgccgccagcatgaaggacaaggcctgtagccccacaagcgggtctcccacggcgggaacagccgccacggcagaacatgtggtgcagcccaaagctacctcagcagcgatggcagcccccagcagcgacgaagccatgaatctcattaaaaacaaaagaaacatgaccggctacaagacccttccctacccgctgaagaagcagaacggcaagatcaagtacgaatgcaacgtttgcgccaagactttcggccagctctccaatctgaaggtaggccttgagagagagcagtccaaggggctgtgagtgcatgcttgtgtttgtatttagcttgctttccatggggtatcgattgcatttgcagtagtatgagcccccggttggggatagtgggtatggattccgcctggcttttgccacttctagctctttgactttggacaagtgacttcccttctcctgattttcttctgaataataaaaaaattaggggtttggactagaagattaggtgaaactccctgctagcctgtgatttttgtgcttttaagaaaaacaccattctgaaaacatgaagatttcttctttttaagactgtcttgatgcttttcttaagatatttgcatcaacacttgagtcttggagcagaaatgttaggtctcagagccagcttgagagcagagctaacacatgtggcttcttcccaggtccacctgagagtgcacagtggagaacggcctttcaaatgtcagacttgcaacaagggctttactcagctcgcccacctgcagaaacactacctggtacacacgggagaaaagccacatgaatgccaggtgcgcagtattttctgggtagaccttctgacctttgtagaaaatgtctgtgagtcaccctcccatgtcctatatagcccgtagttaaagccaacaccagattctgcgttgtcccatcctggactgatggcactatggtccttcccagtactttgtatctgctgatgacttgagatggcacagccagcttccagtgggtgggaaaatggtaggggaaataaacagcccctcgtgtgctgtgtgcccacatccccccgtttgcttaataccacactggaggtgccacaaggaggcttctcacctcctaggttgctgggcgttggccggtaagcctgcccctcccgttggcaactcttaatcttctggccttcctgtctcccttccctgctgtctctctcccctacactgtaggtctgccacaagagatttagcagcaccagcaatctcaagacccacctgcgactccattctggagagaaaccataccaatgcaaggtgtgccctgccaagttcacccagtttgtgcacctgaaactgcacaagcgtctgcacacccgggagcggccccacaagtgctcccagtgccacaagaactacatccatctctgtagcctcaaggttcacctgaaagggaactgcgctgcggccccggcgcctgggctgcccttggaagatctgacccgaatcaatgaagaaatcgagaagtttgacatcagtgacaatgctgaccggctcgaggacgtggaggatgacatcagtgtgatctctgtagtggagaaggaaattctggccgtggtcagaaaagagaaagaagaaactggcctgaaagtgtctttgcaaagaaacatggggaatggactcctctcctcagggtgcagcctttatgagtcatcagatctacccctcatgaagttgcctcccagcaacccactacctctggtacctgtaaaggtcaaacaagaaacagttgaaccaatggatccttaagattttcagaaaacacttattttgtttcttaagttatgacttggtgagtcagggtgcctgtaggaagtggcttgtacataatcccagctctgcaaagctctctcgacagcaaatggtttcccctcacctctggaattaaagaaggaactccaaagttactgaaatctcagggcatgaacaaggcaaaggccatatatatatatatatatatatctgtatacatattatatatacttatttacacctgtgtctatatatttgcccctgtgtattttgaatatttgtgtggacatgtttgcatagccttcccattactaagactattacctagtcataattattttttcaatgataatccttcataatttattatacaatttatcattcagaaagcaataattaaaaaagtttacaatgactggaaagattccttgtaatttgagtataaatgtatttttgtcttgtggccattctttgtagataatttctgcacatctgtataagtacctaagatttagttaaacaaatatatgacttcagtcaacctctctctctaataatggtttgaaaatgaggtttgggtaattgccaatgttggacagttgatgtgttcattcctgggatcctatcatttgaacagcattgtacataacttgggggtatgtgtgcaggattacccaagaataacttaagtagaagaaacaagaaagggaatcttgtatatttttgttgatagttcatgtttttcccccagccacaattttaccggaagggtgacaggaaggctttaccaacctgtctctccctccaaaagagcagaatcctcccaccgccctgccctccccaccgagtcctgtggccattcagagcggccacatgacttttgcatccattgtattatcagaaaatgtgaagaagaaaaaaatgccatgttttaaaaccactgcgaaaatttccccaaagcataggtggctttgtgtgtgtgcgatttgggggcttgagtctgggtggtgttttgttgttggtttttgttgctttttttttttttttttttttaatgtcaaaattgcacaaacatggtgctctaccaggaaggattcgaggtagataggctcaggccacactttaaaaacaaacacacaaacaacaaaaaacgggtattctagtcatcttggggtaaaagcgggtaatgaacattcctatccccaacacatcaattgtattttttctgtaaaactcagattttcctcagtatttgtgtttttacattttatggttaatttaatggaagatgaaagggcattgcaaagttgttcaacaacagttacctcattgagtgtgtccagtagtgcaggaaatgatgtcttatctaatgatttgcttctctagaggagaaaccgagtaaatgtgctccagcaagatagactttgtgttattctatcttttattctgctaagcccaaagattacatgttggtgttcaaagtgtagcaaaaaatgatgtatatttataaatctatttataccactatatcatatgtatatatatttataaccacttaaattgtgagccaagccatgtaaaagatctactttttctaagggcaaaaaaaaaaaaaaaaaaaaaagaacactcctttctgagactttgcttaatacttggtgacctcacaatcacgtcggtatgattgggcacccttgcctactgtaagagaccctaaaaccttggtgcagtggtggggaccacaaaacaaccagggaggaagagatacatcattttttagtattaaggaccatctaagacagctctattttttttttgccactttatgattatgtggtcacacccaagtcacagaaataaaaaactgactttaccgctgcaatttttctgttttcctccttactaaatactgatacattactccaatctattttataattatatttgacattttgttcacatcaactaatgttcacctgtagaagagaacaaatttcgaataatccagggaaacccaagagccttactggtcttctgtaacttccaagactgacagctttttatgtatcagtgtttgataaacacagtccttaactgaaggtaaaccaaagcatcacgttgacattagaccaaatacttttgattcccaactactcgtttgttctttttctccttttgtgctttcccatagtgagaatttttataaagacttcttgcttctctcaccatccatccttctcttttctgcctcttacatgtgaatgttgagcccacaatcaacagtggttttattttttcctctactcaaagttaaaactgaccaaagttactggctttttactttgctagaacaacaaactatcttatgtttacatactggtttacaatgttatttatgtgcaaattgtcaaaatgtaaattaaatataaatgttcatgctttacc

(19) United States
(12) Patent Application Publication
(10) Pub. No.: US 2008/0060087 A1
(43) Pub. Date: Mar. 6, 2008

(54) Modified Cells and Methods of Using Same

(75) Inventors: Axel Kallies, St Kilda (AU); Jhagvaral Hasbold, North Balwyn (AU); David Tarlington, Blackburn (AU); Lynn Corcoran, East Kew (AU); Philip Desmond Hodgkin, Coburg (AU); Stephen Laurence Nutt, Ascot Vale (AU)

Correspondence Address: SCULLY SCOTT MURPHY & PRESSER, PC, 400 GARDEN CITY PLAZA, SUITE 300, GARDEN CITY, NY, 11530, US

(73) Assignee: Davies Collison Cave, Melbourne (AU)

(21) Appl. No.: 10/589,321

(22) PCT Filed: Feb. 11, 2005
(86) PCT No.: PCT/AU05/00168

§ 371 Date: Nov. 22, 2006

(30) Foreign Application Priority Data
Feb. 12, 2004 (AU) 2004900673

(51) Int. Cl. A01K 67/00; C12N 15/00; C12N 5/06; C12Q 1/68

(52) U.S. Cl. 800/3; 435/25; 435/320.1; 435/349; 435/354; 435/6; 800/13; 800/14; 800/16; 800/17; 800/19

(57) ABSTRACT

In one aspect, the present invention provides a genetically modified cell or non-human organism comprising such cells comprising modified genetic material which when expressed produces a polypeptide co-expressed with a reporter molecule and wherein the polypeptide is associated with terminal differentiation of a haematopoietic cell. Preferably, the genetic material gene is a Blimp allele or a part, fragment or functional form thereof. Furthermore, the identification of the reporter molecule in B-cell lineage cells indicates that such cells are committed to differentiate or have differentiated into ASC. Alternatively, reporter molecule activity in cells of a T cell lineage indicates that these cells are activated. Thus, as described herein, the presence of Blimp in a lymphocyte indicates that the cell is terminally differentiated or is committed to terminal differentiation. Exemplary T-cells include CD4⁺ T-cells and CD8⁺ T-cells and exemplary B-cells are ASC.

### BACKGROUND OF THE INVENTION

1. Field of the Invention

The present invention relates generally to a model system to identify haematopoietic cells of particular lineages and their stage of differentiation. More particularly, the present invention provides genetically modified cells and non-human animals comprising such cells which carry a genetic marker of terminal differentiation modified to co-produce a reporter molecule capable of eliciting an identifiable signal and their use in identifying molecules capable of modulating the differentiation or transformation status of cells, such as, without limitation, embryonic cells during development, cells with aberrant differentiation such as cancer cells, and cells of the haematopoietic cell lineages such as, for example, B and/or T cells. Identified molecules form the basis for pharmaceutical compositions for therapeutic and prophylactic application.

2. Description of the Prior Art

Bibliographic details of references in the subject specification are also listed at the end of the specification.

Reference to any prior art in this specification is not, and should not be taken as, an acknowledgement or any form of suggestion that this prior art forms part of the common general knowledge in any country.

Cellular life involves a myriad of alternative and highly regulated biochemical pathways directing changes in cell division, differentiation, morphogenesis and apoptosis. Cells vary in their potential to divide and/or differentiate. For example, the embryo comprises totipotent cells retaining the ability to differentiate into any cell type. Other cell types including stem cells are pluripotent and may ultimately differentiate into a range of but not all cell phenotypes. Some cells become committed to one final form: they are terminally differentiated.

Changes which block normal maturation of cells into terminally differentiated cells or which prevent apoptosis can act as triggers for tumor development characterized by uncontrolled cell division without differentiation or cell death. Thus, agents which promote differentiation and normal apoptosis may switch off tumor development.

Molecules which are expressed during the time of terminal differentiation of particular cell types have been intensely studied. However, in order to understand the sequence of events during this period at a molecular level it is necessary to understand the temporal and spatial expression patterns of molecules which are expressed in this phase of development.

B-lymphocyte-induced maturation factor (Blimp) is a 98 kDa transcription factor which was originally identified as being induced during the differentiation of a B-cell lymphoma cell line (Turner et al., Cell 77:297, 1994). The corresponding factor from human cells is referred to as PRDM-1. It has been proposed that Blimp-1 has a pre-eminent role in regulating B-cell terminal differentiation. Specifically, Blimp-1 is expressed in antibody secreting cells (ASC) from man and mouse but it is not expressed in memory cells (Angelin-Duclos et al., J Immunol 165:5462, 2000). Ectopic expression of Blimp-1 is sufficient to drive terminal differentiation of lymphomas and primary B-cells into ASC cells (Turner et al., (supra), Schliephake et al., Eur J Immunol 26:268, 1996; Messika et al., J Exp Med 188:515, 1998; Knodel et al., Eur J Immunol 31:1972, 2001). Blocking expression of Blimp-1 through antisense or dominant-interfering approaches suppresses cell-cycle exit which is thought to be essential for full ASC differentiation (Soro et al., J Immunol 163:611, 1999; Angelin-Duclos et al., J Immunol 165:5462, 2000; Johnson et al., Eur J Immunol 32:3765, 2002). Also, mice which lack Blimp-1 in B-cells produce very little immunoglobulin and have a markedly reduced ASC compartment. (Shapiro-Shelef et al., Immunity 19:607, 2003).

It was initially reported that Blimp-1 is only produced in cells of the B-cell lineage, however, it is now evident that Blimp-1 is also produced during myeloid differentiation (Keller et al., Genes Dev 5:868, 1991, Chang et al., Nat Immunol 1:169, 2000). Blimp-1 is required for the repression of c-myc which is involved in myeloid differentiation (Chang et al., (supra), 2000; Marcu et al., Annu Rev Biochem 61:809, 1992). Over production of Blimp-1 in U937 cells for example is sufficient to induce macrophage differentiation (Chang et al., (supra), 2000). Thus, repression of c-myc by Blimp-1 in macrophages and B-cells is a feature of terminal differentiation in these two lineages. Blimp-1 is also broadly produced during mouse and Xenopus embryonic development (de Souza et al., Embo J 18:6062, 1999; Rosenbaum et al., Embo J. 9:897, 1990).

B-lymphocytes are among the most intensively studied eukaryotic cell types but while the early steps of B-cell development are relatively well characterized, much less is known about the processes which control the final differentiation of B-lymphocytes into ASC. ASC (plasma cells) are the direct mediators of the humoral immune response. They secrete a large amount of serum immunoglobulin essential for protective immunity. The terminal differentiation of B-lymphocytes into ASC is, therefore, a subject of intense therapeutic interest. For example, terminal differentiation to ASC is a crucial element in effective vaccination strategies. Furthermore, multiple myeloma results from the failure of an ASC to complete the differentiation pathway.

However, ASC represent a very rare population of highly specialised cells located mostly in the bone marrow and spleen. ASC populations in mice and man comprise cells of heterogeneous life span and cell surface phenotype making a definitive prospective isolation of pure ASC impossible (Fong et al., Proc Natl Acad Sci U S A 11:11, 2003; Medina et al., Blood 99:2154, 2002; O'Connor et al., J. Exp Med 195:737, 2002; Manz et al., Curr Opin Immunol 14:517, 2002; Underhill et al., Blood 24:24, 2003).

T-cell terminal differentiation programs are poorly understood (Sprent et al., Immunol Lett 85:145-149, 2003). In response to infection, antigen-specific T cells differentiate into effector cells and undergo massive clonal expansion. Homeostasis of T cell numbers is maintained by the subsequent contraction phase where >90% of effector cells are eliminated with a small fraction becoming memory T cells (Sprent et al., Annu Rev Immunol 20:551-579, 2002). This process has been proposed to be under genetic control as the contraction is independent of the dose or duration of infection (Badovinac et al,, Nat Immunol 5:809-817, 2004; Badovinac et al., Nat Immunol 3:619-626, 2002). The ability to control T cell numbers is essential as enhanced expansion due to the lack of T-regulatory cells (Khattri et al., Nat Immunol 4:337-342, 2003; Hori et al., Science 299:1057-1061, 2003; Fontenot et al,, Nat Immunol 4:330-336, 2003), the loss of the down-regulatory molecule CTLA-4 (Chambers et al., Immunity 7:885-895, 1997) or genetic deficiencies in non-obese diabetic (NOD) mice result in autoimmunity.

The ability to monitor terminal differentiation of ASC, T-cells and other cells of the haematopoietic system in a wide range of contexts and under various stimuli would be extremely valuable in developing strategies and reagents for use in the treatment and/or prophylaxis of a range of conditions associated with aberrant differentiation, such cancer autoimmune disease, or with harnessing normal developmental programs such as in the development of an appropriate immune response.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of sequence identifiers is provided in Table 1. A sequence listing is provided after the claims.

Genes and other genetic material (eg mRNA, constructs etc) are represented in italics and their proteinaceous expression products are represented in non-italicised form. Thus, the transcription factor Blimp is the expression product of Blimp. The term “Blimp” or “Blimp” is used to denote all homologs or variant molecules derived from any animal or mammalian species, including a human homolog. Accordingly, human PRDM-1 and its product, PRDM-1 are encompassed in the terms Blimp or Blimp. Unless otherwise stated, reference to Blimp is a reference to a functional form of the polypeptide and reference to a modified Blimp is a reference to the gene or allele sequences encoding a functional form of Blimp.

The present invention is predicated, in part, on the identification of the role of Blimp in the differentiation of haematopoietic and embryonic cells. By screening for the presence of Blimp, or the level of Blimp, a determination can be made as to the stage of terminal differentiation of a cell. The identification of the role of Blimp further enables substantially homogeneous populations of particular haematopoietic cells to be identified such as, but not limited to, ASC (plasma cells).

More particularly, the present invention provides a genetically modified cell or an in vivo or in vitro system comprising cells which co-express genetic material which encodes Blimp and a reporter molecule. Detection of reporter activity in cells of a haematopoietic lineage, such as but not limited to a lymphocyte lineage, is indicative that cells having reporter activity and producing functional Blimp are committed to terminal differentiation. Thus, the detection of reporter-active B-cells producing functional Blimp is an indication that these cells are committed to differentiate into an antibody secreting cells (ASC). Also, as described herein the detection of reporter activity in T cells expressing a functional Blimp, is indicative that these cells are activated/memory T cells, such as activated CD4⁺ T-cells or effector CD8⁺ T-cells. The present invention provides therefore, genetically modified cells or non-human animals comprising such cells which facilitate monitoring the differentiation or transformation status of particular cells under various conditions or in the presence of various stimuli or agents. The present invention further provides screening methods, including high through-put screening methods, for identifying molecules capable of modulating the differentiation or transformation status of cells, such as, without limitation, embryonic cells including stem cells during development, cells with aberrant differentiation such as cancer cells, and cells of the haematopoietic cell lineages such as, for example B and/or T cells.

Specifically, a genetically modified cell, or a non-human organism comprising such cells, is provided by the present invention. In one embodiment, the cells produce Blimp translated from an mRNA modified to encode a reporter molecule. Preferably, the reporter molecule encoding sequence is inserted into an intron of a Blimp allele. When the modified Blimp allele is present in heterozygous form, the other allele will express a functional Blimp. In some embodiments, the modified allele may express a functional Blimp polypeptide or a functional form thereof. In other embodiment the modified allele expressed a non-functional Blimp polypeptide. In one embodiment the modified cells are useful in in vivo or in vitro cellular model systems to identify and isolate, inter alia, ASC. In another embodiment, the modified cells are useful for monitoring the differentiation status of haematopoietic such as T-cells and/or B-cells in a wide range of assays.

In one aspect, the present invention provides a genetically modified cell or non-human organism comprising such cells comprising modified genetic material which when expressed produces a polypeptide co-expressed with a reporter molecule and wherein the polypeptide is associated with terminal differentiation of a haematopoietic cell. Preferably, the genetic material is a Blimp gene or a part, fragment, homolog, derivative or functional form thereof. Furthermore, the identification of the reporter molecule in B-cell lineage cells indicates that such cells are committed to differentiate or have differentiated into ASC. Alternatively, reporter molecule activity in cells of a T cell lineage indicates that these cells are activated. Thus, as described herein, the presence of Blimp in a lymphocyte indicates that the cell is terminally differentiated or is committed to terminal differentiation. Exemplary T-cells include CD4⁺ T-cells and CD8⁺ T-cells and exemplary B-cells are ASC. Where a non-functional Blimp polypeptide is produced, detection of reporter-active cells indicates that the cells have been exposed to conditions sufficient to render them terminally differentiated if they had been able to produce a functional Blimp polypeptide.

Genetically modified non-human organisms may be provided in the form of gametes, embryos or ES cells for transplantation. Embryos are preferably maintained in a frozen state and may optionally be sold with instructions for use. Targeting constructs and genetically modified cells are also preferably maintained in a frozen state and may optionally be sold with instructions for use. All such cells are referred to herein as an in vivo or in vitro cellular model system.

The present invention provides a system for monitoring gene expression and differentiation fate in cells in vivo and in vitro at the single cell, tissue and organism level. Thus, reporter activity may be monitored in live cells and gene expression monitored in fixed tissues. Preferably, the reporter expression cassette encodes a fluorescent or other light emitting moiety. The availability of organisms and cells which report the expression of Blimp-1 for example as a marker for terminal differentiation of a particular lineage or cell will be an extremely useful tool in a wide range of applications. In relation to cells of the B-cell lineage, this system finds broad application in the study, isolation and monitoring of ASC. As previously mentioned, ASC have not hitherto been available for study although these cells are crucial for an effective antibody response. Furthermore, aberrant differentiation in ASC causes multiple myeloma in man making them an important cell type to study for this reason.

In a related embodiment, the present invention provides a method for phenotyping and/or monitoring a cell of the haematopoietic system comprising screening a genetically modified cell or non-human animal comprising such cells comprising a modified Blimp gene encoding a Blimp protein which when expressed co-expresses Blimp or a part, fragment, variant, homolog or functional or non-functional form thereof and a reporter molecule, wherein detection of reporter activity is indicative or predictive of a cellular phenotype and/or commitment of a cell to terminally differentiate. Haematopoietic cells include without limitation B-cells, T-cells, dendritic cells, macrophages, natural killer cells, granulocytes, erythrocytes, eosinophils, megakaryocytes, bone marrow, splenic, dermal, or stromal cells or their derivatives. In one particular embodiment, the haematopoietic cells are lymphocytes such as B and/or T cells.

In a further embodiment, cells which exhibit reporter activity or changes in reporter activity are isolated or selected from among cells which do not exhibit reporter activity. Isolation of reporter-active cells may be by any convenient method. For example, flow cytometry, laser scanning cytometry, chromatography and/or other equivalent procedures are conveniently employed. Flow cytometric procedures are particularly preferred. Additionally, further selection markers such as for example drug selection markers, may be used to isolate or select the modified cells of the present invention.

The present invention also provides antagonists and agonists of Blimp-1 expression or Blimp-1 activity. One example of an agonist of Blimp-1 expression is a cytokine such as but not limited to IL-21. Pharmaceutical compositions are further contemplated comprising recombinant, synthetic or isolated forms of the present agonists and antagonists and one or more pharmaceutically acceptable carriers, diluents or excipients. Reference to Blimp-1 expression or production of Blimp-1 protein includes in a single cell or within a population of cells.

TABLE 1
Summary of sequence identifiers

| SEQUENCE ID | |
|---|---|
| NO: | DESCRIPTION |
| 1 | Nucleotide sequence encoding murine Blimp-1 |
| 2 | Amino acid sequence of murine Blimp-1 |
| 3 | Nucleotide sequence encoding human Blimp-1 |
| | (PRDM-1) |
| 4 | Amino acid sequence of human Blimp-1 (PRDM-1) |
| 5 | Genomic nucleotide sequence of murine Blimp-1 |
| 6 | Genomic nucleotide sequence of human Blimp-1 |
| | (PRDM-1) |

### BRIEF DESCRIPTION OF THE FIGURES

FIG. 1 is a diagrammatic representation of the Blimp-1 locus and a targeting strategy. A) Structural domains of the Blimp-1 protein. The segment of the protein encoded by exons 7-8 are indicated. Acidic, N and C terminal acidic regions; PR, region of homology to the retinoblastoma interacting zinc finger protein RIZ; Pro, proline rich region; Zn, 5 Zinc fingers. B) Genomic locus of Blimp-1, indicating the 8 exons as boxes and introns as black lines. Coding regions are in grey, non-translated regions are white. Restriction enzymes used for Southern hybridisations are marked, along with the 5′ and 3′ probes. Targeted allele derived from the homologous recombination event and subsequent manipulations is indicated C) Southern hybridisation on targeted and control ES cell DNA, using 5′ and 3′ ends of the Blimp-1 locus, to show expected products of the targeting event (4.8 kb 5′ arm and 4.5 kb 3′ arm). Expression of Blimp-1 in blimp^{gfp/+} LPS stimulated B cells cultured for 0-3 days ex vivo in IL-15 ^{+/-}IL-21. Blimp-1 expression was detected using a monoclonal antibody against mouse Blimp-1, a goat polyclonal antibody against α-actin was used as a loading control. ^{+/+}, wild type C57B1/6 mice; ^{-/T}blimp^{gfp/+} mice.

FIG. 2 is a graphical representation showing the results of FACS analysis of Blimp^{gfp} expression in B-cells in vivo. A) Syndecan-1 and Blimp^{gfp} expression in lymph nodes, spleen and bone marrow in Blimp^{gfp/+} mice (upper panel) and controls (lower panel). B) Expression of Blimp^{gfp} in B220 positive B cells.

FIG. 3 is a graphical and photographic representation showing the results of ELIspot analysis of Blimp^{gfp} sorted cells. Gfp positive cells were sorted from bone marrow (BM) and spleen of an untreated Blimp^{gfp/+} mouse and analysed in an EliIspot assay. Isotype specific antibodies or anti kappa antibodies were used to coat the elispot plate and to detect secreted immunglobulins. A) Distribution of isotype specific immunglobulins in 200 gfp-positive sorted cells (one representative experiment of three). B) Detection of kappa chain in a single representative well of an ELIspot plate (sample: sorted bone marrow cells). left, input 200 gfp-positive cells; middle, input 100 000 gfp-negative cells; right, input 100 000 unsorted cells.

FIG. 4 is a graphical representation of the results of FACS analysis showing induction of antibody secreting cells with LPS in vivo. Blimp^{gfp/+} mice were i.v. injected with 2 ug E. coli LPS. Spleens A) and bone marrows B) of these mice were analysed at indicated time points after LPS treatment. LPS induces the formation of ASC, increasing the frequency from about 0.5% to about 5% at day 3 in spleen and from about 0.05% to about 0.25% at day 4 in the bone marrow, respectively, upper panel, FACS scans for syndecan-1 and Blimp^{gfp}, middle panel, syndecan-1 and B220 in gfp-positive gated cells, lower panel, histograms for syndecan-1 and B220 expression in GFP-positive cells at indicated time points.

FIG. 5A is a graphical representation of the kinetics of Blimp^{gfp} expression. Flow cytometry histograms of Blimp^{gfp} expression by stimulated B cells from Blimp^{gfp/+} mice (red line) and wild type C 57B1/6 mice (blue me) are shown. Histogram gates show a percentage of Blimp^{gfp} positive populations. Highly purified small resting B cells were stimulated recombinant CD40L, IL-4 and IL-5 (top panels) or LPS (20 ug/ml) (bottom panels). Cells were harvested different days of culture time and analysed on flow cytometry. LPS stimulated cells start to express Blimp at 2 days, while in response to CD40L and IL-4/IL-5 Blimp expression become evident 3 days.

FIG. 5B is a graphical representation showing that Blimp^{gfp} positive cells secrete antibodies. Blimp^{gfp/+} B cells were stimulated with LPS for four days. Cells were harvested and stained with Syndecan-1 (Synd-1) specific antibodies and GFP expressing (left panel, A-C) and non-expressing regions (left panel, D) were sorted directly to the Elispot plates coated with various isotype specific antibodies, using automated cell deposition unit. Sorted cells were processed according to the standart Elispot method. Right panels show number of Ig secreting cells in sorted regions. Most Blimp^{gfp} cells secrete Ig, while all Blimp^{gfp} negative cells do not secrete any of Ig isotypes tested.

FIG. 5C is a graphical representation showing the different expression of Blimp^{gfp} in response to various stimuli. Highly purified small resting B cells were stimulated with i) recombinant CD40L and IL-4; ii) CD40L, IL-4 and IL-5; iii) LPS; iv) LPS and IL-4; v) LPS and anti-IgD monoclonal antibody. After four days of culture cells were harvested, stained with Synd-1 specific antibody and analysed on flow cytometry. Shown here are two parameter dot plots of flow cytometry analysis.

FIG. 6 is a graphical representation showing the results of analyses of mice transplanted with activated B-cells. Purified resting splenic B-cells of Blimp^{gfp/+} mice were activated for three days in the presence of 20 ug/ml LPS. 3×10⁶ cells (containing about 2×10⁶ gfp positive cells, i.e. antibody secreting cells, A) were washed three times with LPS and transplanted into WT recipients by i.v. injection. After 7 days the recipient mice were analysed for the presence of donor ASC (B).

FIG. 7A is a tabulated summary of genotyping results of mice born from Blimp^{gfp/+}×blimp^{gfp/+} matings. FIG. 7B is a photographical representation of representative PCR results of genotyping of mice weaned (left) or embryos at day E9.5 (right).

FIG. 8 is a photographic and graphical representation of splenocytes of Blimp^{gfp/gft} and Blimp^{gfp/+} reconstituted mice were cultured in the presence of 20 ug/ml LPS and analysed for the presence of GFP positive, i.e. antibody secreting cells, at day three (A). GFP positive cells of both cultures were than sorted (B, gate R1) and analysed in an ELIspot assay. While Blimp^{gfp/+} cells yielded 60-70% antibody secreting cells (B, lower panel left), Blimp^{gfp/gft} gave only 5-7% antibody secreting cells which produced only tiny ELIspot's (B, lower panel, right) compared to spots produced by heterozygous cells. Detection of IgM and kappa chain in single representative wells of an ELISPOT plate (input 200 gfp-positive cells).

FIG. 9 is a graphical representation of the results of FACS analysis of bone marrow derived macrophages (BMM) and blood monocytes. Bone marrow cells were cultured for 7 days in the presence of 10 ng/ml rMCSF, medium was changed and non-adherent cells were removed at day 3 and 5 of culture. Adherent cells (BMM) were analysed for Blimp^{gfp} expression (left panel). Further, MacI/Gr1 double positive blood cells were analysed in FACS (right panel) (black line—wildtype, red line—Blimp^{gfp/+}).

FIG. 10 is a graphical representation showing FACS analysis in vitro generated dendritic cells (DC's). Bone marrow cells were cultured for 8 days in the presence of 100 ng/ml Flt3 ligand. Cells were than cultured for another 24 hours (left column) or were stimulated with CpG (1.5 uM), GMCSF (50 ng/ml), gIFN (20 ng/ml) and IL-4 (20 ng/ml) (middle column) or with 1 ug/ml LPS (right column). Blimp^{gfp} expression is shown in a histograms for plasmacytoid DC's and conventional DC's (solid line—wildtype, dotted line—Blimp^{gfp/+}).

FIG. 11 is a graphical representation showing FACS analysis of T cells in vivo and in vitro. Thymic (left) and lymph node (middle) T cells, and in vitro activated CD4⁺/CD8⁺ purified lymph node cells (right) of Blimp^{gfp/+} mice were analysed in FACS. Blimp^{gfp} expression levels of gated T cell populations are shown in histograms (lower panel; black line—wildtype, red line—Blimp^{gfp/+}).

FIG. 12 is a graphical representation showing Blimp-1 expression in the NK lineage can be detected in the Blimp^{gfp/+} reporter mice and induced by maturation stimuli. A) in vivo splenic NK cells are GFP⁺. B) Sorted NK cells from Blimp^{gfp/+} spleens were cultured for 4 days in IL-15, followed by 2 days in the indicated cytokine. mfi, mean fluorescence index of Blimp^{gfp}. C) Expression of Blimp-1 in ^{+/+}NK cells cultured for 7 days ex vivo in IL-15 ^{+/-}IL-21. Blimp-1 expression was detected using a monoclonal antibody against mouse Blimp-1, a goat polyclonal antibody against α-actin was used as a loading control.

FIG. 13 is a representation showing the cDNA and predicted amino acid sequence of mouse Blimp-1/PRDM-1. The coding sequence is shown in upper case.

FIG. 14 is a representation showing the amino acid sequence of mouse Blimp-1/PRDM-1 derived from the nucleotide sequence (upper case) in FIG. 13.

FIG. 15 is a representation showing the cDNA and predicted amino acid sequence of human Blimp-1/PRDM-1. The coding sequence is shown in upper case.

FIG. 16 is a representation showing the amino acid sequence of human Blimp-1/PRDM-1 derived from the nucleotide sequence (upper case) in FIG. 15.

FIG. 17 is a representation showing the genomic nucleotide sequence of mouse Blimp-1. The genomic locus comprises 8 exons in bold upper case. ATG and stop codons are underlined.

FIG. 18 is a representation showing the genomic nucleotide sequence of human Blimp-1. The genomic locus comprises 8 exons in upper case, bold. ATG and stop codons are underlined.

FIG. 19 is a graphical representation showing that Blimp-1 is expressed in activated/memory T cells, Blimp-1 deficient T cells have an activated/memory phenotype. A) Gated CD4⁺ or CD8⁺ splenic T cells of the indicated genotype were examined for GFP fluorescence and activation state. The majority of CD62L^{low} CD4⁺ T cells and CD44^{high} CD8⁺ T cells from Blimp^{gfp/+} mice is low for GFP, only a small number of CD4⁺ T cells (CD62L low or high) is GFP high Blimp GFP is strongly expressed in the same population in Blimp^{gfp/gfp} T cells (dot blots); phenotype of Blimp-1 deficient CD⁴⁺ and CD8⁺ splenic T cells (histograms, solid line blimp-1^{+/+}, dotted line blimp-1 gfp/gfp). B) in vitro culture induces Blimp-1 expression. Naïve CD40⁺ T cells were grown for two rounds in Th1/Th2 polarizing conditions. C) Western blotting for wild type Blimp-1 protein in CD4⁺ cells grown as above in Th1 or Th2 conditions. Anti-Zap-70 is used as a loading control. B cells stimulated for four days with LPS to induce plasma cell differentiation were used as a positive control. D) Mice reconstituted with the indicated genotypes were analysed post- HSV infection for the appearance of CD8⁺ T cells specific for the dominant gB₄₉₈₋₅₀₅ epitope using a gB specific tetramer. E) in vitro cultured gB-specific CTL show normal cytotoxic function. The HSV infection data is representative of at least three mice of each genotype.

FIG. 20 is a representation of the molecular analysis of Blimp-1 positive CD40⁺ T cell populations. A) Blimp-1 is expressed in CD25⁺ suppressor T cells. CD25⁺ and CD25-CD40⁺ T cells were sorted (left panel), and naïve CD4⁺ were differentiated under Th1 and Th2 polarizing conditions (right panel), all populations were subjected to RT-PCR analysis. B) Blimp-1 deficient CD4⁺ cells secrete high amounts of IFNg and show defective IL-10 secretion. CD⁴⁺ cells were either sorted ex vivo from the spleen and restimulated with plate bound anti CD3 and CD28 for 24 h or differentiated into Th1 or Th2 cells in vitro and subjected to re-stimulation. IL-10 and IFNg in the supernatant was detected in an ELISA.

FIG. 21 is a representation showing that Blimp-1 deficient mice develop a lethal lymphocyte hyperproliferative syndrome. A) Histological examination of Rag1^{-/-} mice reconstituted with control or Blimp^{gfp/gfp} foetal liver derived stem cells. Blimp^{gfp/gfp} reconstituted mice were sacrificed when moribund. The normal histological appearance of the organs from Blimp^{gfp/+} mice is contrasted with the lymphocyte infiltration observed in Blimp^{gfp/gfp} mice. B) rapid onset of morbidity in Blimp^{gfp/gfp} but not Blimp^{gfp/+} reconstituted mice. The number of animals of each genotype examined is indicated.

FIG. 22 is a graphical representation of data showing that Blimp-1 regulates homeostatic proliferation of T cells. A) 3×10⁶ Naïve CD4⁺ or CD8⁺ splenic T cells from the indicated genotypes were adoptively transferred into non-irradiated Rag2^{-/-} recipients. Recipients were monitored for weight loss and signs of distress. Graph indicates the percentage changes in weight over the 3-4 week period. 4-6 mice were reconstituted with cells of each genotype. B) Mice that lost >10% body weight were sacrificed and splenic T cell numbers determined. C) splenomegaly of representative Rag2^{-/-} mice after transfer of Blimp^{gfp/gfp} CD8⁺ T cells. D) Flow cytometric analysis of Blimp^{gfp} expression in donor T cells 3 weeks after transfer.

FIG. 23 is a representation of data showing that Blimp-1 is induced after secondary stimulation and regulates cytokine responsiveness. A) Naïve CD8⁺ T cells of the indicated genotypes were cultured in the presence of anti-CD3/CD28 and IL-2 for 5 days before being re-seeded into secondary cultures containing IL-21 for 5 days. B) Identical cell cultures to above were stimulated with anti-CD3/CD28 with or without the indicated cytokine combinations. Total live cell number was determined at the indicated time-points in primary or secondary cultures. CD44^{high} activated/memory CD8⁺ cells were subjected only to primary culture.

### DETAILED DESCRIPTION OF THE PREFERRED EMBODIMENTS

The present invention is predicated, in part, by the development of a method for identifying and isolating cells of the haematopoietic system or embryonic cells and/or monitoring the differentiation of haematopoietic or embryonic cells, the method comprising detecting or quantifying the presence of a polypeptide (via a reporter) whose presence is associated with terminal differentiation of the cells.

In a particularly preferred embodiment, the polypeptide is Blimp or a part, fragment or functional form thereof which is co-expressed with a reporter molecule.

Accordingly, one aspect of the present invention provides a genetically modified cell or non-human organism comprising such cells comprising genetic material encoding a polypeptide which when expressed produces the polypeptide co-expressed with a reporter molecule and which polypeptide is associated with a cellular phenotype including a commitment in the cell to terminally differentiate.

In a further aspect, the present invention provides a genetically modified cell or non-human organism comprising such cells comprising a modified Blimp gene encoding a Blimp polypeptide which when expressed produces Blimp or a part, fragment or functional form thereof co-expressed with a reporter molecule and wherein the presence of Blimp is associated with a cellular phenotype including a commitment in the cell to terminally differentiate.

In a further preferred aspect, the present invention provides a genetically modified cell or non-human organism comprising such cells comprising a modified Blimp gene encoding a Blimp mRNA transcript comprising a Blimp coding sequence or a part, fragment or functional form thereof and a reporter molecule encoding sequence, wherein the presence of Blimp is associated with a cellular phenotype including a commitment in the cell to terminally differentiate.

Preferably, the reporter molecule encoding sequence is inserted with an intron of the Blimp allele. In this way, the modified Blimp allele co-produces the reporter from a bicistronic RNA under the control of endogenous Blimp regulatory elements.

The terms “co-expression” and “co-production” are used herein in a broad sense to refer to the transcription of two or more nucleic acid regions (expressed as one or more RNAs) at the same time or at substantially the same time and their subsequent translation (produced as one or more polypeptides) at the same or substantially the same time. Preferably, one transcript is expressed which encodes both Blimp or a part, fragment or functional form thereof and a reporter molecule. In each case, the expression of the reporter is operatively linked to the expression of the molecule to be reported.

Reference to “cellular phenotype” herein encompasses the molecular or functional characteristics of a cell. For example, ASC cells express Blimp-1 (a molecular marker) and are functionally distinguished from other B-cells by exhibiting, inter alia, a high rate of Ig secretion, the absence of MHC class II molecules and low levels of surface Ig. As used herein, the term is a reference to the full range of molecular or functional characteristics, or any particular molecules or functional characteristic in addition to the molecular characteristic of modulated levels of Blimp-1 expression.

The genetically modified cell or non-human organism comprising such cells may comprise cells or genetic material from any organism such as, but not limited to, humans, non- human primates, livestock, companion or laboratory test organism, reptilian or amphibian species. Preferably the genetically modified organism is a mouse or other laboratory test animal such as a rat, guinea pig, pig, rabbit or sheep.

As used herein the singular forms “a”, “an” and “the” include plural aspects unless the context clearly dictates otherwise. Thus, for example, reference to a “cell” includes a single cell, as well as two or more cells; reference to “a gene” includes a gene, as well as two or more gene; and so forth.

The modified gene of the present invention is a marker for terminal differentiation in cells of the haematopoietic system, such as B-cell lineage cells.

Reference to a “genetically modified cell” is a reference to any cell which has been engineered to comprise a sequence of nucleotides from a coding or non-coding region of the genome which is altered relative to its pre-modified form, and its progeny. In particular, the cell is genetically modified to co-express a genetic marker of terminal differentiation and a reporter molecule encoding sequence. Preferably, the cell is genetically modified to co-express Blimp or a part, fragment or functional part thereof and a reporter molecule. The reporter molecule may be any molecule capable of directly or indirectly providing an identifiable signal. A fluorescent or other light emitting reporter molecule is particularly preferred.

Conveniently, targeting constructs are initially used to generate the modified genetic sequences in the cell or organism. Targeting constructs generally but not exclusively modify a target sequence by homologous recombination. Alternatively, a modified genetic sequence may be introduced using artificial chromosomes. Targeting or other constructs are produced and introduced into target cells using methods well known in the art which are described in molecular biology laboratory manuals such as, for example, in Sambrook, Molecular Cloning: A Laboratory Manual, 3rd Edition, CSHLP, CSH, NY, 2001; Ausubel (Ed) Current Protocols in Molecular Biology, 5th Edition, John Wiley & Sons, Inc, NY, 2002. Targeting constructs may be introduced into cells by any method such as electroporation, viral mediated transfer or microinjection. Selection markers are generally employed to initially identify cells which have successfully incorporated the targeting construct. As the skilled artisan will appreciate, the subject modified organisms may be genetically modified to express the Blimp allele and reporter molecule in only certain cells.

In one particular embodiment the present invention provides a nucleic acid construct suitable for use as a targeting construct said construct comprising all or a portion of an allele of Blimp-1 and a reporter construct. The construct comprise genetic material which encodes a functionally active Blimp-1 polypeptide or a functionally inactive Blimp-1 polypeptide. In a particular embodiment, the construct encodes a partial Blimp-1 polypeptide which lacks a zinc finger domain comprising a DNA binding motif. In a particularly preferred embodiment, the construct is flanked by sites to facilitate recombinase mediated deletion and homologous recombination of the nucleic acid construct into a target genetic sequence. Alternatively, the construct may be introduced into a host cell where it replicates episomally.

Genetically modified organisms are generated using techniques well known in the art such as described in Hogan et al., Manipulating the Mouse Embryo: A Laboratory Manual, Cold Spring Harbour Laboratory Press, CSH NY, 1986; Mansour et al., Nature 336:348-352, 1988; Pickert, Transgenic Animal Technology: A Laboratory Handbook, Academic Press, San Diago, Calif., 1994. Stem cells including embryonic stem cells (ES cells) are introduced into the embryo of a recipient organism at the blastocyst stage of development. There they are capable of integration into the inner cell mass where they develop and contribute to the germ line of the recipient organism. ES cells are conveniently obtained from pre-implantation embryos maintained in vitro (Robertson et al., Nature 322:445-448, 1986). Once correct targeting has been verified, modified cells are injected into the blastocyst or morula or other suitable developmental stage, to generate a chimeric organism. Alternatively, modified cells are allowed to aggregate with dissociated embronic cells to form aggregation chimera. The chimeric organism is then implanted into a suitable female foster organism and the embryo allowed to develop to term. Chimeric progeny are bred to obtain offspring in which the genome of each cell contains the nucleotide sequences conferred by the targeting construct. Genetically modified organism may comprise a heterozygous modification or alternatively both alleles may be affected.

In accordance with the present invention it is surprisingly determined that Blimp-1 is essential for the production of antibody by ASC but not the commitment to differentiate down the ASC pathway. Accordingly, the identification of Blimp (eg via a reporter molecule co-expressed therewith) in B-cell lineage cells indicates that the cells are committed to differentiate or have differentiated into ASC.

Furthermore, as disclosed herein, Blimp is essential for lymphocyte homeostasis including T-cell homeostasis and the ability of T-cells to become terminally differentiated. The absence of Blimp in adult mammals leads to aggressive multi-organ lymphproliferative disease.

Accordingly, another aspect of the present invention provides a genetically modified cell or non-human organism comprising such cells comprising genetic material encoding a polypeptide which when expressed produces the polypeptide co-expressed with a reporter molecule wherein detection of said reporter molecule is indicative of a cellular phenotype and/or commitment of a cell to terminally differentiate.

In a further aspect, the present invention provides a genetically modified cell or non-human organism comprising such cells comprising a modified Blimp gene encoding a Blimp polypeptide which when expressed produces Blimp or a part, fragment or functional form thereof co-expressed with a reporter molecule and wherein detection of said reporter molecule is indicative of a cellular phenotype and/or commitment of a cell to terminally differentiate.

In a further preferred aspect, the present invention provides a genetically modified cell or non-human organism comprising such cells comprising a modified Blimp gene encoding a Blimp mRNA transcript comprising a Blimp coding sequence or a part, fragment or functional form thereof and a reporter molecule encoding sequence, wherein detection of said reporter molecule is indicative of a cellular phenotype and/or commitment of a cell to terminally differentiate

Preferably, the reporter molecule encoding sequence is inserted with an intron of the Blimp allele. In this way, the modified Blimp allele co-produces the reporter from a bicistronic RNA under the control of endogenous Blimp regulatory elements.

Accordingly, another aspect of the present invention provides a genetically modified cell or non-human organism comprising such cells comprising genetic material encoding a polypeptide which when expressed produces the polypeptide co-expressed with a reporter molecule and wherein detection of said reporter molecule in cells of the haematopoietic system is indicative of a cellular phenotype and/or commitment of a cell to terminally differentiate.

In a further aspect, the present invention provides a genetically modified cell or non-human organism comprising such cells comprising a modified Blimp gene encoding a Blimp polypeptide which when expressed produces Blimp or a part, fragment or functional form thereof co-expressed with a reporter molecule and wherein detection of said reporter molecule in B-cells is indicative that cells having reporter molecule activity are committed to differentiation into ASC.

In a further preferred aspect, the present invention provides a genetically modified cell or non-human organism comprising such cells comprising a modified Blimp gene encoding a Blimp mRNA transcript comprising a Blimp coding sequence or a part, fragment or functional form thereof and a reporter molecule encoding sequence, wherein detection of said reporter molecule in T-cells is indicative that cells having reporter molecule activity are activated T-cells.

Preferably, the reporter molecule encoding sequence is inserted with an intron of the Blimp allele. In this way, the modified Blimp allele co-produces the reporter from a bicistronic RNA under the control of endogenous Blimp regulatory elements.

Reference herein to a Blimp-1 gene or nucleic acid expression product thereof (RNA) includes homologs, parts, fragments, functional forms thereof including functional variants or derivatives which hybridize thereto under low stringency conditions or comprise significant sequence similarity to all or a functional part such as at least about 60% sequence similarity, after optimal alignment. Reference to a Blimp-1 polypeptide or protein is used in a broad sense to include all homologs, parts, fragments or functional forms thereof including functional variants or derivatives bearing at least about 60% amino acid sequence similarity after optimal alignment.

Functional parts of the instant molecules include portions of the full length molecule which are important for the particular functions thereof such as substrate binding, tertiary conformation or transcriptional activity. Transcription initiation sites are readily mapped and sites conferring promoter activity readily identified (see for example Tunyaplin et al., Nucleic Acid Research 28(24):4846-4855, 2000). Functional parts are important for regulating the expression and activity of the molecule. Functional variants or derivatives retain at least one of the functional activities important for regulating expression and activity of a reference molecule. With reference to Blimp-1, its expression is associated with terminal differentiation, induction of Ig secretion by ASC cells and activation of T-cells.

The modified Blimp gene may encode a functionally active Blimp polypeptide, a functionally inactive Blimp polypeptide and/or partial Blimp polypeptide such as a polypeptide or peptide, for example, lacking a zinc finger domain comprising a DNA binding motif. The terms “polypeptide” and “protein” are used interchangeably herein.

A “part” in peptide form may be as small as an epitope comprising less than 5 amino acids or as large as several hundred kilodaltons. The length of the polypeptide sequences compared for homology will generally be at least about 16 amino acids, usually at least about 20 residues, more usually at least about 24 residues, typically at least about 28 residues and preferably more than about 35 residues. A “part” of a nucleic acid molecule is defined a having a minimal size of at least about 10 nucleotides or preferably about 13 nucleotides or more preferably at least about 20 nucleotides and may have a minimal size of at least about 35 nucleotides. This definition includes all sizes in the range of 10-35 nucleotides as well as greater than 35 nucleotides including 50, 100, 300, 500, 600 nucleotides or nucleic acid molecules having any number of nucleotides within these values.

The present invention also contemplates modified Blimp alleles encoding variant Blimp polypeptides. “Variant” polypeptides include proteins derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is, they continue to possess the desired biological activity of the native protein (i.e, they are transcriptional repressors of for example c-myc and/or CIITA). Alternatively, the variant Blimp polypeptides are non-functional. Such variants may result from, for example, genetic polymorphism or from human manipulation. Biologically active variants of a native Blimp polypeptide will have at least 40%, 50%, 60%, 70%, generally at least 75%, 80%, 85%, preferably about 90% to 95% or more, and more preferably about 98% or more sequence similarity with the amino acid sequence for the native protein as determined by sequence alignment programs described elsewhere herein using default parameters. A biologically active variant of a Blimp polypeptide may differ from that polypeptide generally by as much 100, 50 or 20 amino acid residues or suitably by as few as 1-15 amino acid residues, as few as 1-10, such as 6-10, as few as 5, as few as 4, 3, 2, or even 1 amino acid residue.

A Blimp polypeptide may be altered in various ways including amino acid substitutions, deletions, truncations, and insertions. Methods for such manipulations are generally known in the art. For example, amino acid sequence variants of a Blimp polypeptide can be prepared by mutations in the encoding nucleic acid sequence. Methods for mutagenesis and nucleotide sequence alterations are well known in the art. See, for example, Kunkel (Proc. Natl. Acad. Sci. USA 82:488-492, 1985), Kunkel et al., (Methods in Enzymol. 154:367-382, 1987), U.S. Pat. No. 4,873,192, Watson et al. (“Molecular Biology of the Gene”, Fourth Edition, Benjamin/Cummings, Menlo Park, Calif., 1987) and the references cited therein. Guidance as to appropriate amino acid substitutions that do or do not affect biological activity of the protein of interest may be found in the model of Dayhoff et al., (Natl. Biomed. Res. Found 5:345-358, 1978). For example deletion of all or part of the zinc finger domains containing the DNA binding motif will produce a Blimp variant which is functionally inactive. In some embodiments, animal models are heterozygous in some or all tissues for a genetically modified non-functional Blimp allele, while the other allele comprises a functional Blimp allele capable of expressing a functional Blimp polypeptide. In other embodiments, homozygous animals are produced which do not express Blimp in particular cells or tissues. Alternatively functional Blimp may be produced by one or two modified Blimp alleles in a cell, tissue or non-human organism. Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of Blimp polypeptides. Recursive ensemble mutagenesis (REM), a technique which enhances the frequency of functional mutants in the libraries, can be used in combination with the screening assays to identify Blimp polypeptide variants (Arkin et al., Proc. Natl. Acad. Sci. USA 89:7811-7815, 1992; Delgrave et al. Protein Engineering 6:327-331, 1993). Conservative substitutions, such as exchanging one amino acid with another having similar properties, may be desirable as discussed in more detail below.

Variant Blimp polypeptides may contain conservative amino acid substitutions at various locations along their sequence, as compared to the parent Blimp amino acid sequence. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterises certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al., 1978 (supra); and by Gonnet et al., Science 256(5062):1443-1445, 1992), however, include proline in the same group as glycine, serine, alanine and threonine. Accordingly, for the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behaviour.

Amino acid residues can be further sub-classified as cyclic or noncyclic, and aromatic or nonaromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always nonaromatic. Dependent on their structural properties, amino acid residues may fall in two or more classes. For the naturally-occurring protein amino acids, sub-classification according to this scheme is presented in the Table A.

TABLE A
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence | |
| chain orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional Blimp polypeptide can readily be determined by assaying its activity. Conservative substitutions are shown in Table B below under the heading of exemplary substitutions. More preferred substitutions are shown under the heading of preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE B
Exemplary and Preferred Amino Acid Substitutions

| Orginal | | PREFERRED |
|---|---|---|
| Residue | EXEMPLARY SUBSTITUTIONS | SUBSTITUTIONS |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

Alternatively, similar amino acids for making conservative substitutions can be grouped into three categories based on the identity of the side chains. The first group includes glutamic acid., aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm. C. Brown Publishers (1993).

Thus, a predicted non-essential amino acid residue in a Blimp polypeptide is typically replaced with another amino acid residue from the same side chain family. Alternatively, mutations can be introduced randomly along all or part of a Blimp polynucleotide coding sequence, such as by saturation mutagenesis, and the resultant mutants can be screened for an activity of the parent polypeptide to identify mutants which retain that activity. Following mutagenesis of the coding sequences, the encoded peptide can be expressed recombinantly and the activity of the peptide can be determined.

Accordingly, the present invention also contemplates variants of the naturally-occurring Blimp polypeptide sequences or their biologically-active fragments, wherein the variants are distinguished from the naturally-occurring sequence by the addition, deletion, or substitution of one or more amino acid residues. In general, variants will display at least about 30, 40, 50, 55, 60, 65, 70, 75, 80, 85, 90, 91, 92, 93, 94, 95, 96, 97, 98, 99% similarity to a parent Blimp polypeptide sequence as, for example, set forth in any one of SEQ ID NO: 2 and 4. Desirably, variants will have at least 30, 40, 50, 55, 60, 65, 70, 75, 80, 85, 90, 91, 92, 93, 94, 95, 96, 97, 98, 99% sequence identity to a reference Blimp polypeptide sequence as, for example, set forth in any one of SEQ ID NO: 2 and 4. Moreover, sequences differing from the native or parent sequences by the addition, deletion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100 or more amino acids but which retain the properties of the parent Blimp polypeptide are contemplated. Blimp polypeptides also include polypeptides that are encoded by polynucleotides that hybridise under stringency conditions as defined herein, especially high stringency conditions, to Blimp polynucleotide sequences, or the non-coding strand thereof.

In some embodiments, variant polypeptides differ from an Blimp sequence by at least one but by less than 50, 40, 30, 20, 15, 10, 8, 6, 5, 4, 3 or 2 amino acid residue(s). In another, variant polypeptides differ from the corresponding sequence in any one of SEQ ID NO: 2 and 4 by at least 1% but less than 20%, 15%, 10% or 5% of the residues, (If this comparison requires alignment the sequences should be aligned for maximum similarity. “Looped” out sequences from deletions or insertions, or mismatches, are considered differences.) The differences are, suitably, differences or changes at a non-essential residue or a conservative substitution.

A “non-essential” amino acid residue is a residue that can be altered from the wild-type sequence of an embodiment polypeptide without abolishing or substantially altering one or more of its activities. Suitably, the alteration does not substantially alter one of these activities, for example, the activity is at least 20%, 40%, 60%, 70% or 80% of wild-type. An “essential” amino acid residue is a residue that, when altered from the wild-type sequence of an Blimp polypeptide of the invention, results in abolition of an activity of the parent molecule such that less than 20% of the wild-type activity is present.

In other embodiments, a variant polypeptide includes an amino acid sequence having at least about 50%, 55%, 60%, 65%, 70%, 75%, 80%, 85%, 90%, 91%, 92%, 93%, 94% 95%, 96%, 97%, 98% or more similarity to a corresponding sequence of a Blimp polypeptide as, for example, set forth in any one of SEQ ID NO: 2 and 4.

The present invention encompasses Blimp from any mammal or animal (including avian species) subject such as from humans, non-human primates, livestock, laboratory, companion or wild animals. Reference to “Blimp” includes Blimp or Blimp from any of the above species as well as structural or evolutionary equivalents or homologs thereof. for example, the present invention encompasses Blimp or a Blimp having an amino acid sequence which has substantially at least about 60% similarity to SEQ ID NO: 2 or 4 or at least about 60% identity to SEQ ID NO: 1, 3, 5 or 6. Reference to at least about 60% includes 60, 61, 62, 63, 64% and all following consecutive numbers in the series to 100%.

Function derivatives of molecules in nucleic acid form include nucleic acid molecules comprising a nucleotide sequence capable of hybridising to the molecule or its complementary form under low stringency conditions.

The terms “similarity” or identity as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and amino acid sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al., Nucl. Acids Res. 25: 3389, 1997. A detailed discussion of sequence analysis can be found in Unit 19.3 of Ausubel et al., Current Protocols in Molecular Biology John Wiley & Sons Inc, 1994-1998, Chapter 15).

The terms “sequence similarity” and “sequence identity” as used herein refer to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Furthermore, a Blimp homolog or derivative may be defined as being capable of hybridising to SEQ ID NO: 1, 3, 5 or 6 or to a complementary form thereof under low stringency conditions.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C)% (Marmur et al., J. Mol. Biol. 5: 109, 1962). However, the Tₘ of a duplex DNA decreases by 1° C. with every increase of 1% in the number of mismatch base pairs (Bonner et al., Eur. J. Biochem. 46: 83, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

Preferably the modified Blimp gene is modified using a nucleic acid construct comprising all or a portion of an allele of Blimp into which a nucleotide sequence encoding a reporter molecule is inserted.

The reporter molecule is conveniently encoded by a reporter expression cassette or reporter construct. The reporter construct can be brought under the control of the Blimp-1 regulatory elements and faithfully report the Blimp-1 expression pattern in cells, tissues or organisms.

By “reporter” is meant any molecule, protein or polypeptide which is typically encoded by a reporter gene and measured in a reporter assay. Reporters provide a detectable signal which permit an understanding of the activity of genetic sequences. They may report an activity directly or may indirectly monitor activity by monitoring the activity of down stream targets. A reporter protein should be distinguishable from other proteins and ideally, readily quantified. The reactivity between an epitope and an antibody determined thereby may readily be employed optionally together with second or further antibodies. Common reporter proteins include luciferase, chloramphenicol transferase (CAT), Beta-galactosidase (B-gal), or fluorescent proteins such as green fluorescent proteins (GFP). Reference herein to GFP is meant to encompass any fluorescent or light-emitting protein including those derived from jellyfish or other organisms and all homologues, derivatives, analogues including colour variants such as DSRed, HcRed, Clontech; or hrGFP, Stratagene). Preferably said reporter expression cassette encodes a fluorescent or other light emitting GFP. GFP reporters are readily detectable in live cells and are particularly useful and preferred in cell sorting applications.

Examples of fluorescent or light emitting markers may be selected from among those included, but are not limited to those, in the following Table 2.

TABLE 2

| | | |
|---|---|---|
| Probe | Ex¹ (nm) | Em² (nm) |

Reactive and conjugated probes

| | | |
|---|---|---|
| Hydroxycoumarin | 325 | 386 |
| Aminocoumarin | 350 | 455 |
| Methoxycoumarin | 360 | 410 |
| Cascade Blue | 375; 400 | 423 |
| Lucifer Yellow | 425 | 528 |
| NBD | 466 | 539 |
| R-Phycoerythrin (PE) | 480; 565 | 578 |
| PE-Cy5 conjugates | 480; 565; 650 | 670 |
| PE-Cy7 conjugates | 480; 565; 743 | 767 |
| APC-Cy7 conjugates | 650; 755 | 767 |
| Red 613 | 480; 565 | 613 |
| Fluorescein | 495 | 519 |
| FluorX | 494 | 520 |
| BODIPY-FL | 503 | 512 |
| TRITC | 547 | 574 |
| X-Rhodamine | 570 | 576 |
| Lissamine Rhodamine B | 570 | 590 |
| PerCP | 490 | 675 |
| Texas Red | 589 | 615 |
| Allophycocyanin (APC) | 650 | 660 |
| TruRed | 490, 675 | 695 |
| Alexa Fluor 350 | 346 | 445 |
| Alexa Fluor 430 | 430 | 545 |
| Alexa Fluor 488 | 494 | 517 |
| Alexa Fluor 532 | 530 | 555 |
| Alexa Fluor 546 | 556 | 573 |
| Alexa Fluor 555 | 556 | 573 |
| Alexa Fluor 568 | 578 | 603 |
| Alexa Fluor 594 | 590 | 617 |
| Alexa Fluor 633 | 621 | 639 |
| Alexa Fluor 647 | 650 | 688 |
| Alexa Fluor 660 | 663 | 690 |
| Alexa Fluor 680 | 679 | 702 |
| Alexa Fluor 700 | 696 | 719 |
| Alexa Fluor 750 | 752 | 779 |
| Cy2 | 489 | 506 |
| Cy3 | (512); 550 | 570; (615) |
| Cy3,5 | 581 | 596; (640) |
| Cy5 | (625); 650 | 670 |
| Cy5,5 | 675 | 694 |
| Cy 7 | 743 | 767 |

Nucleic acid probes

| | | |
|---|---|---|
| Hoeschst 33342 | 343 | 483 |
| DAPI | 345 | 455 |
| Hoechst 33258 | 345 | 478 |
| SYTOX Blue | 431 | 480 |
| Chromomycin A3 | 445 | 575 |
| Mithramycin | 445 | 575 |
| YOYO-1 | 491 | 509 |
| SYTOX Green | 504 | 523 |
| SYTOX Orange | 547 | 570 |
| Ethidium Bormide | 493 | 620 |
| 7-AAD | 546 | 647 |
| Acridine Orange | 503 | 530/640 |
| TOTO-1, TO-PRO-1 | 509 | 533 |
| Thiazole Orange | 510 | 530 |
| Propidium Iodide (PI) | 536 | 617 |
| TOTO-3, TO-PRO-3 | 642 | 661 |
| LDS 751 | 543; 590 | 712; 607 |

Cell function probes

| | | |
|---|---|---|
| Indo-1 | 361/330 | 490/405 |
| Fluo-3 | 506 | 526 |
| DCFH | 505 | 535 |
| DHR | 505 | 534 |
| SNARF | 548/579 | 587/635 |

Fluorescent Proteins

| | | |
|---|---|---|
| Y66F | 360 | 508 |
| Y66H | 360 | 442 |
| EBFP | 380 | 440 |
| Wild-type | 396, 475 | 50, 503 |
| GFPuv | 385 | 508 |
| ECFP | 434 | 477 |
| Y66W | 436 | 485 |
| S65A | 471 | 504 |
| S65C | 479 | 507 |
| S65L | 484 | 510 |
| S65T | 488 | 511 |
| EGFP | 489 | 508 |
| EYFP | 514 | 527 |
| DsRed | 558 | 583 |

Other probes

| | | |
|---|---|---|
| Monochlorobimane | 380 | 461 |
| Calcein | 496 | 517 |

¹Ex: Peak excitation wavelength (nm)
²2Em: Peak emission wavelength (nm)

Any suitable method of analyzing fluorescence emission is encompassed by the present invention. In this regard, the invention contemplates techniques including but not restricted to 2-photon and 3-photon time resolved fluorescence spectroscopy as, for example, disclosed by Lakowicz et al., Biophys. J. 72: 567, 1997, fluorescence lifetime imaging as, for example, disclosed by Eriksson et al., Biophys. J. 2: 64, 1993, incorporated herein by reference) and fluorescence resonance energy transfer as, for example, disclosed by Youvan et al., Biotechnology et Elia 3: 1-18, 1997).

Exemplary fluorophores which may be used in accordance with the present invention include those discussed by Dower et al. (International Patent Publication No. WO 93/06121). Preferably, fluorescent dyes are employed. Any suitable fluorescent dye may be used for incorporation into the instant reporter molecule. For example, reference may be made to U.S. Pat. No. 5,573,909 (Singer et al.) and U.S. Pat. No. 5,326,692 (Brinkley et al.) which describe a plethora of fluorescent dyes. Reference may also be made to fluorescent dyes described in U.S. Pat. Nos. 5,227,487, 5,274,113, 5,405,975, 5,433,986, 5,442,045, 5,451,663, 5,453,517, 5,459,276, 5,515,864, 5,648,270 and 5,723,218.

A modern flow cytometer is able to perform these tasks up to 100,000 cells/particles s⁻¹. Through the use of an optical array of filters and dichroic mirrors, different wavelengths of fluorescent light can be separated and detected simultaneously. In addition, a number of lasers with different excitation wavelengths may be used. Hence, a variety of fluorophores can be used to target and examine, for example, intra- and extra-cellular properties of individual cells. The scattered light measurements can also classify an individual cells's size, shape, granularity and/or complexity and, hence, belonging to a particular population of interest (Shapiro, Practical flow cytometry, 3^{rd} Ed., Brisbane, Wiley-Liss, 1995).

Suitable flow cytometers which may be used in the methods of the present invention include those which measure five to nine optical parameters (see Table 3) using a single excitation laser, commonly an argon ion air-cooled laser operating at 15 mW on its 488 nm spectral line. More advanced flow cytometers are capable of using multiple excitation lasers such as a HeNe laser (633 nm) or a HeCd laser (325 nm) in addition to the argon ion laser (488 or 514 nm). Optical parameters, corresponding to different optically detectable/quantifiable attributes, for a carrier, may be measured by a flow cytometer to provide a matrix of qualitative and/or quantitative information, providing a code (or addressability in a multi-dimensional space) for the carrier.

For example, Biggs et al. (Cytometry 36: 36-45, 1999) have constructed an 11-parameter flow cytometer using three excitation lasers and have demonstrated the use of nine distinguishable fluorophores in addition to forward and side scatter measurements for purposes of immunophenotyping (i.e. classifying) cells. The maximum number of parameters commercially available currently is 17: forward scatter, side scatter and three excitation lasers each with five fluorescence detectors. Whether all of the parameters can be adequately used depends heavily on the extinction coefficients, quantum yields and amount of spectral overlap between all fluorophores (Malemed et al., “Flow cytometry and sorting”, 2^{nd} Ed., New York, Wiley-Liss, 1990). However, it will be understood that the present invention is not restricted to any particular flow cytometer or any particular set of parameters. In this regard, the invention also contemplates use in place of a conventional flow cytometer, a microfabricated flow cytometer as, for example., disclosed by Fu et al., Nature Biotechnology 17: 1109-1111, 1999.

TABLE 3
Exemplary optical parameters which may be measured
by a flow cytometer.

| | | Detection angle form | Wavelength |
|---|---|---|---|
| Parameter | Acronym | incident laser beam | (nm) |
| Forward scattered light | FS | 2-5° | 488* |
| Side scattered light | SS | 90° | 488* |
| “Green” fluorescence | FL1 | 90° | 510-540^{†} |
| “Yellow” fluorescence | FL2 | 90° | 560-580^{†} |
| “Red” fluorescence | FL3 | 90° | >650^{#} |

*using a 488 nm excitation laser
^{†}width of bandpass filter
^{#}longpass filter

A flow cytometer with this capacity to sort is known as a “fluorescence-activated cell sorter” (FACS). Accordingly, the step of sorting in the present method of obtaining a population of detectably unique carriers may be effected by flow cytometric techniques such as by fluorescence activated cell sorting (FACS) although with respect to the present invention, FACS is more accurately “fluorescence activated carrier or solid support sorting” (see, for example, “Methods in Cell Biology” Vol. 33, Darzynkiewica, Z. and Crissman, H. A., eds., Academic Press).

In a further embodiment the present invention provides a method for phenotyping and/or monitoring a cell of the haematopoietic system comprising screening a genetically modified cell or non-human organism comprising such cells comprising a modified Blimp gene wherein expression or activity of said gene is indicative of a cellular phenotype and/or a commitment of said cell to terminally differentiate. Haematopoietic cells include but are not limited to B-cells, T-cells, dendritic cells, macrophages and natural killer cells, granulocytes, eosinophils, erythrocytes, megakaryocytes, bone marrow, stromal, splenic precursor cells and their derivatives.

Preferably the modified Blimp gene encodes a Blimp mRNA transcript comprising a Blimp coding sequence or a part, fragment or functional form thereof and a reporter molecule encoding sequence which when expressed produces Blimp or a part, fragment or functional form thereof co-expressed with a reporter molecule and wherein detection of the reporter molecule is indicative of cellular phenotype and/or commitment of a cell to terminally differentiate.

In a further embodiment, cells which exhibit reporter activity or changes in reporter activity are isolated or selected from among cells which do not exhibit reporter activity. Isolation of reporter-active cells may be by flow cytometry, laser scanning cytometry, chromatography and/or other equivalent procedures. Additionally, further selection markers may be used to isolate or select the modified cells of the present invention. Flow cytometric isolation is particularly preferred.

Preferably the cells are ASC identified or isolated in a population of cells of a B-cell lineage.

Accordingly, the present invention provides a method for isolating a substantially purified population of ASC from a population of substantially B-cells said method comprising contacting a genetically modified cell or non-human organism comprising such cells comprising a modified Blimp gene with an agent or composition capable of inducing differentiation to ASC wherein expression or activity of said gene is reported by a reporter construct and wherein detection of said reporter activity is indicative that cells with reporter molecule activity are ASC, where necessary isolating B-cells from said organism and isolating ASC based on the activity of the reporter molecule.

Preferably the modified cell comprises a modified Blimp gene encoding a Blimp mRNA transcript comprising a Blimp coding sequence or a part, fragment or functional form thereof and a reporter molecule encoding sequence which when expressed produces Blimp or a part, fragment or functional form thereof co-expressed with a reporter molecule and wherein reporter activity is indicative that cells with reporter molecule activity are ASC.

Preferably, screening of cells is achieved by flow cytometric analysis of a fluroescent reporter molecule.

B-cells are conveniently isolated from an organism or sample for example by density gradient centrifugation, flow cytometry or using magnetic beads. Any agent or composition which selectively, clonally or polyclonally of otherwise effectively activates B-cells and induces their differentiation to ASC is encompassed. An example of a polyclonal activator is LPS.

In one embodiment the reporter is a GFP and said ASC are isolated by flow cytometry.

Substantially purified means that the ASC comprise at least about 60 to 95%, preferably at least about 97%, more preferably at least about 99% of the cells, such as at least about 60, 61, 62, 63, 64 and following subsequent numbers in the series to 100%. Alternatively, enrichment of approximately 100,000 fold over unsorted cells is contemplated.

The present invention also provides a method for testing the antigenicity of a vaccine or the ability of agents to enhance or suppress antibody production by ASC wherein reduced reporter activity is indicative of an agent which down regulates or inhibits an antibody response and reporter activity or enhanced reporter activity relative to controls is indicative of agents which are positive regulators of the antibody response. In accordance with this aspect, the method comprises:
(i) administering an agent or vaccine to a genetically modified cell or non-human animal comprising such cells wherein the cell or organism comprises a modified Blimp-1 gene which encodes a Blimp polypeptide which when expressed produces Blimp or a part or fragment or functional form thereof co-expressed with a reporter molecule;
(ii) testing the cell or organism for the reporter molecule the presence of which is indicative of cellular phenotype and the ability of said agent or vaccine to regulate antibody production by ASC.

In another embodiment, the present invention provides a method for testing the antigenicity or immunogenicity of a vaccine comprising a genetic or proteinaceous composition, the method comprising;
(i) administering the vaccine to a genetically modified cell or non-human animal comprising such cells wherein the cell or organism comprises a modified Blimp-1 gene which encodes a Blimp polypeptide which when expressed produces Blimp or a part or fragment or functional form thereof co-expressed with a reporter molecule; and
(ii) testing the cell or organism for the reporter molecule the presence of which is indicative of the ability of the vaccine to regulate the activation of T-cells and/or B-cells.

In some embodiments, the Blimp gene encodes a Blimp mRNA transcript comprising a Blimp coding sequence or a part, fragment or functional form thereof and a reporter molecule coding sequence. In other embodiments, the reporter molecule coding sequence is inserted within an intron of a Blimp allele. In further embodiments, the modified Blimp allele is present in homozygous or heterozygous form. Depending upon the purpose of the assay in some embodiments, the modified Blimp allele encodes a functional Blimp transcription factor or a functional part, form, homolog or variant thereof. In other embodiments, the modified Blimp allele encodes a non-functional Blimp transcription factor or a non-functional part, form, homolog or variant thereof. In an illustrative embodiment, the cells or genetic material are derived from man, a non-human primate, a livestock, companion or laboratory test organisms, reptilian or amphibian species. Examples of laboratory test animal include a rodent (including mice), guinea pig, pig, duck, rabbit or sheep.

In some illustrative embodiments of the methods disclosed herein the cell is a haematopoietic or embryonic cell. As disclosed herein Blimp is essential for both B-cell and T-cell terminal differentiation and accordingly a preferred cell lineage is is a lymphocytic cell. In particular embodiments the lymphocytic cell types is selected from a B-cell and a T-cell. Where a B-cell, the terminally differentiated form is an ASC and these cell can furthermore be substantially purified using the methods disclosed herein. When the cell is a B-cell, the terminally differentiated T-cells include without limitation CD40⁺ T-cells and CD8⁺ T-cells. Conveniently, the detection of the reporter molecule is indicative or predictive of a cellular phenotype and/or commitment of a cell to terminally differentiate under particular conditions or in the presence of test agents. Still more conveniently, the reporter molecule is a fluorescent or light emitting reporter molecule.

The present invention also directed to antagonists and agonists of terminal differentiation of cells such as, but not limited to ASC including antagonists and agonists of Blimp-1 expression or Blimp-1 activity, identified by the herein described method, for use in modulating cellular differentiation, The molecules to which the instant modulators, agonists or antagonists are directed are collectively referred to herein as “targets” or “target molecules”.

In another aspect therefore, the present invention provides methods for in vitro or in vivo screening for agonists or antagonists of terminal differentiation in haematopoietic cells comprising exposing one or more agent/s to a genetically modified cell or non-human animal comprising such cells wherein the cell or organism comprises a modified Blimp-1 gene which encodes a Blimp polypeptide which when expressed produces Blimp or a part or fragment or functional form thereof co-expressed with a reporter molecule; and testing the cell or organism for the presence or a change in the level of the reporter molecule the presence of which is indicative of the ability of the one or more agent/s to agonise or antagonise terminal differentiation. Agonists of Blimp directly or indirectly induce terminal differentiation of haematopoietic cells and are useful, for example, in the treatment or prevention of cancer and/or autoimmune disease and in promoting appropriate immune responses to pathological infections. Molecules which inhibit generation of terminally differentiated cells are useful in autoimmune patents such as lupus patients or in treating immune dysfunction such as cases of allegy.

Preferably the modified cell is a haematopoietic cell which comprises a modified Blimp gene encoding a Blimp mRNA transcript comprising a Blimp coding sequence or a part, fragment or functional form thereof and a reporter molecule encoding sequence which when expressed produces Blimp or a part, fragment, homolog, variant, derivative or functional or non-functional form thereof co-expressed with a reporter molecule and wherein reporter activity is indicative that cells with reporter molecule activity are terminally differentiated or committed to terminal differentiation. More preferably, the haematopoietic cell is a lymphocyte lineage cell. In some embodiments the terminally differentiated cells are ASC: in other embodiments, the terminally differentiated cells are CD4 T-cell and/or CD8 T-cells. The modified Blimp allele is present in the cell, tissue or non-human organism in homozygous or heterozygous form. Furthermore, depending upon the particular application, the Blimp allele expresses a transcriptionally (functionally) active Blimp polypeptide. Thus in some assays it will be useful to have a functional Blimp polypeptide to modulate or induce terminal differentiation in a cell. In other embodiments, the modified Blimp allele does not express a functional Blimp and it will be sufficient to determine, via detection of the reporter activity whether a Blimp allele would have been expressed or whether the level of Blimp expression would have been modulated in a cell capable of producing a function Blimp polypeptide.

Cellular (in vitro) assays are particularly convenient and, when coupled with a reporter molecule whose activity can readily be detected in cells, the assays are ideally suited to high throughput screening. A large number of different formats are available as known to the skilled artisan. One useful example is described in Ulleras et al., Toxicology 206(2):245-256, 2005.

“Modulation” of a molecule or differentiation status includes completely or partially inhibiting or reducing or down regulating all or part of its functional activity or differentiation and enhancing or up regulating all or part its functional activity or differentiation. Where the molecule is a genetic sequence its functional activity may be modulated by, for example, modulating its binding capabilities or transcriptional or translational activity, or its half-life. Where the molecule is an encoded polypeptide, its functional activity may be modulated by, for example, modulating its binding capabilities, its half-life, location in a cell or membrane or its enzymatic capability. Modulators are agonists or antagonists which achieve modulation. Enhanced differentiation can also be indicative of reduced cell division.

An example of an antagonist or agonist is a protein, polypeptide or peptide. These terms may be used interchangeably. These terms refer to a polymer of amino acids and its equivalent and does not refer to a specific length of the product, thus, polypeptides, peptides, oligopeptides and proteins are included within the one definition of a polypeptide. These terms also do not exclude modifications of the polypeptide, for example, glycosylations, aceylations, phosphorylations and the like. Included within the definition are, for example, polypeptides containing one or more analogs of an amino acid including, for example, unnatural amino acids such as those given in Table 4 or polypeptides with substituted linkages. Such polypeptides may need to be able to enter the cell. Polypeptides carrying chemical analogs of the amino acids may be more resistant to protease mediated digestion. One example of an antagonist or agonist is a chemical analog of Blimp. Antagonists and agonists may affect the molecules with which Blimp interacts, such as, for example c-myc expression is repressed by Blimp-1.

Genetic molecules are also developed into agonist and antagonist modulators. The terms “genetic molecule” “nucleic acids”, “nucleotide” and “polynucleotide” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog (such as the morpholine ring), internucleotide modifications such as uncharged linkages (e.g. methyl phosphonates, phosphotriesters, phosphoamidates, carbamates, etc.), charged linkages (e.g. phosphorothioates, phosphorodithioates, etc.), pendent moieties (e.g. polypeptides), intercalators (e.g. acridine, psoralen, etc.), chelators, alkylators and modified linkages (e.g. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule. Modifications of antisense molecules are well known and are summarised in Kurrek, Eur. J. Biochem. 270:1628-1644, 2003.

Antisense polynucleotide sequences, for example, are useful in silencing transcripts. Furthermore, polynucleotide vectors containing all or a part of a Blimp gene locus may be placed under the control of a promoter in an antisense orientation and introduced into a cell. Expression of such an antisense construct within a cell will interfere with target transcription and/or translation. Such molecules may be particularly useful in dampening the immune response in autoimmune conditions. Furthermore, co-suppression and mechanisms to induce RNAi or siRNA may also be employed. Alternatively, antisense or sense molecules may be directly administered. In this latter embodiment, the antisense or sense molecules may be formulated in a composition and then administered by any number of means to target cells.

A variation on antisense and sense molecules involves the use of morpholinos, which are oligonucleotides composed of morpholine nucleotide derivatives and phosphorodiamidate linkages (for example, Summerton and Weller, Antisense and Nucleic Acid Drug Development 7: 187-195, 1997). Such compounds are injected into embryos and the effect of interference with mRNA is observed.

In one embodiment, the present invention employs compounds such as oligonucleotides and similar species for use in modulating the function or effect of nucleic acid molecules encoding Blimp i.e. the oligonucleotides induce transcriptional or post-transcriptional gene silencing. This is accomplished by providing oligonucleotides which specifically hybridize with one or more nucleic acid molecules encoding the endogenous ligands. The oligonucleotides may be provided directly to a cell or generated within the cell. As used herein, the terms “target nucleic acid” and “nucleic acid molecule encoding an inhibitor” have been used for convenience to encompass DNA encoding the inhibitor, RNA (including pre-mRNA and mRNA or portions thereof) transcribed from such DNA, and also cDNA derived from such RNA. The hybridization of a compound of the subject invention with its target nucleic acid is generally referred to as “antisense”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. In one example, the result of such interference with target nucleic acid function is reduced levels of Blimp. In the context of the present invention, “modulation” and “modulation of expression” mean either an increase (stimulation) or a decrease (inhibition) in the amount or levels of a nucleic acid molecule encoding the gene, e.g., DNA or RNA. Inhibition is often the preferred form of modulation of expression and mRNA is often a preferred target nucleic acid.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e. under physiological conditions in the case of in vivo assays or therapeutic treatment, and under conditions in which assays are performed in the case of in vitro assays.

“Complementary” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

According to the present invention, compounds include antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these compounds may be introduced in the form of single-stranded, double-stranded, circular or hairpin oligomeric compounds and may contain structural elements such as internal or terminal bulges or loops. Once introduced to a system, the compounds of the invention may elicit the action of one or more enzymes or structural proteins to effect modification of the target nucleic acid. One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While the preferred form of antisense compound is a single-stranded antisense oligonucleotide, in many species the introduction of double-stranded structures, such as double-stranded RNA (dsRNA) molecules, has been shovel to induce potent and specific antisense-mediated reduction of the function of a gene or its associated gene products. This phenomenon occurs in both plants and animals.

In the context of the subject invention, the term “oligomeric compound” refers to a polymer or oligomer comprising a plurality of monomeric units. In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

While oligonucleotides are a preferred form of the compounds of this invention, the present invention contemplates other families of compounds as well, including but not limited to oligonucleotides, analogs and mimetics such as those herein described.

The open reading frame (ORF) or “coding region” which is known in the art to refer to the region between the translation initiation codon and the translation termination codon, is a region which may be effectively targeted. Within the context of the present invention, one region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may, therefore, fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

The isolated or recombinant agonists and antagonists of the instant invention are used directly or they may be further modified by methods well known in the art in order to improve their effectiveness as pharmaceutical or other reagents. Important considerations for an active compound include formulations and methods of delivery.

An agonist or antagonist includes molecules determined by all or part of the target in genetic or proteinaceous form, such as antibodies, mimetics or antisense molecules.

Antibodies including anti-idiotypic antibodies, chaemeric antibodies and humanised antibodies are useful in this regard and their generation is now routine to those of skill in the art. Peptide or non-peptide mimetics can be developed as agonists of the targets by identifying those residues of the target molecule which are important for function. Modelling can be used to design molecules which interact with the target molecule and which have improved pharmacological properties. All such molecules will need to be modified to permit entry into a cell.

Rational drug design permits the production of structural analogs of biologically active polypeptides of interest or of small molecules with which they interact (e.g. agonists, antagonists, inhibitors or enhancers) in order to fashion drugs which are, for example, more active or stable forms of the polypeptide, or which, e.g. enhance or interfere with the function of a polypeptide in vivo. See, e.g. Hodgson (Bio/Technology 9: 19-21, 1991). In one approach, one first determines the three-dimensional structure of a protein of interest by x-ray crystallography, by computer modeling or most typically, by a combination of approaches. Useful information regarding the structure of a polypeptide may also be gained by modeling based on the structure of homologous proteins. An example of rational drug design is the development of HIV protease inhibitors (Erickson et al., Science 249: 527-533, 1990). In addition, target molecules may be analyzed by an alanine scan (Wells, Methods Enzymol. 202: 2699-2705, 1991). In this technique, an amino acid residue is replaced by Ala and its effect on the peptide's activity is determined. Each of the amino acid residues of the peptide is analyzed in this manner to determine the important regions of the peptide.

It is also possible to isolate a target-specific antibody, selected by a functional assay and then to solve its crystal structure. In principle, this approach yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacore.

Analogs contemplated herein include but are not limited to modification to side chains, incorporating of unnatural amino acids and/or their derivatives during peptide, polypeptide or protein synthesis and the use of crosslinkers and other methods which impose conformational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriplhenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 4.

TABLE 4
Codes for non-conventional amino acids

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylomithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutarnate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | MnLe |
| L-α-methylnorvaline | Mnva | L-α-methyloorithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyL) | Nnbhe |
| carbarnylmethyl)glycine | carbamylmethyl)glycine | | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids and the introduction of double bonds between C_{α} and C_{β} atoms of amino acids.

Natural product, combinatorial or phage display technologies are all available for screening for modulators. A huge choice of high through put screening methods are available which may be adapted to employ the cells of the present invention.

Two-hybrid screening is also useful in identifying other members of the genetic network acting with of Blimp-1. Target interactions and screens for modulators can be carried out using the yeast two-hybrid system, which takes advantage of transcriptional factors that are composed of two physically separable, functional domains. The most commonly used is the yeast GAL4 transcriptional activator consisting of a DNA binding domain and a transcriptional activation domain. Two different cloning vectors are used to generate separate fusions of the GAL4 domains to genes encoding potential binding proteins. The fusion proteins are co-expressed, targeted to the nucleus and if interactions occur, activation of a reporter gene (e.g. lacZ) produces a detectable phenotype. In the present case, for example, S. cerevisiae is co-transformed with a library or vector expressing a cDNA GAL4 activation domain fusion and a vector expressing a Myb pathway component fused to GAL4. If lacZ is used as the reporter gene, co-expression of the fusion proteins will produce a blue colour. Small molecules or other candidate compounds which interact with a target will result in loss of colour of the cells. Reference may be made to the yeast two-hybrid systems as disclosed by Munder et al., (Appl. Microbiol. Biotechnol. 52(3): 311-320, 1999) and Young et al., Nat. Biotechnol. 16(10): 946-950, 1998). Molecules thus identified by this system are then re-tested in the genetically modified organisms or genetically modified cells of the present invention,

Pharmaceutical compositions for therapy are further contemplated comprising recombinant, synthetic or isolated forms of the present agonists and antagonists and one or more pharmaceutically acceptable carriers, diluents or excipients. The treatment of cancer or the modulation of an immune response are particularly contemplated.

The term therapy should be taken as a reference to treatment or prophylaxis of a condition or disease. The term “treating” and “ameliorating” are used interchangeably.

The terms “composition” or “agent” or “medicament” refer to a chemical compound that induces a desired pharmacological and/or physiological effect. The term also encompass pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “compound” is not to be construed narrowly but extends to peptides, polypeptides and proteins as well as genetic molecules such as RNA, DNA and mimetics and chemical analogs thereof.

The phrases “ameliorating a disease or condition” or “treatment” or “therapeutic” are used in the broadest context and include any measurable or statistically significant improvement in a disease or condition or one or more symptoms or frequency of symptoms of a disease or condition as well as complete recovery from the disease or elimination of a condition, its symptoms or its underlying cause. The present invention is applicable to a large range of diseases or conditions and the skilled addressee must determine the precise parameters of the assessment of phenotypes on a case by case basis. Conditions may be associated with one or more diseased or they may not be so linked. The amelioration of a condition encompasses any desired physiological or psychological change.

An effective amount of the instant compositions is established best by those skilled in the art. The term “effective amount” of a compound as used herein mean a sufficient amount of the agent to provide the desired therapeutic or physiological effect. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

Pharmaceutical compositions for therapy are further contemplated comprising recombinant, synthetic or isolated forms of the present agonists and antagonists and one or more pharmaceutically acceptable carriers, diluents or excipients. The treatment of cancer or the modulation of an immune response are particularly contemplated.

The term therapy should be taken as a reference to treatment or prophylaxis of a condition or disease. The term “treating” and “ameliorating” are used interchangeably.

The terms “composition” or “agent” or “medicament” refer to a chemical compound that induces a desired pharmacological and/or physiological effect. The term also encompass pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “compound” is not to be construed narrowly but extends to peptides, polypeptides and proteins as well as genetic molecules such as RNA, DNA and mimetics and chemical analogs thereof.

The phrases “ameliorating a disease or condition” or “treatment” or “therapeutic” are used in the broadest context and include any measurable or statistically significant improvement in a disease or condition or one or more symptoms or frequency of symptoms of a disease or condition as well as complete recovery from the disease or elimination of a condition, its symptoms or its underlying cause. The present invention is applicable to a large range of diseases or conditions and the skilled addressee must determine the precise parameters of the assessment of phenotypes on a case by case basis. Conditions may be associated with one or more diseased or they may not be so linked. The amelioration of a condition encompasses any desired physiological or psychological change.

An effective amount of the instant compositions is established best by those skilled in the art. The term “effective amount” of a compound as used herein mean a sufficient amount of the agent to provide the desired therapeutic or physiological effect. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

The polypeptides, nucleic acids, antibodies, peptides, chemical analogs, agonists, antagonists or mimetics of the present invention can be formulated in pharmaceutic compositions which are prepared according to conventional pharmaceutical compounding techniques. See, for example, Remington's Pharmaceutical Sciences, 18^{th} Ed. (1990, Mack Publishing, Company, Easton, Pa., U.S.A.). The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. intravenous, oral, intrathecal, epineural or parenteral.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, International Patent Publication No. WO 96/11698.

For parenteral administration, the compound may dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered and the rate and time-course of administration will depend on the nature and severity of the condition being treated. Prescription of treatment, e.g. decisions on dosage, timing, etc. is within the responsibility of general practitioners or specialists and typically takes account of the disorder to be treated, the condition of the individual patient, the site of delivery, the method of administration and other factors known to practitioners. Examples of techniques and protocols can be found in Remington's Pharmaceutical Sciences, (supra).

Alternatively, targeting therapies may be used to deliver the active agent more specifically to certain types of cell, by the use of targeting systems such as antibodies or cell specific ligands. Targeting may be desirable for a variety of reasons, e.g. if the agent is unacceptably toxic or if it would otherwise require too high a dosage or if it would not otherwise be able to enter the target cells.

Instead of administering these agents directly, they could be produced in the target cell, e.g. in a viral vector such as described above or in a cell based delivery system such as described in U.S. Pat. No. 5,550,050 and International Patent Publication Nos, WO 92/19195, WO 94/25503, WO 95/01203, WO 95/05452, WO 96/02286, WO 96/02646, WO 96/40871, WO 96/40959 and WO 97/12635. The vector could be targeted to the target cells or expression of expression products could be limited to specific cells, stages of decelopment or cell cycle stages. The cell based delivery system is designed to be implanted in a patient's body at the desired target site and contains a coding sequence for the target agent. Alternatively, the agent could be administered in a precursor form for conversion to the active form by an activating agent produced in, or targeted to, the cells to be treated. See, for example, European Patent Application No. 0 425 731A and International Patent Publication No. WO 90/07936.

The present invention is further described by the following non-limiting Examples.

### EXAMPLE 1

### Generation of a Blimp-1 Mutant Allele (Blimp^{gfp})

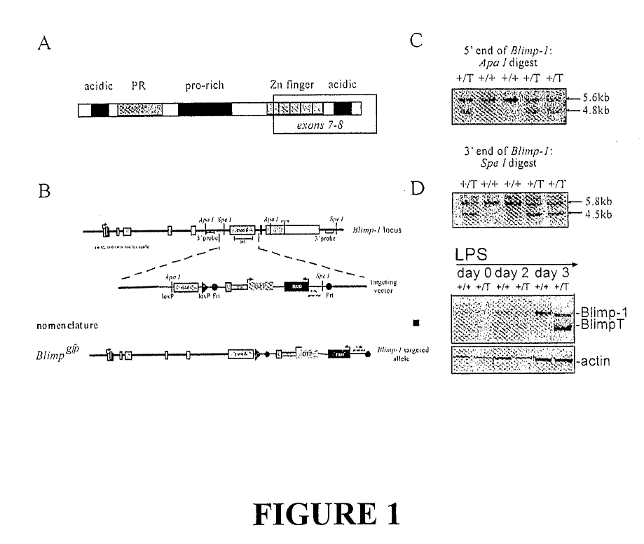

A Blimp-1 targeting construct was produced in which, inserted into the intron 3′ to exon 6, is an eGFP expression cassette consisting of a splice acceptor, stop codons in all three reading frames, an internal ribosome entry site (IRES), the cDNA encoding eGFP, and the SV40 polyadenylation signal to terminate transcription. Also inserted into the intron is a PGK-Neo^{r} gene to allow for the selection of embryonic stem (ES) cells with an integrated targeting vector. The eGFP and Neo^{r} cassettes are flanked by Frt sites to allow flp recombinase-mediated deletion of the inserted DNA. C57BL/6 ES cells were electroporated with the Blimp-1 targeting construct, resistant clones selected by G418 resistance and screened by Southern hybridisation to 5′ and 3′ genomic DNA probes (FIG. 1C). Four correctly targeted clones carrying the Blimp^{gfp} allele (FIG. 1C) were identified from 300 screened colonies, These were injected into BALB/c blastocysts to obtain chimeric founders. These chimeras have been bred, and germ-line transmission has been achieved with one clone (4F3).

### EXAMPLE 2

### A GFP Reporter that Allows the Description of the Expression Pattern of Blimp-1

Blimp-1 was initially reported to be expressed solely in B-lymphocytes that have been induced to undergo ASC differentiation (Turner et al., (supra)). However, subsequent studies have revealed a broader expression pattern of Blimp-1 during embryogenesis (Chang et al., Mech Dev 117:305, 2002) and in myeloid cells (Chang et al., (supra), 2000). The Blimp^{gfp} allele permits a fuller definition of the expression pattern of Blimp-1, both within the haematopoietic lineage and more broadly in the organism. The targeting strategy outlined above results in a Blimp^{gfp} allele that expresses GFP from a bicistronic mRNA under the control of the endogenous Blimp-1 regulatory elements and is thus predicted to recapitulate the full Blimp-1 expression pattern. In addition this strategy interrupts the Blimp-1 mRNA transcript to produce a truncated version of the Blimp-1 protein (exons 1-6) that lacks the Zinc finger domains containing the DNA binding motif. In agreement with this, Western blotting of Blimp^{gfp/+} B cells induced to differentiate with LPS in vitro demonstrated both the wild type and truncated Blimp-1 protein bands (FIG. 1D). By monitoring GFP expression in live cells and Blimp-1 protein in fixed tissue, the gene activity and differentiation fate of B-lymphocytes in vivo and in vitro at the single cell level can be monitored.

### EXAMPLE 3

### In Vivo Expression of Blimp-1 in ASC

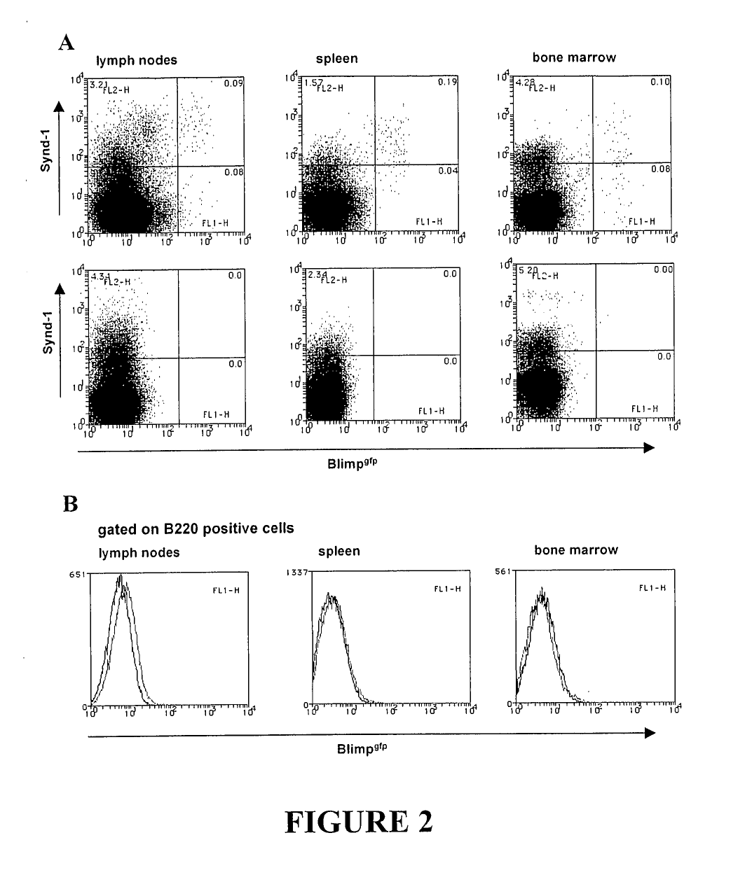

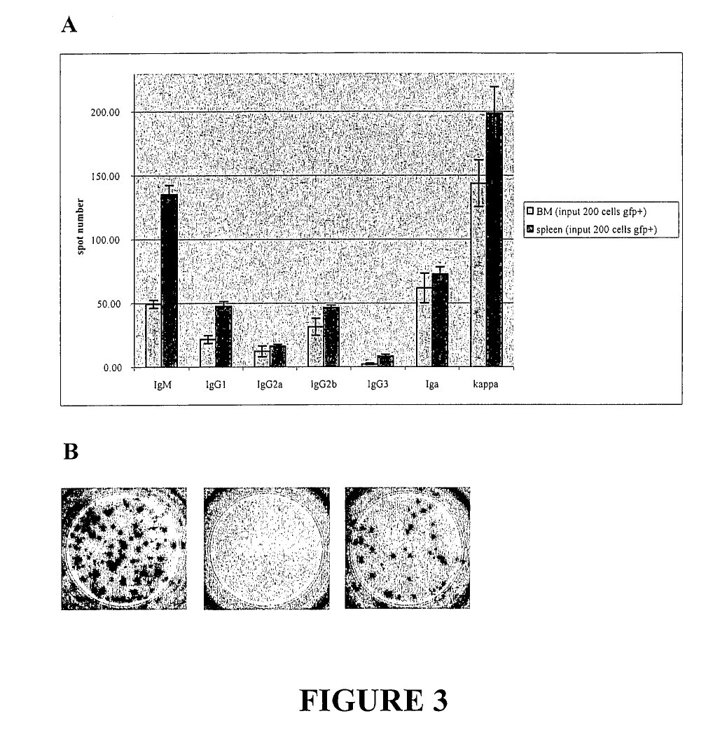

Examination of lymphoid tissues in Blimp^{gfp/+} mice demonstrated a small population of high Blimp-1 is expressing cells in the bone marrow (0.1-0.2%), spleen (0.4-0.6%) and lymph node (0.1%) (FIG. 2). Further, phenotypic analysis of the GFP⁺ cells indicated that they represented the previously defined Synd-1 high/B220 low ASC population as well as a previously poorly characterized Synd-1 low to negative phenotype (FIG. 2, (Underhill et al., (supra))). To confirm that these cells were ASC, GFP⁺ cells from Blimp^{gfp/+} bone marrow and spleen were sorted and subjected to ELISpot analysis for Ig production. As can be seen in FIG. 3, 75-100% of cells were Ig secreting cells (representative of 3 independent experiments). Moreover sorting of the GFP negative fraction, revealed a frequency of ASC of 0.001% (<1 per 100,000 cells), whereas the frequency of these cells in unsorted bone marrow was between 0.05-0.09% (50-90 per 100,000). Therefore the isolation of Blimp^{gfp} expressing ASC gives an enrichment of 100,000 fold over unsorted cells and provides a virtually definitive method to isolate these rare cells. In addition all Ig isotypes were represented in the GFP⁺ ASC populations (FIG. 3).

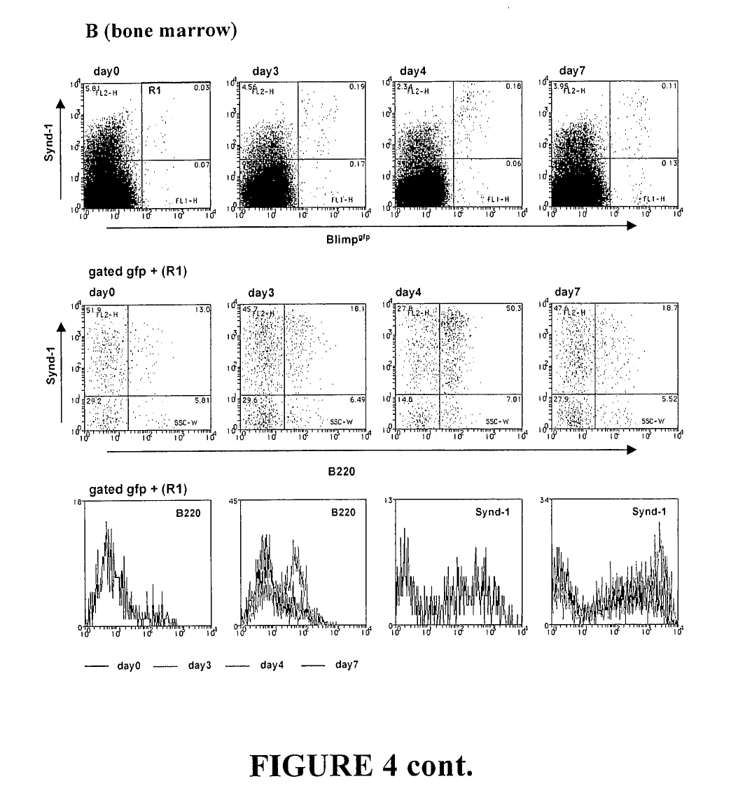

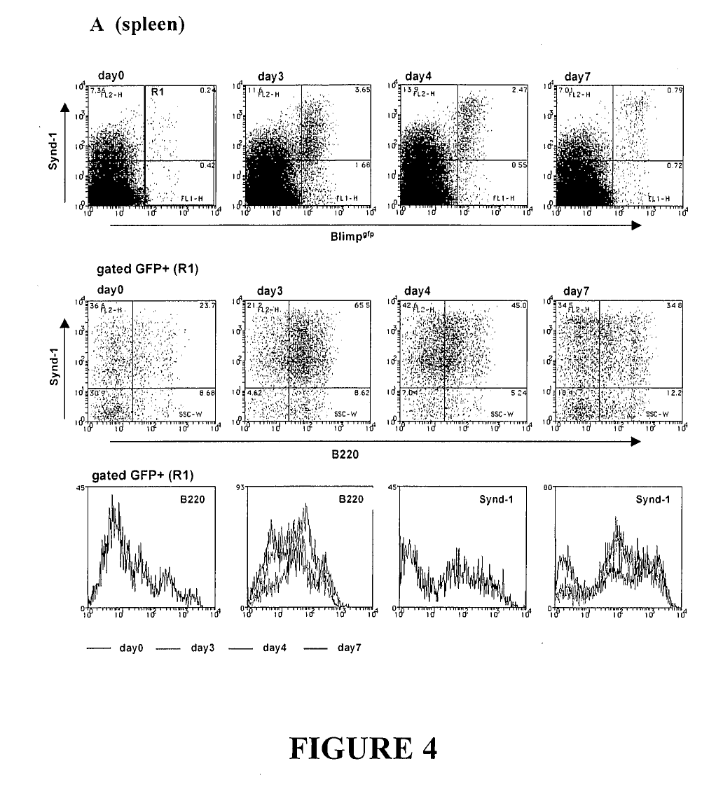

To further examine the production of ASC in Blimp^{gfp/+} mice using the GFP reporter mice were injected with 2 ug lipopolysaccharide (LPS) intravenously and analysed for GFP expression 1-7 days post-injection (LPS injection results in the polyclonal activation of mature B cells). LPS injection resulted in a dramatic increase in the numbers of splenic GFP⁺ cells peaking at day 3 post-injection (˜5% of total cells) before declining to steady state levels around day 7 (FIG. 4A). Analysis of gated GFP⁺ cells indicated that ASC differentiation occurred in a synchronous wave with the appearance of Synd-1⁺/Bp220⁺ cells followed by Synd-1⁺/B220⁻ and finally a fraction of ASC become Synd-1⁻/B220⁻. This differentiation is also mirrored in the bone marrow where GFP⁺ cells appear at day 4 post-injection as Synd-1⁺/B220⁺ cells that rapidly generate the Synd-1^{+/-}/B220⁻ steady state populations (FIG. 4B). In summary, LPS treatment induces a wave of ASC differentiation that can be for the first time phenotypically defined using the Blimp^{gfp/+} mouse stain.

### EXAMPLE 4

### Expression of Blimp-1 in ASC Derived In Vitro

A methodology was developed to quantitatively analyse the parameters affecting the commitment to and progression through the ASC lineage in vitro. This system involves the isolation of small resting B cells that are purified by Percoll gradient centrifugation and magnetic bead enrichment and cultured in the presence of a variety of stimuli that induce B cell proliferation and differentiation to ASC. These conditions include mimicking a T-dependent response using IL-4 and anti-CD40 or a T-independent reaction using LPS. In addition IL-5 can be titrated into these cultures to accelerate the rate of differentiation and anti-IgD (1.19) crosslinking carried out to activate an antigen specific response. Cultures were assayed on days 1-5 by flow cytometry to measure the frequency of Blimp^{gfp} and Synd-1⁺ expressing cells. The number of ASC in the culture was determined by ELIspot.

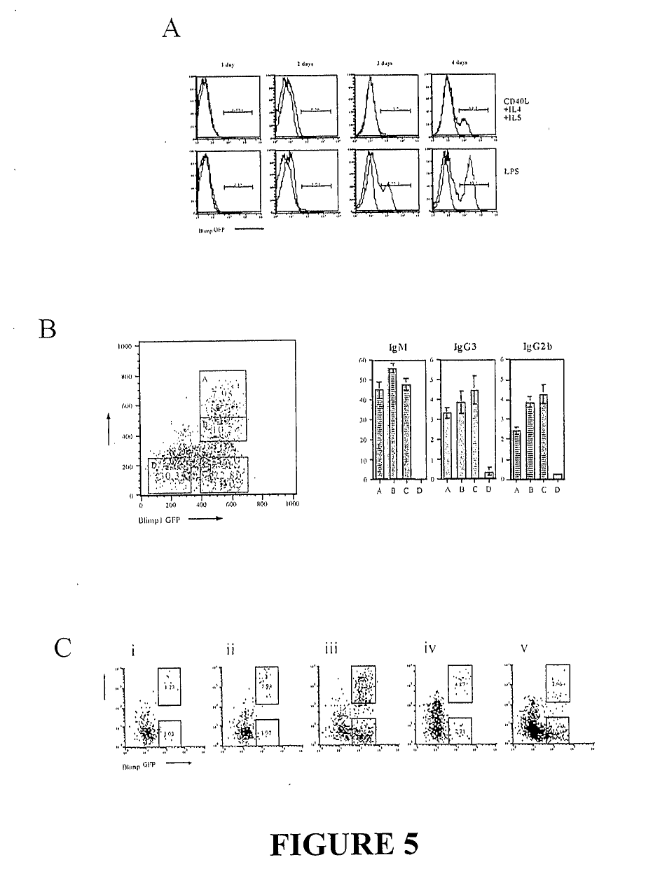

Analysis of the time course of Blimp^{gfp} induction using CD40L/IL-4/IL-5 or LPS (FIG. 5A) indicated that the first GFP⁺ cells are observed in LPS cultures after 2 days. Thereafter, the numbers of positive cells increases until a peak at day 4 of approximately 50% GFP⁺ cells. In contrast CD40L/IL-4/IL-5 treatment results in a delayed induction of fewer GFP expressing cells. Interestingly, whereas the majority of CD40L/IL-4/IL-5 induced GFP expressing cells are also Synd-1⁺, LPS induces both Synd-1⁺ and Synd-1⁻ GFP expressing cells (FIG. 5B). To determine if all the GFP⁺ cells were actually ASC four fractions from LPS treated Blimp^{gfp} B cells were sorted (FIG. 5C). ELIspot assays for IgM, IgG3 and IgG2b clearly show that all the ASC in the cultures are represented by the GFP⁺ fractions (A, B, C) and is not correlated with the levels of Synd-1 expression. Moreover, the frequency of ASC did not vary between the GFP⁺ populations (FIG. 5C). Thus these data clearly show that whilst Blimp^{gfp} is a marker of the ASC fate, Sand-1⁺ is only indicative of a sub-population of the ASC activity in vitro as it is in vivo (FIG. 2). The regulation of Blimp^{gfp} and Sand-1 expression was examined using the variety of stimuli outlined above. Interestingly, the frequency of GFP⁺/Synd-1⁺ and GFP⁺/Synd-1⁻ ASC formation can be modulated by different stimuli as outlined in FIG. 5C. Thus, following Blimp^{gfp} expression provides a simple and definitive methodology to identify the modulators of ASC induction in vitro and in vivo.

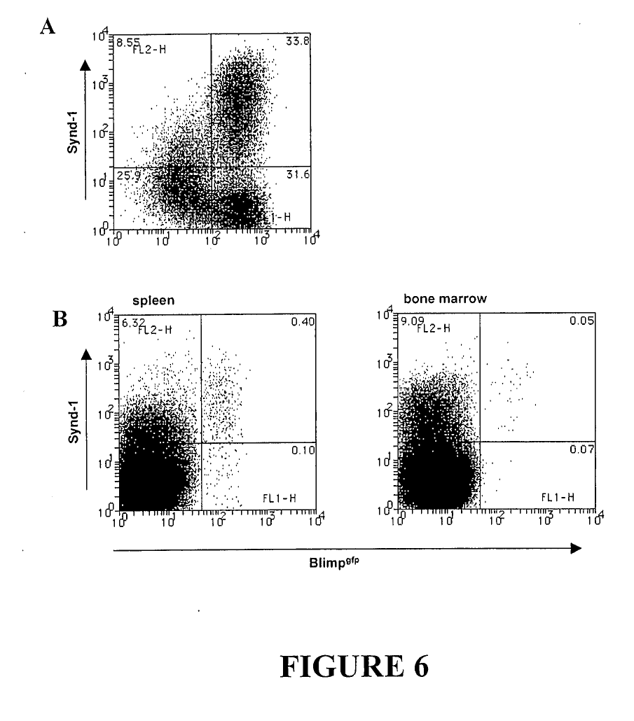

Finally, a transplantation model was developed to demonstrate that in vitro derived Blimp^{gfp} positive ASC can be detected in the bone marrow or spleen of non-irradiated hosts 7 days after intravenous injection (FIG. 6). Therefore, the Blimp^{gfp} allele provides a method to examine the effects of in vitro treatments of ASC on their survival, migration and functional properties in vivo.

### EXAMPLE 5

### Blimp-1 is Required for Embryogenesis

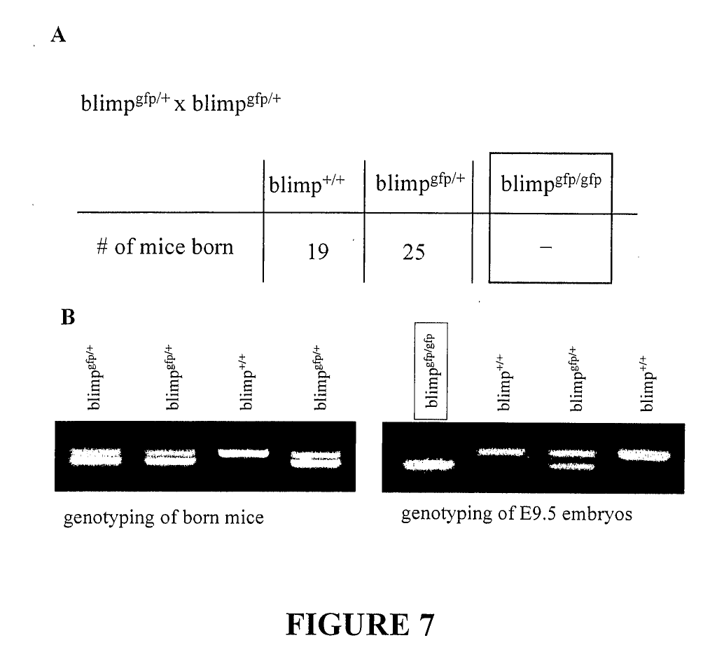

To produce homozygous Blimp^{gfp/gfp} animals Blimp^{gfp/+} individuals were intercrossed. Offspring from these crosses were genotyped at day 21 post-birth using Blimp-1 wild type and Blimp^{gfp} specific PCR primers. Whereas Blimp^{gfp/+} mice were alive and healthy, no Blimp^{gfp/gfp} individuals were identified indicating that Blimp-1 deficiency results in embryonic or early post-partum lethality (FIG. 7). To examine more closely the stage at which Blimp^{gfp/gfp} animals die, embryos produced from timed matings of Blimp^{gfp/+} mice were examiner. These data indicate that Blimp^{gfp/gfp} embryos are alive as late as embryonic stage E15.5. However, no viable older individuals have been documented. Blimp-1 is known to be widely expressed during embryogenesis, a finding that is supported by the analysis using the Blimp^{gfp} mouse.

### EXAMPLE 6

### Blimp-1 is Essential for Antibody Production

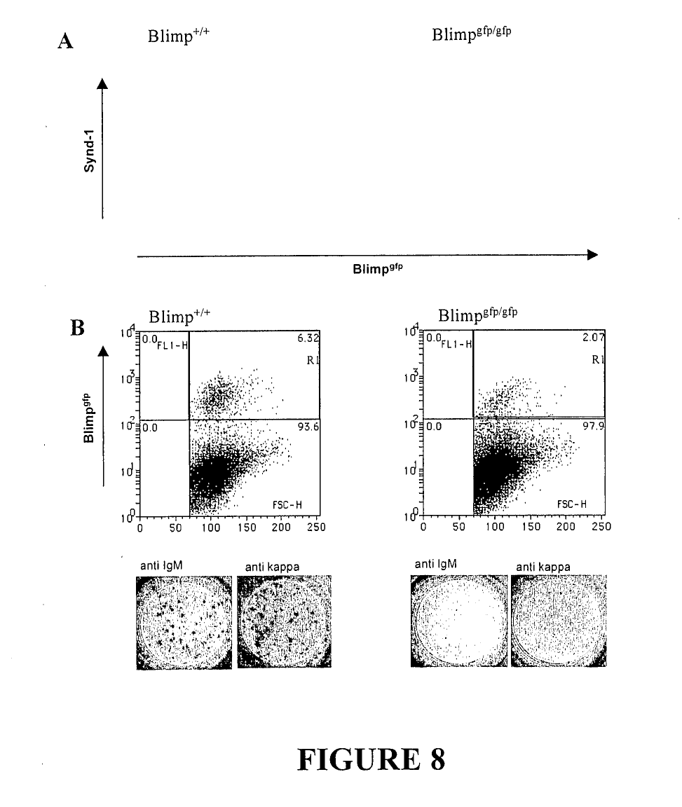

To circumvent the embryonic lethality of Blimp^{gfp/gfp} animals, and examine directly the importance of Blimp-1 in antibody production fetal liver stem cell reconstitution of lethally irradiated syngenic mice was used to produce adult mice that lack functional a functional Blimp-1 protein throughout the haematopoietic system. These Blimp^{gfp/gfp} chimeric animals are healthy and contain relatively normal numbers of all the haematological lineages examined. In vitro analysis of the ASC population in these mice following stimulation with either LPS or CD40L/IL-4 and IL-5 revealed that the presence of GFP⁺ Blimp deficient cells that were predominantly synd-1⁺ (FIG. 5A). Importantly, these cells failed to secrete antibody as assessed by ELIspot assay (FIG. 8B). Therefore, the Blimp^{gfp} the mouse model described here not only provides a definitive tool to isolate ASC but enables the identification of the population of Blimp-1 expressing cells from homozygous mutant Blimp^{gfp/gfp} splenocytes, thereby greatly facilitating the analysis of the mechanism underlying the phenotype of Blimp-1 deficiency.

### EXAMPLE 7

### Expression of Blimp-1 in Other Haematopoietic Lineages

The Blimp^{gfp} reporter system has also enabled for the first time define the expression pattern of Blimp-1 in haematopoiesis. As stated above analysis of the lymphoid organs of Blimp^{gfp} mice revealed that the GFP high producing populations are almost exclusively ASC. However, lower level GFP producing cells were also apparent.

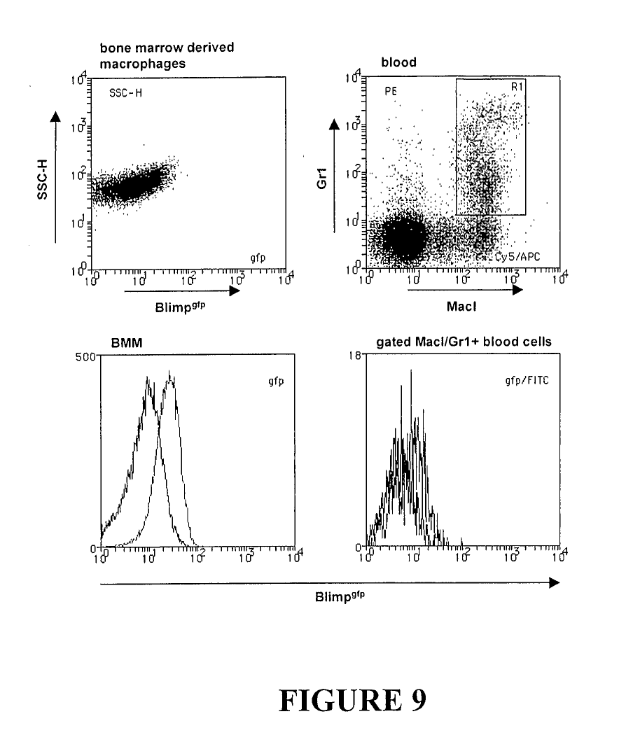

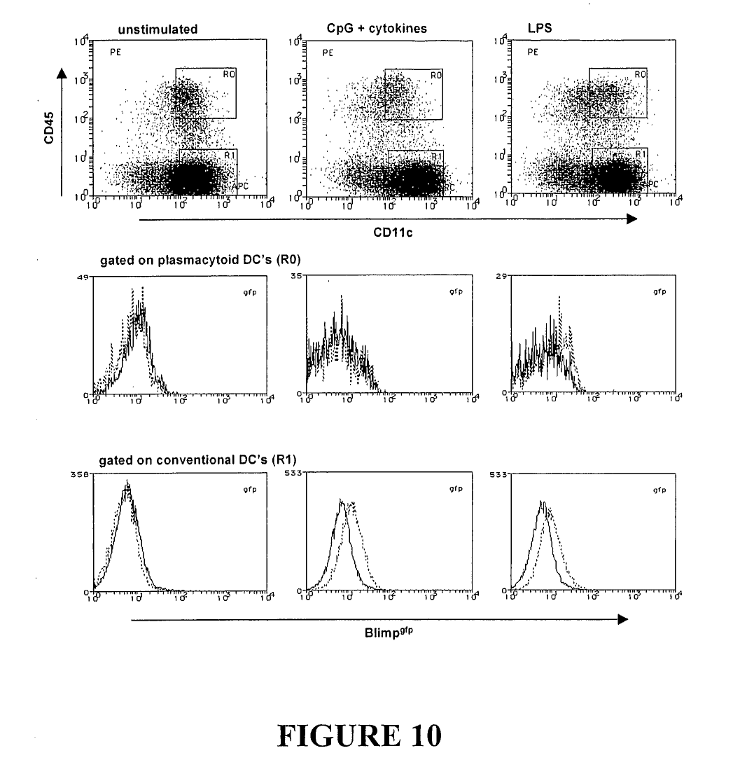

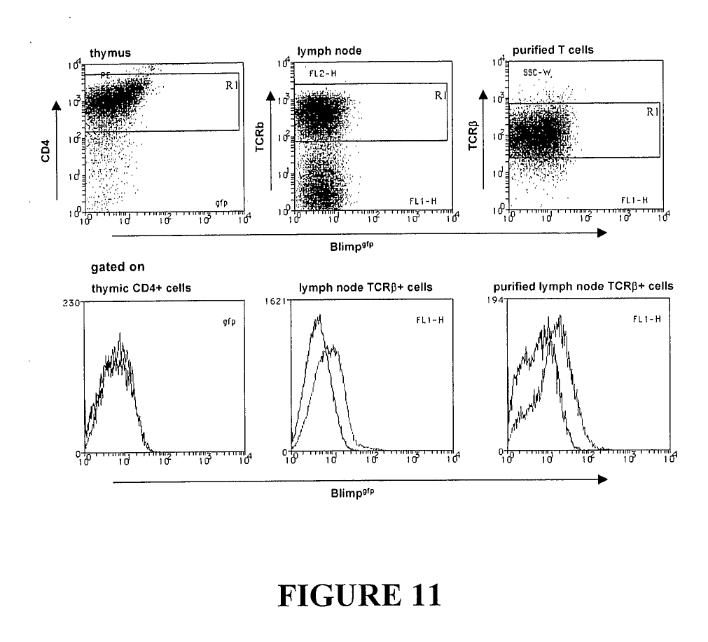

Blimp-1 has been reported to be expressed by human and mouse macrophages and granulocytes. Flow cytometric analysis of blood monocytic cells and and bone marrow derived macrophages cultures in the presence of MCSF-1 revealed clear Blimp^{gfp} expression in these cell types (FIG. 9). However, no GFP fluorescence was observed in granulocytes. In vivo isolated dendritic cells in contrast lack Blimp-1 mRNA expression. Similarly, plasmacytoid and conventional dendritic cells derived from the culture of bone marrow cells with flt3L lack Blimp^{gfp} fluorescence. However, the ex vivo activation of sorted dendritic cells or the in vitro activation of the flt3L cultures by CpG DNA results in Blimp-1 expression predominantly conventional dendritic cells (FIG. 10).

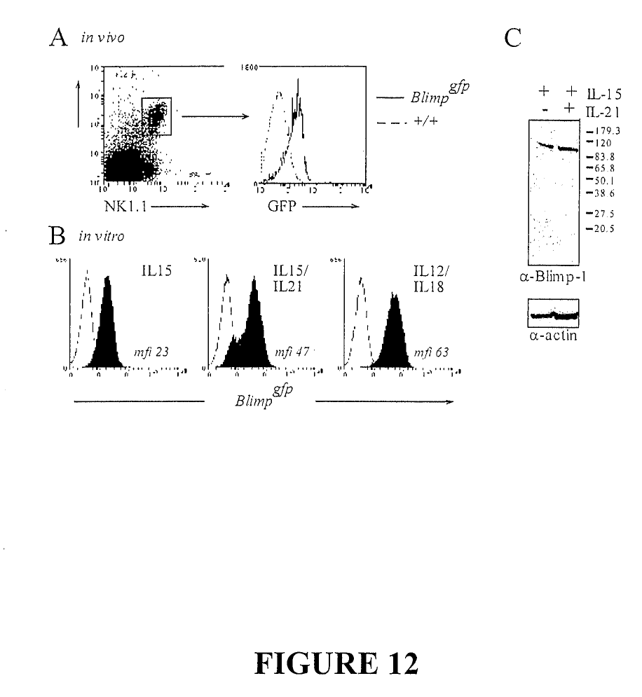

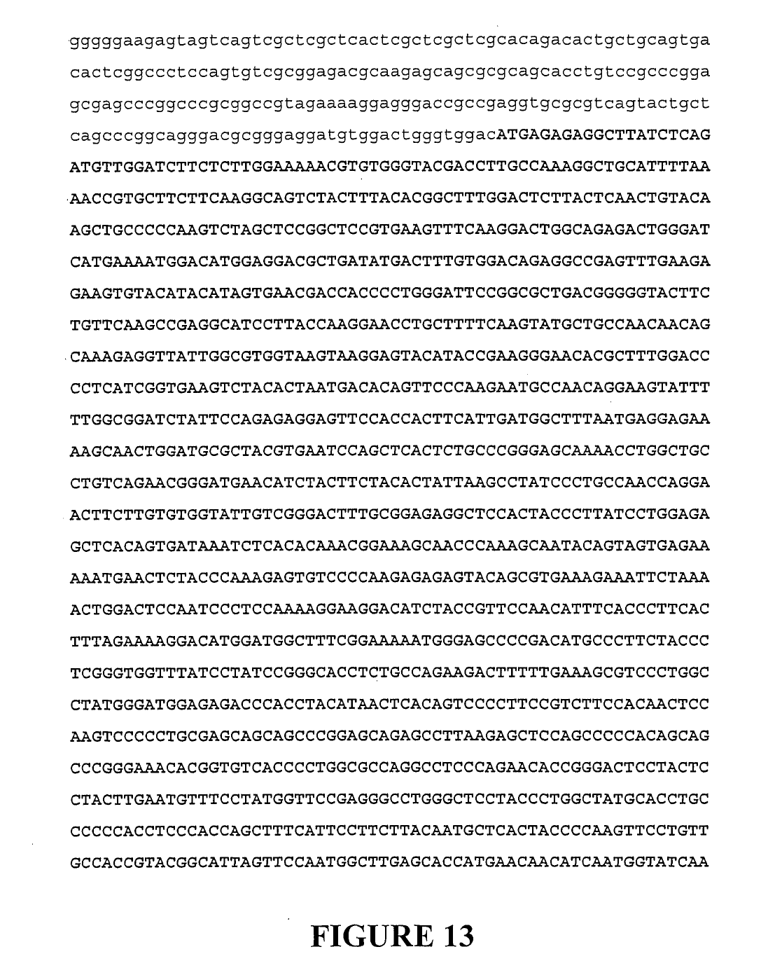

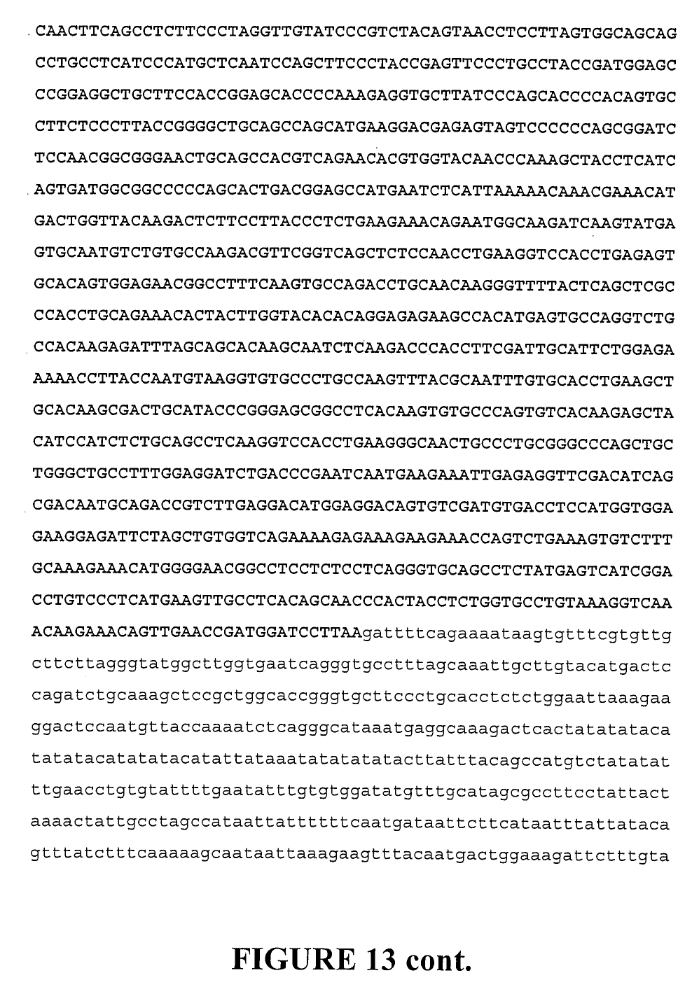

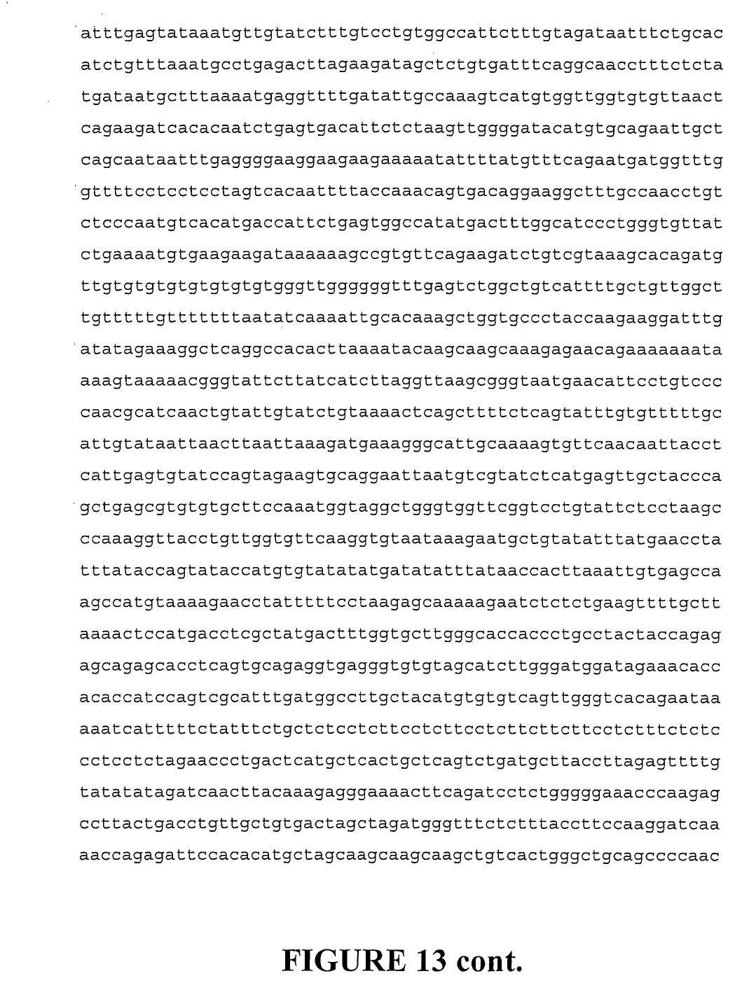

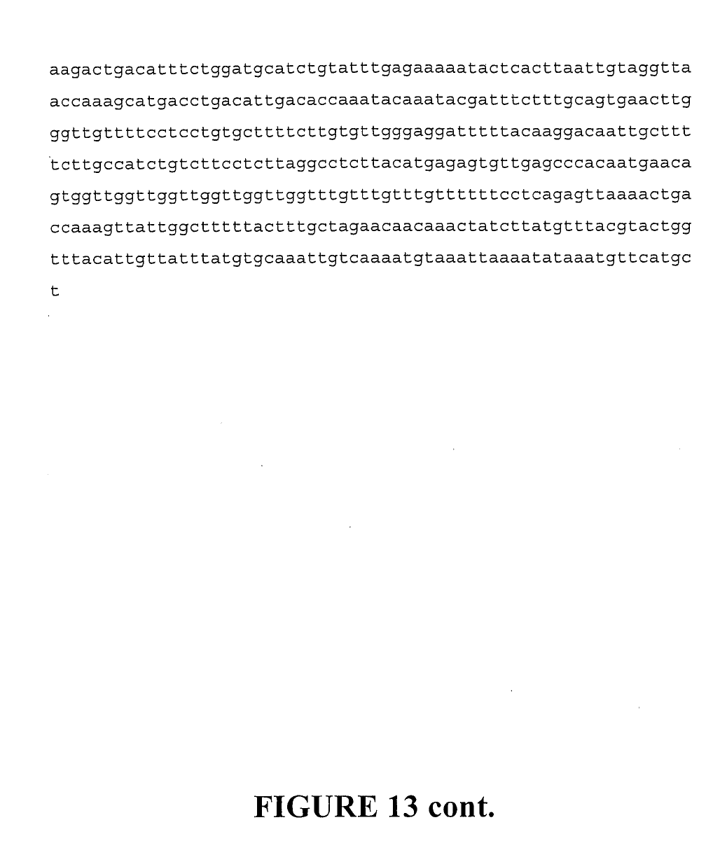

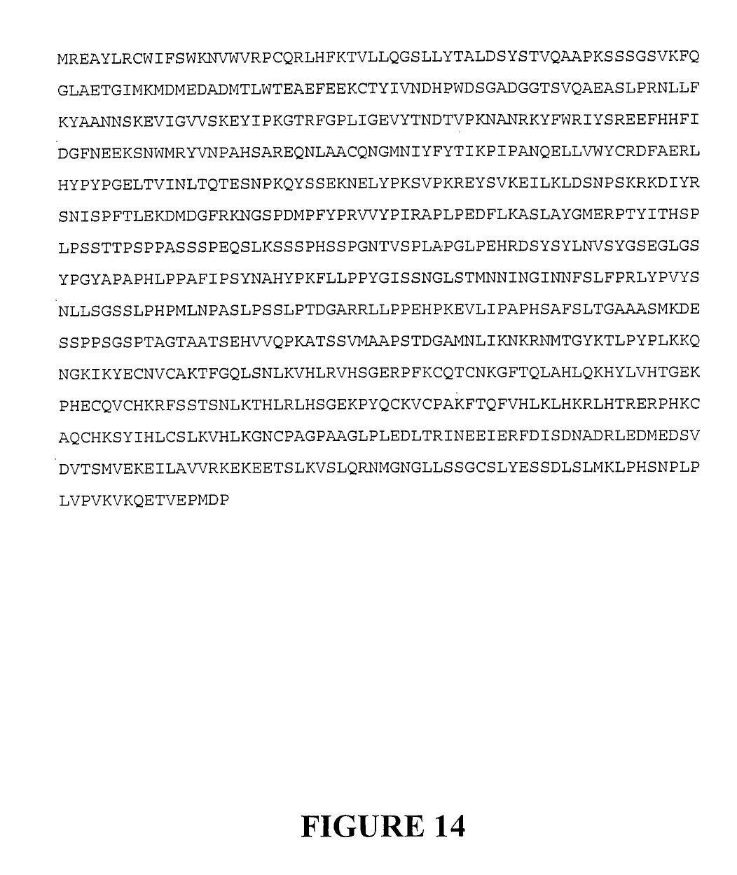

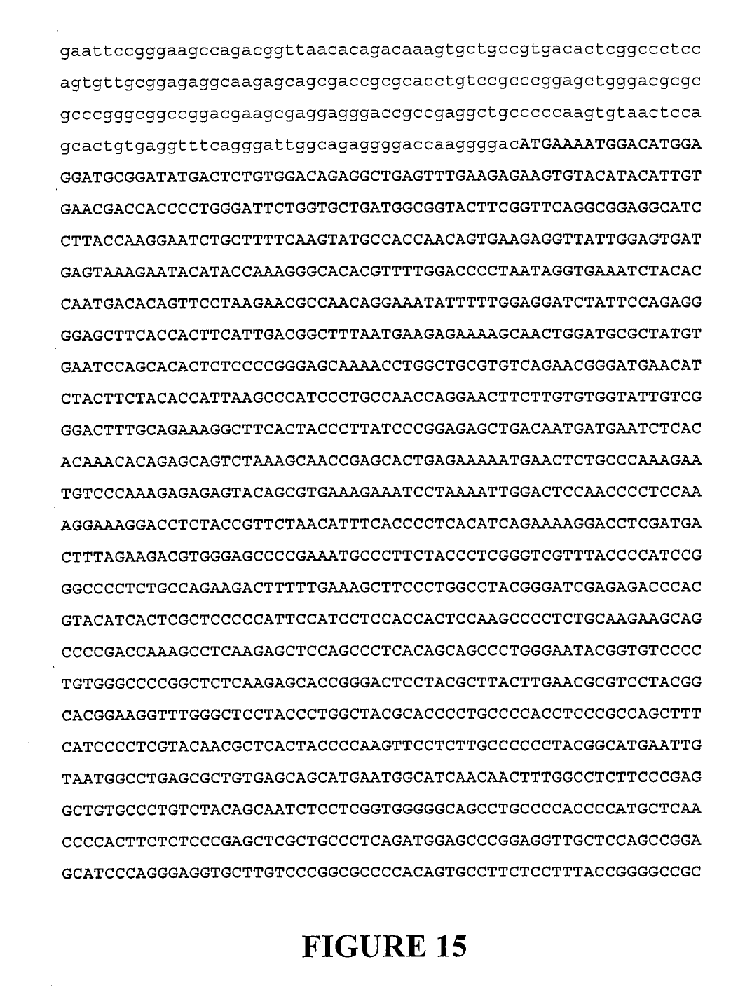

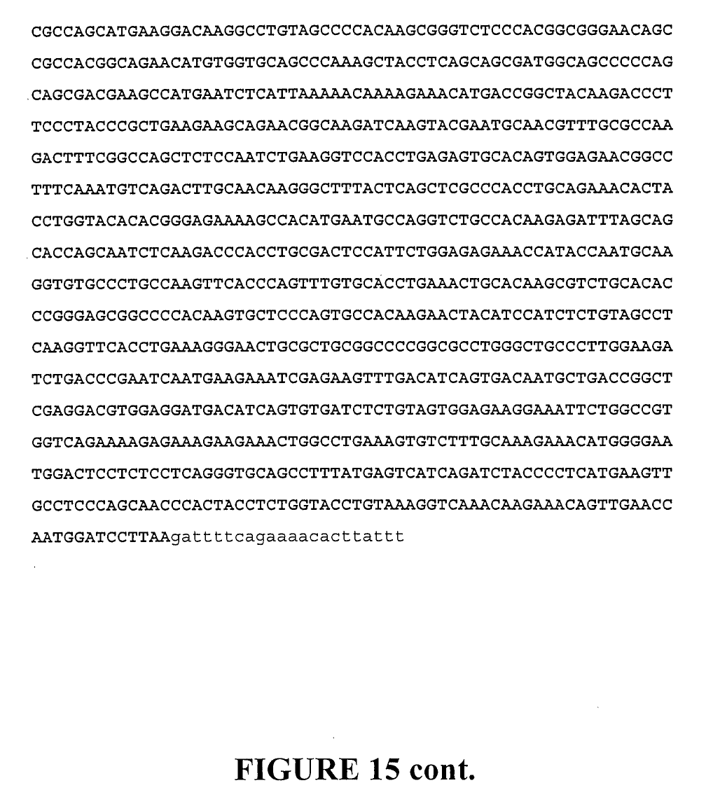

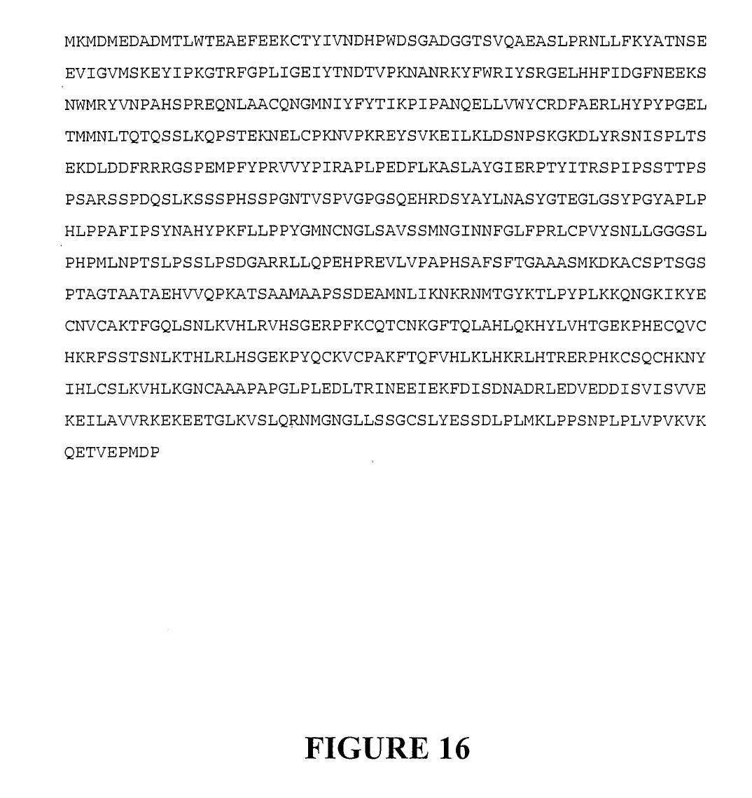

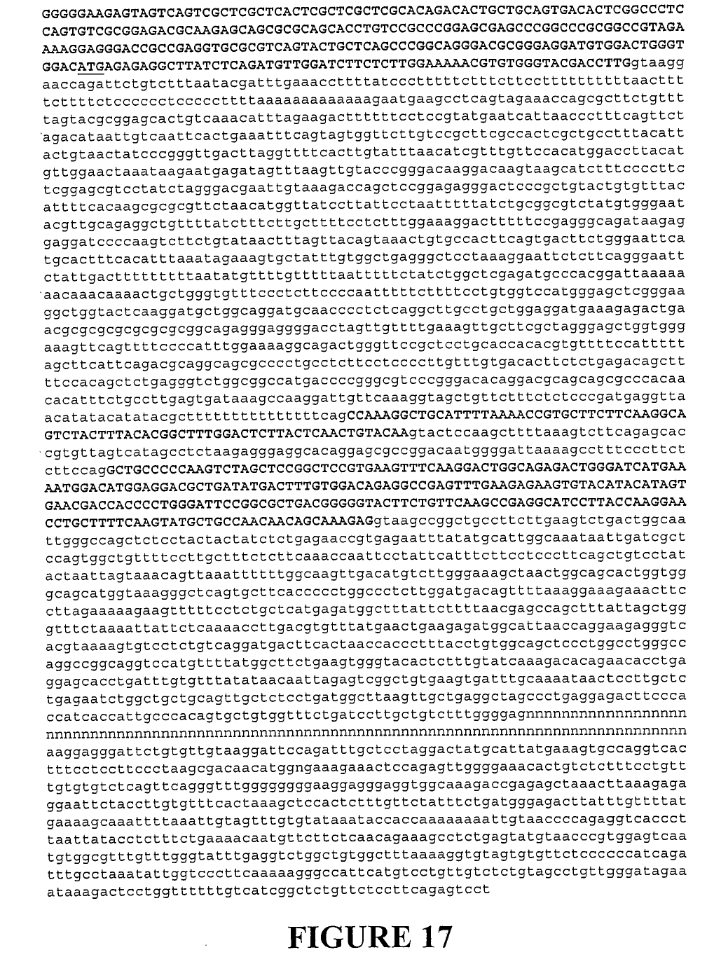

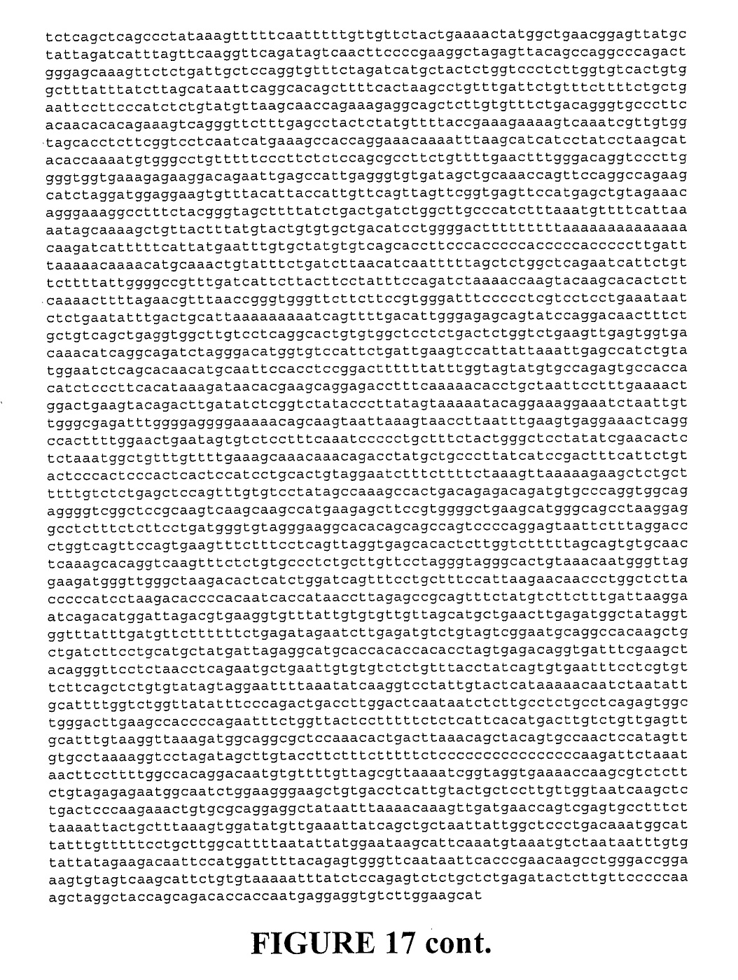

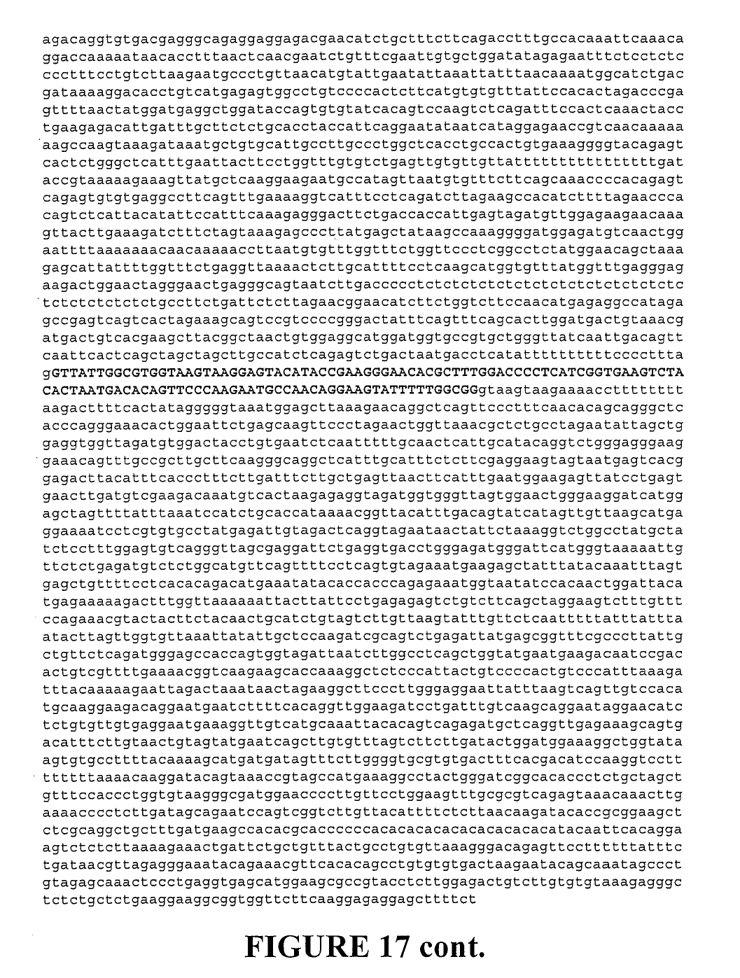

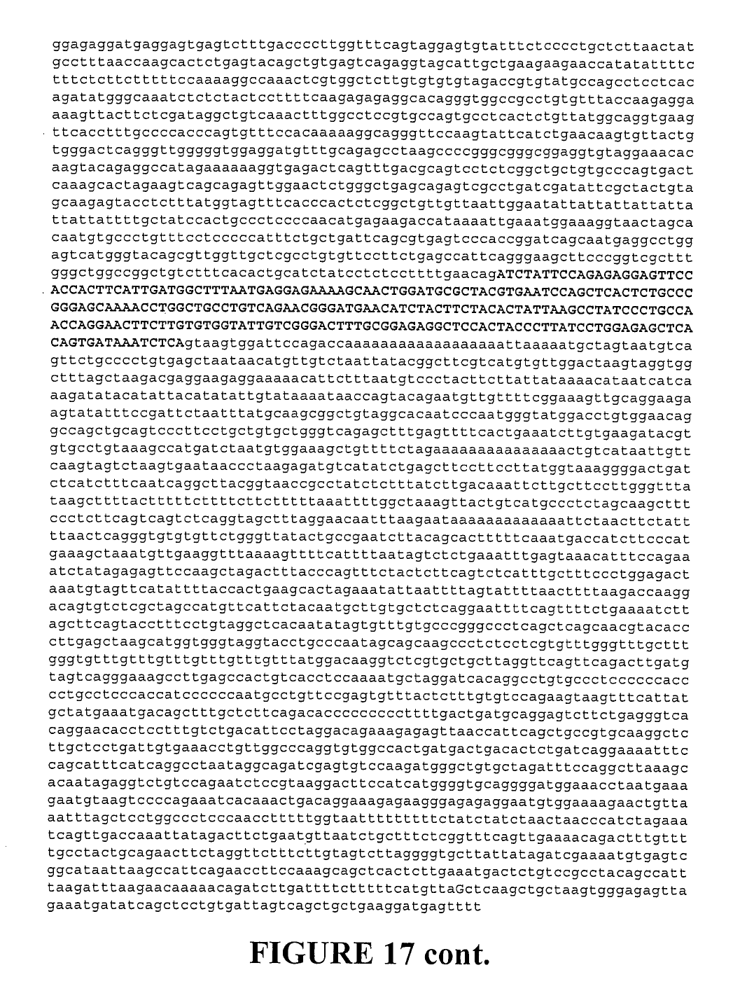

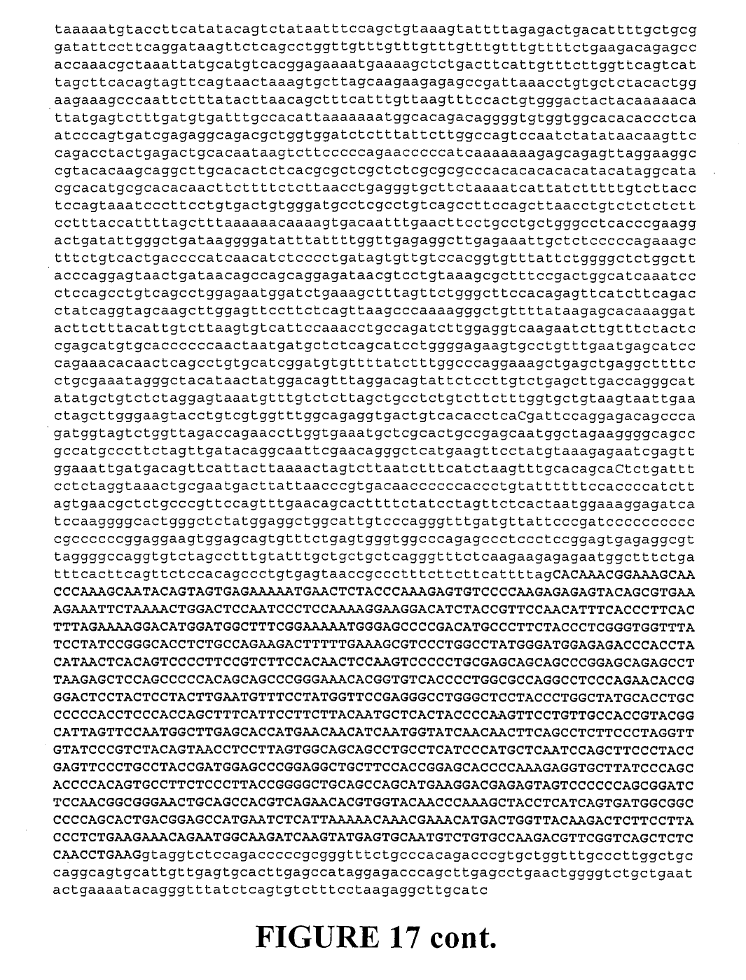

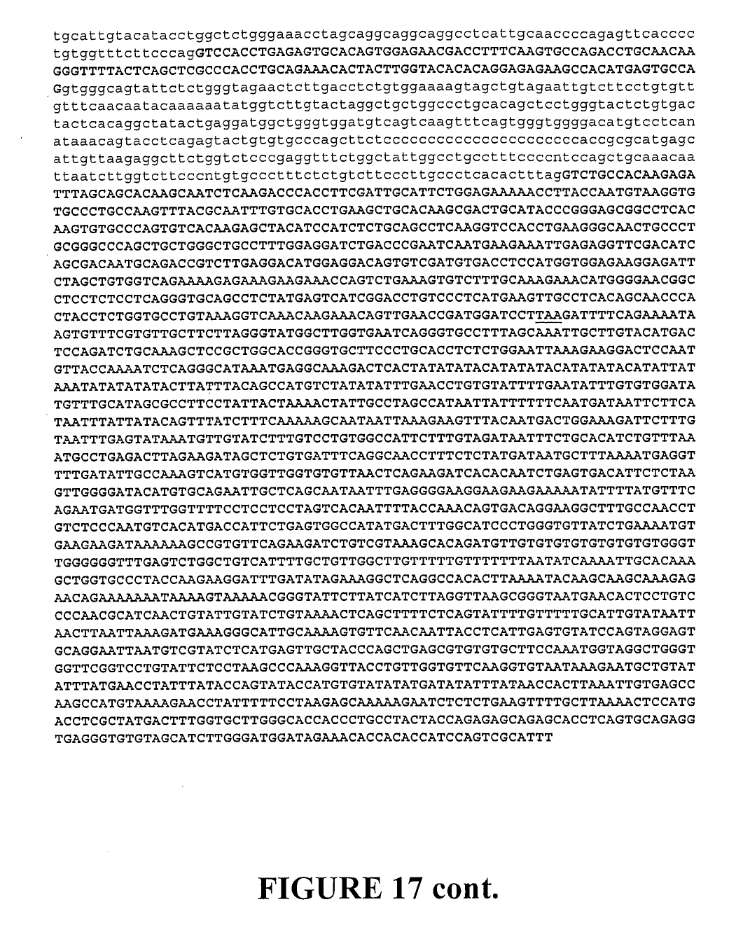

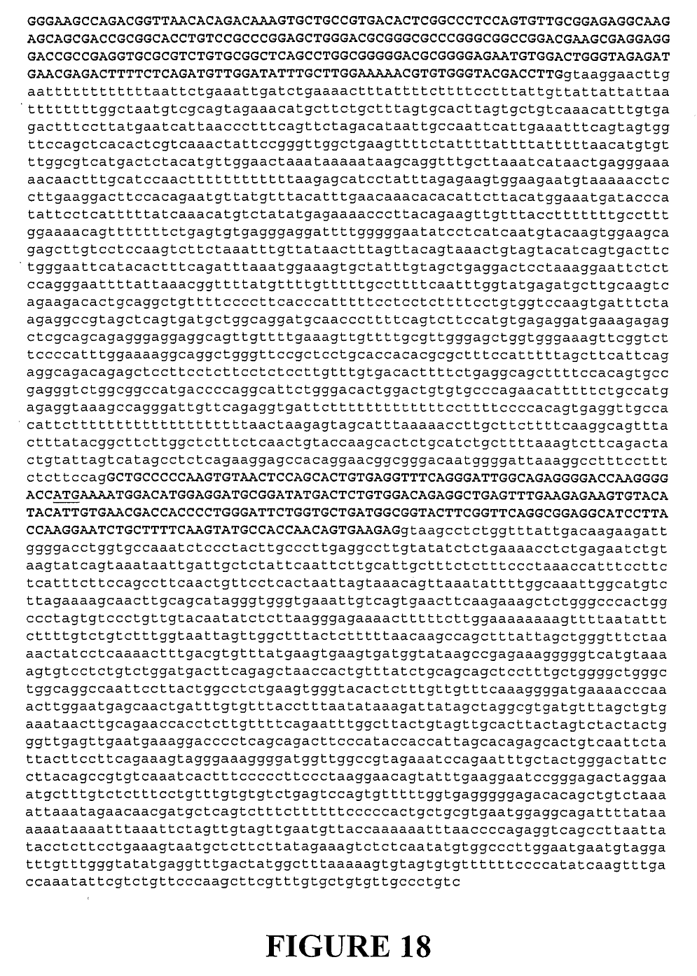

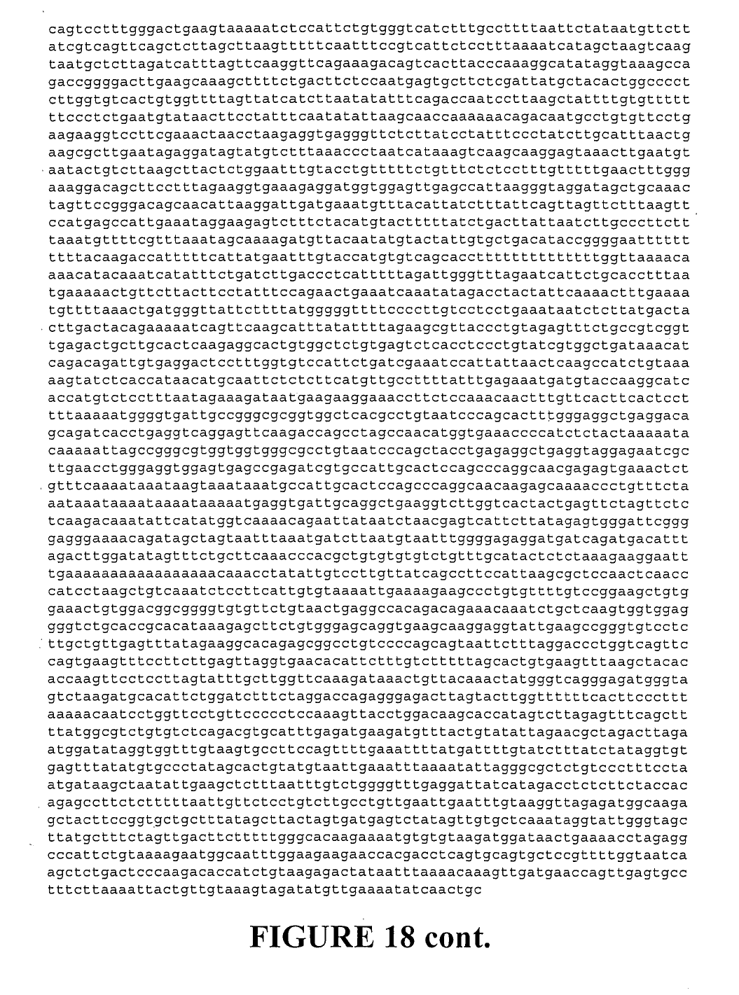

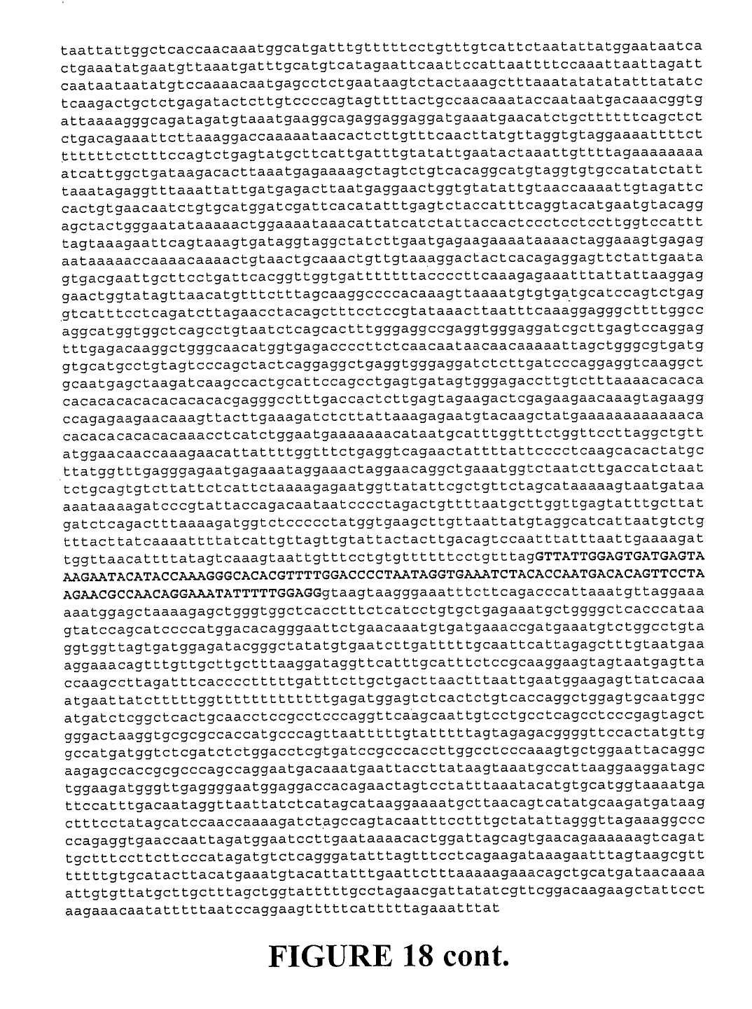

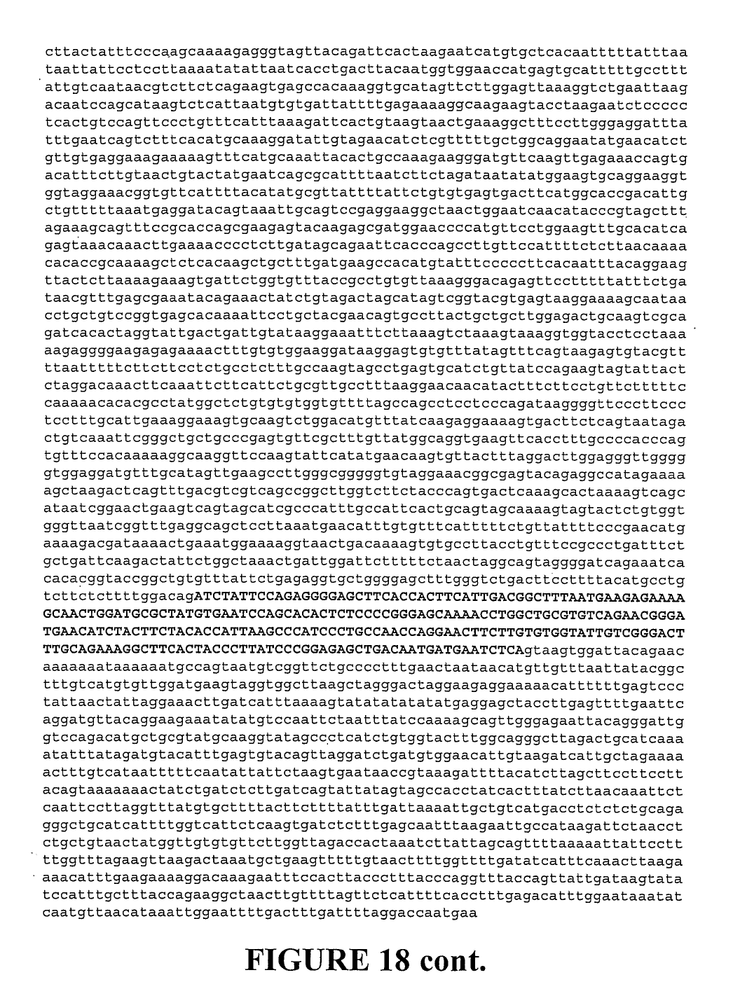

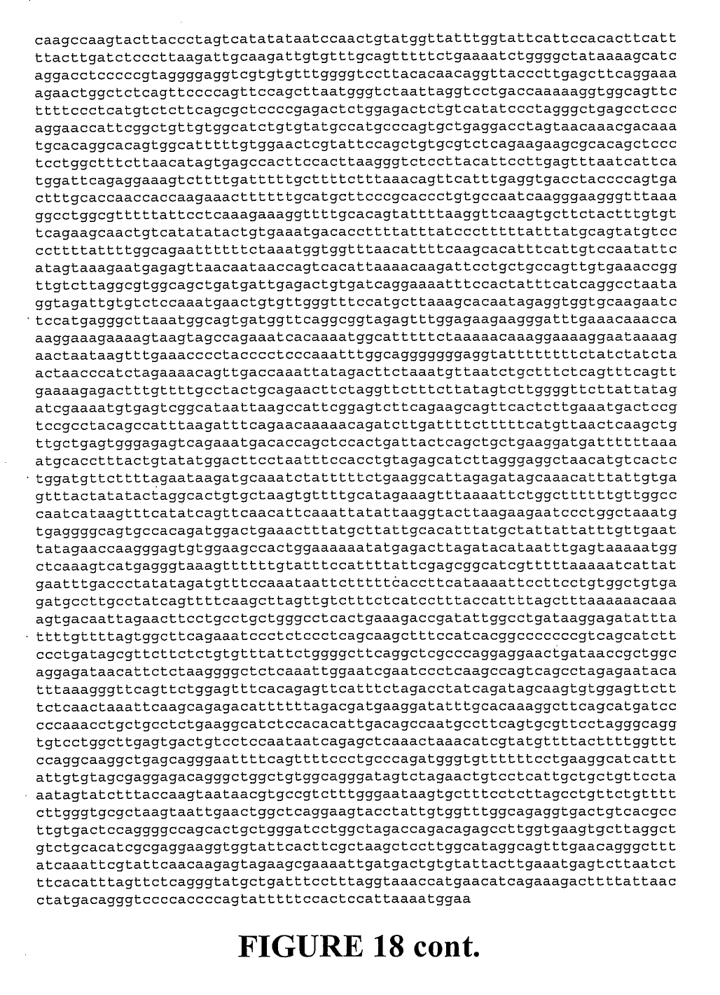

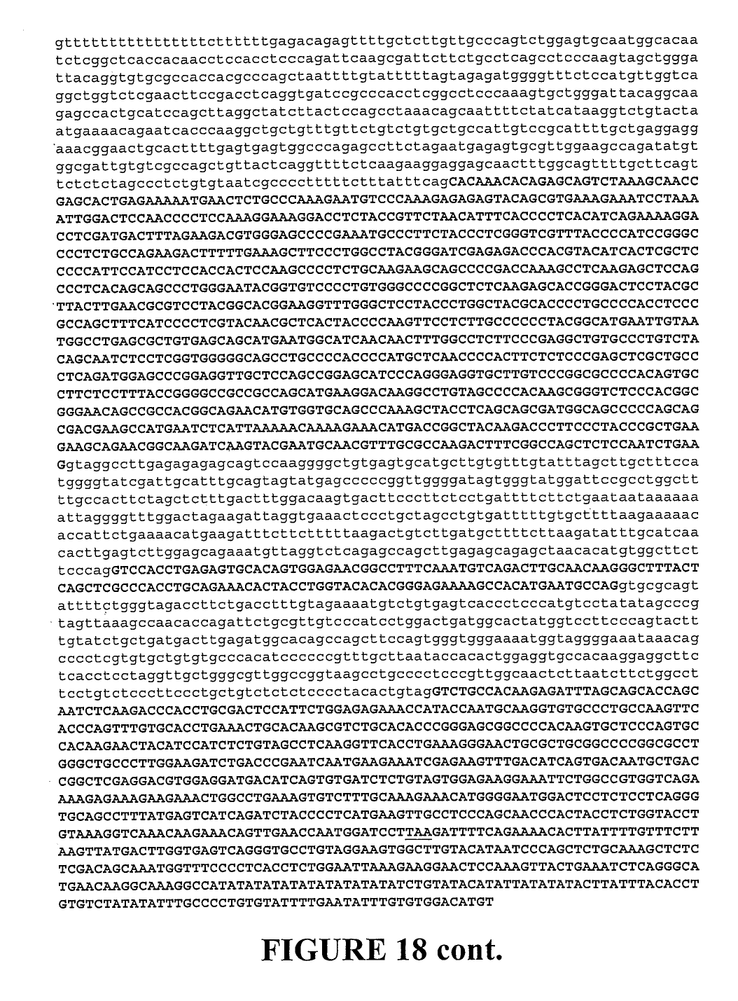

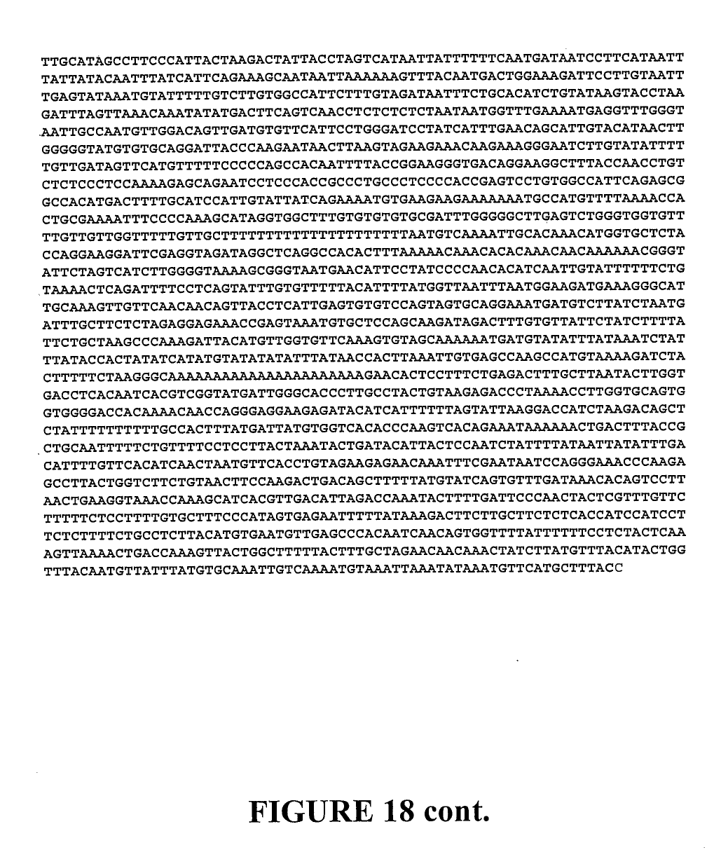

Analysis of thymus and resting spleen from Blimp^{gfp/+} mice demonstrated that Blimp-1 is not expressed during T cell development. However, a small population of Blimp^{gfp} expressing T cells were present in lymph nodes. As these cells could represent the small population of activated T cells present we have stimulated lymph node T cells in vitro with an anti-CD3 monoclonal antibody in the presence or absence of concanavalin A, conditions known to strongly activate T cells. In support of the in vivo analysis, in vitro activated T cells expressed Blimp^{gfp} (FIG. 12C).

Examination of the NK lineage in Blimp^{gfp} mice revealed that unlike the other haematopoietic lineages examined NK cells constitutively express Blimp-1. NK cells were identified from blood, spleen and bone marrow as NK1.1⁺/CD122⁺ cells and demonstrated to be uniformly GFP⁺(FIG. 12A). This expression was maintained in vitro as mature NK cells cultured in the presence of IL-15 are GFP⁺ and can be further induced by cytokines such as IL-21 or IL-12/IL-18 that induce NK cell terminal differentiation (FIG. 12B). The expression of Blimp-1 in NK cells was also confirmed by Western blotting with a Blimp-1 specific monoclonal antibody.

In summary, the Blimp^{gfp} reporter mouse has revealed that Blimp-1 is induced in the late stages of a variety of haematopoietic lineages thereby providing a method of identifying the regulators of the maturation of these cell types. Importantly, the relatively lower production levels of GFP in non-B lymphoid cell types does not interfere with the isolation of homogenous populations of ASC.

### EXAMPLE 8

### Examining the Role of Blimp-1 in Cancer Using the Blimp^{gfp} Mouse

In addition to its utility in examining ASC differentiation, the Blimp^{gfp} reporter mouse can be used to examine the malignant transformation of this cell type. Tumors of ASC, designated plasmacytomas in mice and multiple myeloma in humans, are specifically and frequently elicited in Eμ-v-abl transgenic mice (Rosenbaum et al., (supra)), which express the v-abl oncogene in the B cell lineage, under the control of the IgH intronic enhancer. These mice were crossed with Blimp^{gfp} mutant mice, to determine the affect of loss of one or both copies of the Blimp-1 gene on latency and incidence of tumors. Two outcomes are envisaged: Blimp-1, by inducing the plasma cell differentiation program, might be required to open the window of opportunity for v-abl transformation. This transgene induces only plasmacytomas, despite expression in earlier B cells (Rosenbaum et al., (supra)). Therefore, loss of functional Blimp-1 alleles would be predicted to decrease tumor incidence or increase latency. Alternatively, as a large proportion of v-abl-induced plasmacytomas also bear a rearranged and activated c-myc gene, it may be that Myc is an essential cooperating activity in the transformation (Rosenbaum et al., (supra)). Blimp-1 is believed normally to repress c-myc expression during terminal ASC differentiation (Lin et al., Science 276:596, 1997). In this scenario, loss of functional Blimp-1 should allow continued c-myc expression, which may accelerate plasmacytoma development.

If Blimp-1 is indeed playing a role in ASC tumorogenesis, the Blimp^{gfp} reporter strain provides, therefore, a useful animal model to determine the effects of inhibiting/inducing Blimp-1 on tumor progression.

### EXAMPLE 9

### Methods for the Assessment of the Role of Blimp in Regulating Terminal Differentiation in T-Cells

Mice

Blimp^{gfp} (Kallies et al., J Exp Med 200:967-977, 2004), Rag1^{-/-}, and Rag2^{-/-} mice were maintained on a C57BL/6 background. Blimp^{gfp} genotyping and foetal liver chimeras were generated as described (Kallies et al., 2004 (supra)).

Flow Cytometry and ELISAs

Monoclonal antibodies (mAbs) against CD4 (GK1.5), CD8 (53.6.7), TCRβ (H57-597), Ly5.2 (ALI-4A2) were purified from hybridoma supernatants on Protein G-sepharose columns (Amersham Pharmacia Biotech) and conjugated to biotin (Pierce Chemical Company), allophycocyanin (APC) and phycoerythrin (PE) (ProZyme) as recommended. Biotinlyated anti-CD25 (7D4) and CD122 (Tm-β1) and PE conjugated anti-CD44 (IM7), CD62L (MEL-14) IFNγ, [symbol] IL-4 (11B11) were obtained from PharMingen. Biotinylated mAbs were revealed with Streptavidin-PE or Cy5 (Southern Biotechnologies Inc). Cells were analyzed on a LSR cytometer (BD Biosciences) and cell sorting was carried out on high-speed flow cytometers (Moflo cytomation and BD Biosciences). Intracellular staining for cytokines was carried out according to standard procedures known in the art. ELISA for IFNγ and IL-10 production was performed as described (Brady et al., J Immunol 172: 2048-2058, 2004). IL-4 ELISA used one monoclonal antibody as a capture reagent and a second monoclonal antibody for detection. ELISAs were performed in triplicate and quantified using recombinant protein standards.

Western Blotting

Total protein extracts were produced from equivalent numbers of cells and Western blotting was carried out for example as described by Rosenbauer et al., Embo J 21:211-20, 2002). Anti-Blimp-1 mAb has been previously described (Kallies et al., 2004 (supra)). Equal protein loading was confirmed using anti-Vav1.

HSV Infection

Mice were infected with 4×10⁵ Herpes Simplex Virus (HSV-1 KOS strain) diluted in 20 μl PBS. Administration was by subcutaneous injection into the hindleg between the footpad and heal. Spleen and popliteal lymph nodes of infected mice were subsequently harvested for analysis.

In Vitro Cytotoxicity

gB-specific cytotoxic T-Cell lymphoctyes (CTL) were generated by routine procedures. [Belz, 2001]. Spleens were removed from infected mice and single cells were cultured with 10⁸ 1000 Gy-irradiated gB₄₉₈₋₅₀₅-coated C57BL/6 spleen cells for 5 days. Cytotoxicity was assessed in a conventional ⁵¹Cr-release assay. The EL4 (H-2^{b}) target cells were labelled with Na⁵¹Cr for 1 h and pulsed with gB peptide 60 min, washed twice, and plated at 5,000 targets/well. They were then incubated with the effector populations for 5 h before harvesting supernatants for gamma counting. Two-fold lymphocyte dilutions were assayed in triplicate, while untreated and Triton X-100-disrupted controls were measured in quadruplicate. The percent specific lysis was calculated as 100×(⁵¹Cr release from targets with effectors−⁵¹Cr release from targets alone)/(51Cr release from targets with Triton X-100). The level of ⁵¹Cr release from targets incubated in the absence of T cells was <10% of the total Triton X-100-mediated ⁵¹Cr release.

Tetramer Staining of gB-Specific CD8⁺ T Cells

Virus-specific CD⁸⁺ T cells were identified using MHC class I tetrameric complexes [Altman, 1996 #102; Allan, 2003 #100] of the H-2K^{b} glycoprotein and peptide (SSIEFARL) derived from the glycoprotein B of herpes simplex virus (gB₄₉₈₋₅₀₅). Recombinant H-2K^{b} molecules with a birA biotinylation motif substituted for the carboxyl-terminal transmembrane domain were refolded with human β₂-microglobulin plus the viral peptide, biotinylated with birA and complexed at a 4:1 molar ratio with neutravidin-PE (Molecular Probes, Eugene, Oreg.). Lymphocytes were stained for 60 minutes at room temperature with the tetrameric complexes in PBS/BSA/azide, followed by staining with anti-CD8αAPC, washed twice, and analyzed by flow cytometry.

Histology

Organs were fixed in 10% buffered formalin, embedded in paraffin, sectioned and stained with hematoxylin/eosin.

### EXAMPLE 10

### Blimp Expression can be Induced by IL-21, is a Key Component of the Maturation of Naïve CD4⁺ and CD8⁺ T-Cells into Activated Effector Cells and is Essential for Normal Lymphocyte Homeostasis

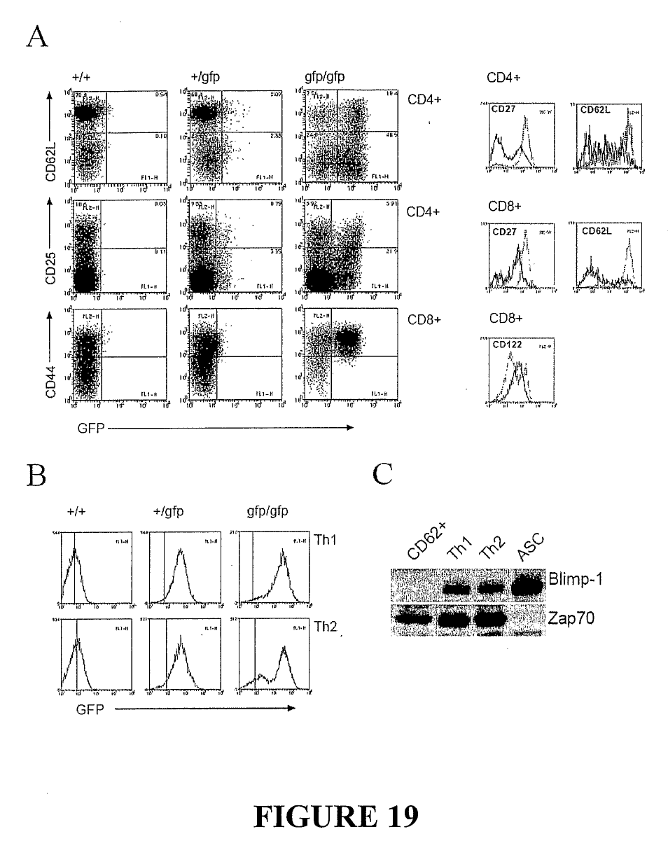

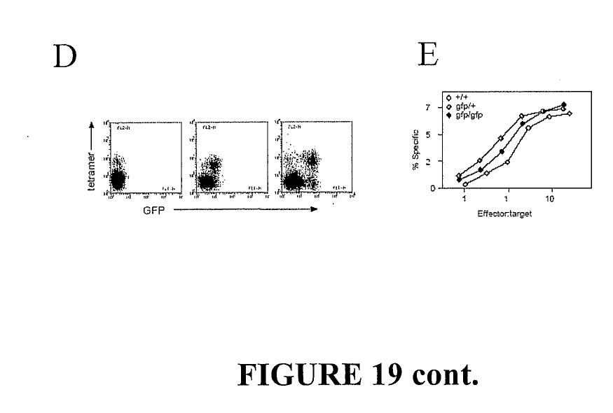

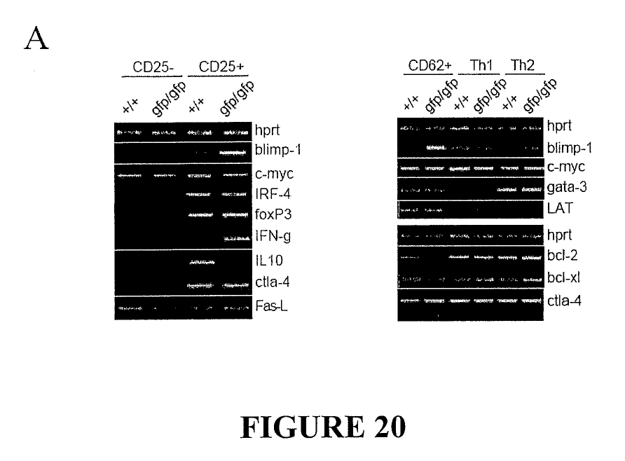

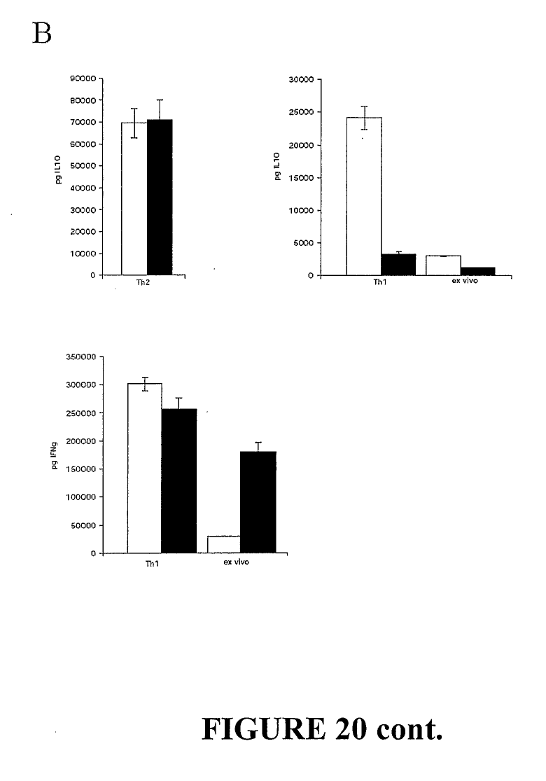

Examination of the lymphoid organs from Blimp^{gfp/+} mice revealed a high level GFP in plasma cells. Furthermore, a population of T cells expressed low-levels of GFP (FIG. 19A). Further analysis of Rag1^{-/-} mice reconstituted with Blimp^{gfp/gfp} cells, expressing no functional Blimp alleles revealed a pronounced expansion of this same population and a significant increase in GFP fluorescence (FIG. 20). More extensive flow cytometric analysis of the T cell compartment of these mice revealed no GFP fluorescence in thymocytes (data not shown) or naïve T cells (FIG. 20).

In contrast and as shown herein, Blimp-1 is expressed specifically in activated/memory type CD4⁺ (TCRβ⁺CD62L^{low}) and CD8⁺ (TCRβ⁺CD44^{high}) T cells (FIG. 20). In a more detailed FACS analysis, alternate markers have been used to determine the nature of the expanded T cell pool in Blimp-1 deficient mice. In contrast to mice reconstituted with wildtype fetal liver, Rag1^{-/-} mice reconstituted with Blimp^{gfp/gfp} cells showed a strong expansion of the CD62L low GFP positive T cell population. Further, these mice had strongly elevated numbers of CD27 low to negative T cells, an increase in CD25 positive CD4 cells as well as a lack of distinct CD122 high population. Analysis using alternate markers, such as CD69, confirmed the activated status of the GFP⁺ cells (data not shown). The increased numbers of effector CD4 T cells in Blimp^{gfp} reconstituted Rag1^{-/-} mice was confirmed by ex vivo stimulation of isolated splenic CD4 T cells, resulting in 8 to 10 times higher IFN secretion, while secretion of IL-10 was decreased.

In vitro culture of naïve T cells in the presence of anti-CD3/CD28 and IL-2 (for CD8⁺ cell) or Th1/Th2 polarizing conditions (for CD4⁺ cells) showed a low level GFP in primary stimulated cultures. However, upon re-stimulation CD4 and CD8 T cells from Blimp^{gfp/+} and Blimp^{gfp/gfp} mice rapidly became GFP⁺(FIG. 19B). Importantly, Blimp-1 expression in wildtype CD4⁺ effector T cells was confirmed by Western blotting (FIG. 19C). These data demonstrate that the induction of Blimp-1 expression is a component of the maturation of naïe CD4⁺ and CD8⁺ into effector cells.

To further evaluate the Blimp-1 expression after specific antigen stimulation, use was of an infectious disease model. Mice were infected with herpes simplex virus (HSV) and monitored at various time-points post infection for virus specific CD8⁺ T cell primary and memory response using a tetramer specific to the dominant epitope and standard cytotoxic function assays.

These experiments confirm the expression of Blimp-1 in antigen specific T cells and indicate that Blimp^{gfp/gfp} cells are fully functional in their ability to lyse antigen specific target cells.

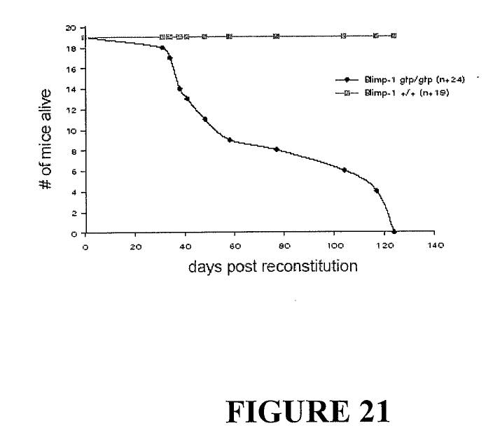

The expanded pool of activated T cells observed in the Blimp^{gfp/gfp} reconstituted mice suggested aberrant responsiveness or deregulated homeostasis in the absence of Blimp-1. In keeping with this conclusion, Blimp^{gfp/gfp} reconstituted mice, displayed pronounced weight loss, ruffled coat and diarrhoea and had to be sacrificed from 6 weeks post-reconstitution. Histological analysis of these mice revealed extensive lymphocyte infiltration and inflammation of a variety of organs including lung, liver and gastrointestinal tract (FIG. 21A). The tissue damage was most pronounced in the lung and intestine, implicating this process in the weight loss, diarrhoea and death observed in these mice. Flow cytometric analysis of lymphoid organs and liver of the reconstituted mice revealed a large expansion of fully activated CD4⁺ and CD8⁺ T cells in all organs (FIG. 2B). The CD4⁺ cells were primarily Th1 biased. Moreover, reconstitution of Rag1^{-/-} mice with Blimp^{gfp/gfp} Rag2^{-/-} foetal liver did not result in any deaths, strongly implicating T cells in the pathology observed (FIG. 21).

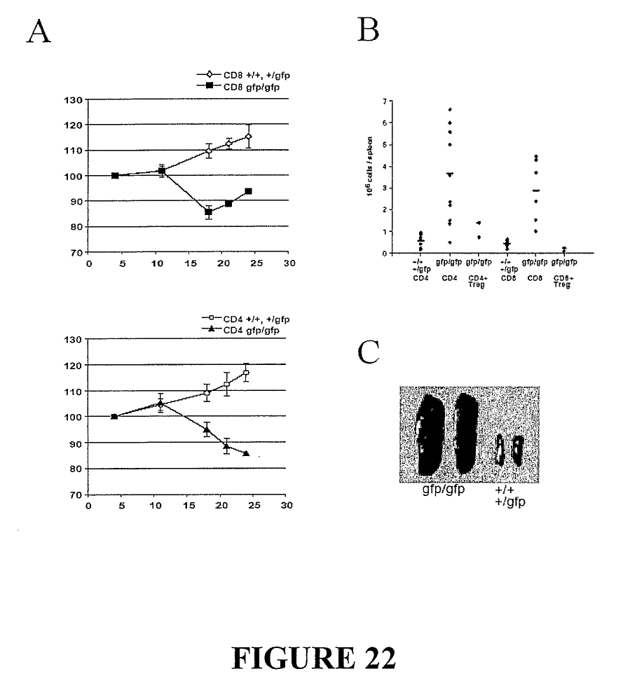

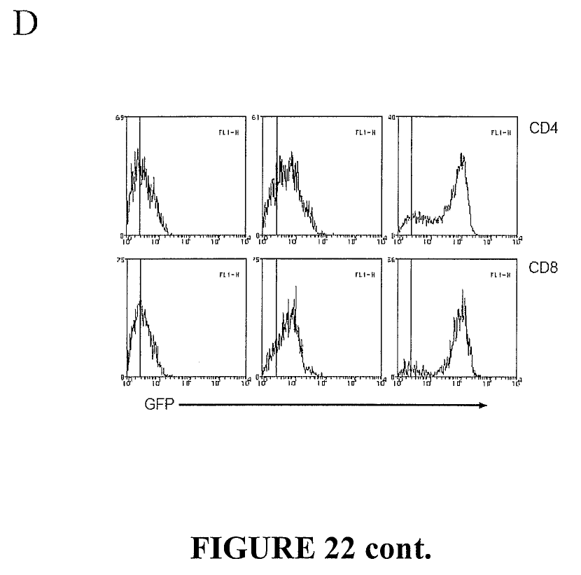

To determine if the uncontrolled T cell expansion is intrinsic to the T cells, adoptive transfer experiments were performed of sorted T cells into Rag1^{-/-}. In these assays wild-type T cells respond to the lymphopenic environment by undergoing limited homeostatic expansion (FIG. 22A). T-cells, 3×10⁶ CD4⁺ or CD8⁺ C57BL/6 or Blimp^{gfp/gfp} were injected into Rag1^{-/-} recipients and the numbers of splenic T cells assessed 3 weeks post-transfer. As shown in FIG. 22A, Blimp^{gfp/gfp} T cells of both lineages had a dramatically expanded homeostatic expansion capacity. Moreover, all the resulting T cells expressed GFP and like the wild-type counterparts displayed an activated phenotype (FIG. 22). The recipients that received Blimp^{gfp/gfp} T cells rapidly developed weight loss, splenomegaly (FIG. 22) and a similar range of lymphocyte infiltration phenotypes as described in FIG. 21. These data demonstrate that Blimp-1 deficient T cells display a dysregulated expansion that results in multi-organ infiltration and death.

Autoimmunity has been associated with the loss of functional CD4⁺CD25⁺ suppressor T cells, Analysis of this population in Blimp^{+/gfp} and Blimp^{gfp/gfp} CD40⁺ T cells revealed moderate to strong expression of GFP in a fraction of CD25⁺ CD4 T cells suggesting a function for Blimp-1 in the regulatory T cell population. Functional analyses in vitro and in vivo confirmed unchanged expression of FoxP3, a key factor for Treg, and other genes associated with Tregs. Blimp deficient cells CD4 cells secrete high levels of IFN and show defective IL-10 secretion.

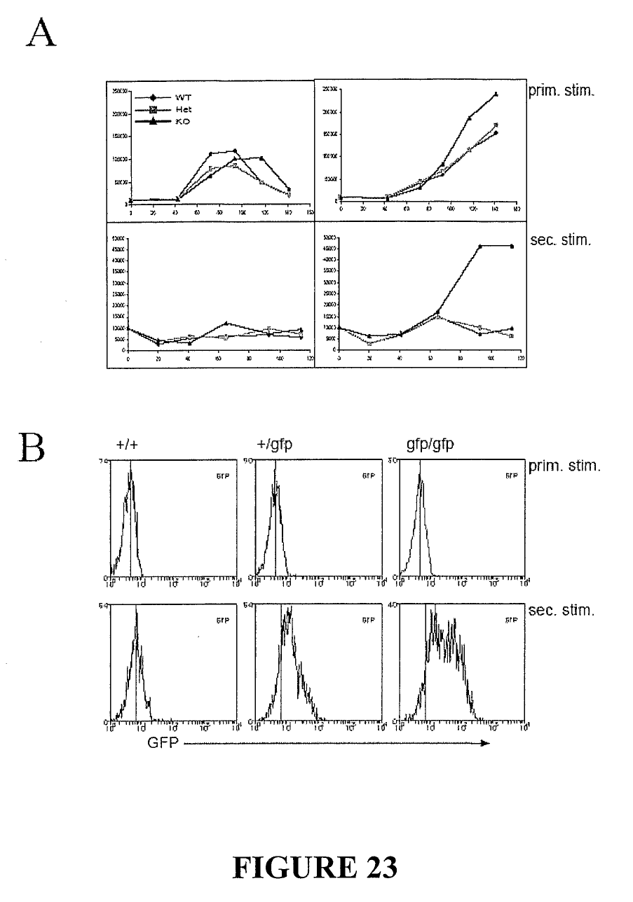

To determine if the dysregulated homeostasis seen in Blimp-1 deficient T cells in vivo also manifests itself as enhanced proliferation in vitro, cultured naïve CD8⁺ T cells were incubated in the presence of anti-CD3/CD28 and cytokines known to regulate cell proliferation and homeostasis (including IL-2, IL-15 and IL-21). Naïve CD8⁺ cells grown for 7 days in the above conditions showed little GFP expression and similar proliferation profiles between the genotypes (FIG. 23A). Similar, results were observed for naïve CD4⁺ cells and those stimulated by PMA/ionomycin combination (data not shown). In contrast, secondary stimulation of the CD8⁺ cells in the presence of IL-2, IL-15 or IL-21 resulted in the strong induction of Blimp-1 expression (FIG. 23A). In agreement with this expression profile, a significant increase in the cumulative cell number was observed in the proliferative response of the Blimp^{gfp/gfp} cells as compared to Blimp-1 sufficient cells. As expected, a similar strong difference in proliferative potential was observed when activated/memory CD44^{high} cells were used for the primary response. This increase was observed in all cytokine conditions but was most pronounced in the presence of IL-21 (FIG. 23B). IL-21 is a T-helper cytokine that was recently shown to be a candidate diabetes susceptibility gene in NOD mice (King, et al. Cell 117:265-277, 2004). In that model IL-21 increased effector T cell turnover resulting in lymphopenia induced homeostatic proliferation and diabetes (King, et al., 2004 (supra)). Interestingly, IL-21 is a potent stimulator of Blimp-1 in B cell terminal differentiation, and was the most efficient inducer of GFP in CD8⁺ T cells suggesting a common role for this cytokine in lymphocyte differentiation.

Molecular and in vitro studies have shown that Blimp-1 is a potent transcriptional repressor that can recruit histone methyl-transferases (Gyory et al., Nat Immunol 5: 299-308, 2004), deacetylases (Yu et al., Mol Cell Biol 20:2592-3603, 2000) and co-repressors of the Groucho family (Ren et al., Genes Dev 13:125-137, 1999) to silence targets. Promoter and microarray studies have identified a number of Blimp-1 targets in the B lineage (Shaffer et al., Immunity 17:51-62, 2002; Shaffer et al., Immunity 21:81-93, 2004). Of these a number including c-myc (Lin et al., Science 276:596, 1997), CIITA (Piskurich et al., Nat Immunol 1:526-532, 2000), are expressed in the T lineage.

### EXAMPLE 11

### Blimp is the Master Regulator of a Conserved Terminal Differentiation Program in all Lymphocytes

In summary, the data described herein demonstrate that Blimp-1 is expressed in activated conventional T cells in a variety of contexts. Blimp expression is essential for normal lymphocyte homeostasis as mice injected with Blimp-1 deficient T cells or reconstituted with mutant stem cells die as a result of an aggressive multi-organ lymphoproliferative disease. Additionally, a cytokines such as IL-21 known to regulate the homeostasis of differentiating T cells, was a strong inducer of Blimp-1 expression and supported enhanced proliferation in vitro in the absence of Blimp-1. Accordingly, Blimp-1 expression may be induced in differentiating effector T cells, not by the initial stimulation but towards the completion of the immune response. Blimp-1 is therefore the first transcription factor identified which functions to regulate the genetic program of T cell contraction and/or memory formation that is essential for immune homeostasis.

B and T lymphocytes share many cellular and genetic similarities during their development such as a common progenitor, the ordered VDJ recombination and similar developmental checkpoints, however whilst the terminal differentiation of B cells to plasma cells is a clear functional end-point (Calame et al., Annu Rev Immunol 8:8, 2003), the final stages of T cell ontogeny are less defined. The similar functions and expression profile of Blimp-1 within the B and T cell lineages raises the intriguing possibility that despite the outwardly dissimilar appearance, Blimp-1 is the master regulator of a conserved terminal differentiation program in all lymphocytes.

Blimp deficiency causes hyperplasia and uncontrolled proliferation while expression of Blimp permits lymphocyte homeostasis and terminal differentiation of haematopoietic cells including ASC, T-cells and B-cells. Accordingly, Cytokines and other immunomodulatory, chemicals, peptides or other small or medium molecular agents which can be screened in the herein described in vitro and in vivo cellular model systems to determine their potential as therapeutic or prophylactic agents.

Accordingly, the present model reporter systems will be useful in assessing the ability of agents to modulate terminal differentiation in cells of the immune system such as T-cells and B-cells

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Arkin et al., Proc. Natl. Acad. Sci. USA 89:7811-7815, 1992.
Altschul et al., Nucl. Acids Res. 25. 3389, 1997.
Angelin-Duclos et al., J Immunol 165:5462, 2000.
Angelin-Duclos et al., Eur J Immunol 32:3765, 2002.
Ausubel (Ed) Current Protocols in Molecular Biology, 5^{th} Edition, John Wiley & Sons, Inc, NY, 2002.
Badovinac et al., Nat Immunol 3:619-626, 2002.
Badovinac et al., Nat Immunol 5:809-817, 2004.
Bonner and Laskey, Eur. J. Biochem. 46: 83, 1974.
Brady et al., J Immunol 172: 2048-2058, 2004.
Calame et al., Annu Rev Immunol 8:8, 2003.
Chambers et al., Immunity 7:885-895, 1997.
Chang et al., Nat Immunol 1:169, 2000.
Chang et al., Mech Dev 117:305, 2002.
Dayhoff et al., Natl. Biomed. Res. Found 5:345-358, 1978.
Delgrave et al., Protein Engineering 6:327-331, 1993.
de Souza et al., Embo J 18:6062, 1999.
Erickson et al,, Science 249: 527-533, 1990.
Fong et al., Proc Natl Acad Sci USA 11:11, 2003.
Fontenot et al., Nat Immunol 4:330-336, 2003.
Gonnet et al., Science 256(5062):1443-1445, 1992.
Gyory et al., Nat Immunol 5: 299-308, 2004.
Hodgson, Bio/Technology 9: 19-21, 1991.
Hogan et al., Manipulating the Mouse Embryo: A Laboratory Manual, Cold Spring Harbour Laboratory Press, CSH NY, 1986.
Hori et al., Science 299:1057-1061, 2003.
Kallies et al., J Exp Med 200:967-977, 2004.
Keller et al., Genes Dev 5:868, 1991.
Khattri et al., Nat Immunol 4:337-342, 2003.
King, et al. Cell 117:265-277, 2004.
Knodel et al., Eur J Immunol 31:1972, 2001.
Kunkel, Proc. Natl. Acad. Sci. USA 82:488-492, 1985.
Kunkel et al., Methods in Enzymol. 154:367-382, 1987.
Kurreck et al, Eur. J. Biochem., 270:1628-1644, 2003.
Lin et al., Science 276:596, 1997.
Mansour et al., Nature 336:348-352, 1988.
Manz et al., Curr Opin Immunol 14:517, 2002.
Marcu et al., Annu Rev Biochem 61:809, 1992.
Marmur et al., J. Mol. Biol. 5: 109, 1962.
Medina et al., Blood 99:2154, 2002.
Messika et al., J Exp Med 188:515, 1998.
Munder et al., Appl., Microbiol. Biotechnol. 52(3): 311-320, 1999.
O'Connor et al., J Exp Med 195:737, 2002.
Ozaki et al., J Immunol 173:5361-5371, 2004.
Pickert, Transgenic Animal Technology: A Laboratory Handbook, Academic Press, San Diego, Calif., 1994.
Piskurich et al., Nat Immunol 1:526-532, 2000.
Remington's Pharmaceutical Sciences, 18^{th} Ed., 1990, Mack Publishing, Company, Easton, Pa., U.S.A.
Ren et al., Genes Dev 13:125-137, 1999.
Robertson et al., Nature 322:445-448, 1986.
Rosenbaum et al., Embo J 9:897, 1990.
Rosenbauer et al., Embo J 21:211-20, 2002.
Sambrook, Molecular Cloning: A Laboratory Manual, 3^{rd} Edition, CSHLP, CSH, NY, 2001.
Schliephake et al., Eur J Immunol 26:268, 1996.
Shaffer et al., Immunity 17:51-62, 2002.
Shaffer et al., Immunity 21:81-93, 2004.
Shapiro, Practical flow cytometry, 3^{rd} Ed., Brisbane, Wiley-Liss, 1995.
Shapiro-Shelef et al., Immunity 19:607, 2003.
Soro et al., J Immunol 163:611, 1999.
Sprent et al., Annu Rev Immunol 20:551-579, 2002.
Sprent et al., Immunol Lett 85:145-149, 2003.
Summerton and Weller, Antisense and Nucleic Acid Drug Development 7: 187-195, 1997.
Tunyaplin et al., Nucleic Acid Research 28(24):4846-4855, 2000.
Turner et al., Cell 77:207, 1994.
Watson et al. “Molecular Biology of the Gene”, Fourth Edition, Benjamin/Cummings, Menlo Park, Calif., 1987.
Ulleras et al., Toxicology 206(2):245-256, 2005.
Underhill et al., Blood 24:24, 2003.
Wells, Methods Enzymol 202: 2699-2705, 1991.
Young et al., Nat. Biotechnol. 16(10): 946-950, 1998.
Youvan et al., Biotechnology et Elia 3: 1-18, 1997.
Yu et al., Mol Cell Biol 20:2592-3603, 2000.

1. A genetically modified cell or non-human organism comprising such cells comprising a modified Blimp (PRDM-1) gene encoding a Blimp polypeptide which when expressed produces Blimp (PRDM-1) or a functional or non-functional part, form, homolog or variant thereof co-expressed with a reporter molecule and wherein the presence of Blimp in the cell is associated with a cellular phenotype and/or a commitment in the cell to terminally differentiate.

2. The cell or organism of claim 1, wherein the Blimp gene encodes a Blimp mRNA transcript comprising a Blimp coding sequence or encoding a functional or non-functional part, form, homolog or variant thereof and a reporter molecule coding sequence.

3. The cell or organism of claim 2, wherein the reporter molecule coding sequence is inserted within an intron of a Blimp allele.

4. The cell or non-human organism of claim 3, wherein the modified Blimp allele is present in homozygous or heterozygous form.

5. The cell or non-human organism of claim 4, wherein the modified Blimp allele is present in heterozygous form.

6. The cell or non-human organism of any one of claims 1 to 5, wherein the modified Blimp allele encodes a functional Blimp transcription factor or a functional part, form, homolog or variant thereof.

7. The cell or non-human organism of any one of claims 1 to 5, wherein the modified Blimp allele encodes a non-functional Blimp transcription factor or a non-functional part, form, homolog or variant thereof.

8. The cell or non-human organism of claim 1, comprising cells or genetic material derived from any organism such as man, non-human primates, livestock, companion or laboratory test organisms, reptilian or amphibian species.

9. The cell or organism of claim 8, derived from a laboratory test animal such as a rodent (including mice), guinea pig, pig, duck, rabbit or sheep.

10. The cell or organism of any one of claims 1 to 5, wherein the cell is a haematopoietic or embryonic cell.

11. The cell or organism of claim 10, wherein the cell is a haematopoietic cell.

12. The cell or organism of claim 11, wherein the cell is a lymphocytic cell.

13. The cell or organism of claim 12, wherein the cell is a cell of the lymphocyte lineage selected from a B-cell and a T-cell.

14. The cell or organism of claim 13, wherein the B-cells are ASC.

15. The cell of claim 14, which is a substantially purified population of ASC.

16. The cell or organism of claim 13, wherein the T-cells are selected from CD40⁺ T-cells and CD8⁺ T-cells.

17. The cell or organism of any one of claims 1 to 16, wherein the detection of the reporter molecule is indicative of a cellular phenotype and/or commitment of a cell to terminally differentiate.

18. The non-human organism of any one of claims 1 to 14, 16 and 17, wherein the organism is provided in the form of embryos, gametes or ES cells.

19. The cell or organism of any one of claims 1 to 18, wherein the reporter molecule is a fluorescent or light emitting reporter molecule.

20. A method for phenotyping and/or monitoring a cell of the haematopoietic system comprising screening a genetically modified haematopoietic cell or non-human animal comprising such cells comprising a modified Blimp gene encoding a Blimp protein which when expressed co-expresses Blimp or functional or non functional part, form, homolog or variant thereof and a reporter molecule, wherein detection of reporter activity is indicative of a cellular phenotype and/or commitment of the cell to terminally differentiate.

21. The method of claim 20, wherein the haematopoietic cell is a cell selected from B-cells, T-cells, dendritic cells, macrophages, natural killer cells, granulocytes, erythrocytes, eosinophils, megakaryocytes, bone marrow, splenic, dermal, or stromal cells and their precursors and derivatives.

22. The method of claim 20 or 21, wherein phenotyping and/or monitoring of cells is achieved by cytometric analysis of a fluorescent or light emitting reporter molecule.

23. The method of claim 20, further comprising isolating or selecting cells which exhibit reporter activity or changes in reporter activity or level from among cells which do not exhibit reporter activity.

24. The method of claim 23, wherein the isolation of reporter-active cells is by flow cytometry, laser scanning cytometry, chromatography and/or other equivalent procedure.

25. The method of claim 23, further comprising selecting reporter-active cells using further selection markers.

26. The method of any one of claims 20 to 25, wherein the cells are ASC identified or isolated in a population of cells of a B-cell lineage.

27. The method of any one of claims 20 to 25, wherein the cells are activated/terminally differentiated T-cells identified or isolated in a mixed population of T-cells.

28. A method for testing the antigenicity or immunogenicity of a vaccine, the method comprising;
(i) administering the vaccine to a genetically modified haematopoietic cell or non-human animal comprising such cells wherein the cell or organism comprises a modified Blimp-1 gene which encodes a Blimp polypeptide which when expressed produces Blimp or a part or fragment or functional form thereof co-expressed with a reporter molecule; and
(ii) testing the cell or organism for the reporter molecule the presence of which is indicative of the ability of the vaccine to induce terminal differentiation in haematopoietic cells.

29. The method of claim 27, wherein the presence of reporter activity is indicative of the ability of the vaccine to promote terminal differentiation in T-cells and/or B-cells.

30. A methods for in vitro or i)? vivo screening for agonists or antagonists of terminal differentiation in haematopoietic cells comprising exposing one or more agent/s to a genetically modified cell or non-human animal comprising such cells wherein the cell or organism comprises a modified Blimp-1 gene which encodes a Blimp polypeptide which when expressed produces Blimp or a part or fragment or functional form thereof co-expressed with a reporter molecule; and testing the cell or organism for the presence or a change in the level of the reporter molecule the presence of which is indicative of the ability of the one or more agent/s to agonise or antagonise terminal differentiation.

31. The method of claim 20, 28 or 30, wherein the cell comprises a modified Blimp gene encoding a Blimp mRNA transcript comprising a Blimp coding sequence or encoding a functional or non-functional part, form, homolog or variant thereof and a reporter molecule coding sequence.

32. The method of claim 31, wherein the reporter molecule coding sequence is inserted within an intron of a Blimp allele.

33. The method of claim 32, wherein the modified Blimp allele is present in homozygous or heterozygous form.

34. The method of claim 33, wherein the modified Blimp allele is present in heterozygous form.

35. The method of claim 31, wherein the modified Blimp allele encodes a functional Blimp transcription factor or a functional part, form, homolog or variant thereof.

36. The method of claim 31, wherein the modified Blimp allele encodes a non-functional Blimp transcription factor or a non-functional part, form, homolog or variant thereof.

37. The method of claim 31 wherein the cells or genetic material are derived from any organism such as man, non-human primates, livestock, companion or laboratory test organisms, reptilian or amphibian species.

38. The method of claim 37, wherein the laboratory test organism is selected from a rodent (including mice), guinea pig, pig, duck, rabbit and sheep.

39. The method of claim 31, wherein the cell is a cancerous or non-cancerous haematopoietic or embryonic cell.

40. The method of claim 39, wherein the cell is a lymphocytic cell.

41. The method of claim 40, wherein the lymphocytic is selected from a B-cell and a T-cell.

42. The method of claim 41, wherein the B-cells are ASC.

43. The method of claim 41, wherein the T-cells are selected from CD40⁺ T-cells and CD8⁺ T-cells.

44. A targeting vector comprising a comprising a modified Blimp (PRDM-1) gene encoding a Blimp polypeptide which when expressed in a target cell produces Blimp (PRDM-1) or a functional or non-functional part, form, homolog or variant thereof co-expressed with a reporter molecule.

45. The targeting vector of claim 44, wherein the Blimp gene encodes a Blimp mRNA transcript comprising a Blimp coding sequence or encoding a functional or non-functional part, form, homolog or variant thereof and a reporter molecule coding sequence.

46. The targeting vector of claim 45, wherein the reporter molecule coding sequence is inserted within an intron of a Blimp allele.

47. The targeting vector of claim 44, wherein the reporter molecule is a GFP.
